(19) United States
(12) Patent Application Publication
(10) Pub. No.: US 2024/0252533 A1
(43) Pub. Date: Aug. 1, 2024

(54) METHODS AND COMPOSITIONS FOR PANCREATIC CANCER EVALUATION AND TREATMENT

(71) Applicant: BOARD OF REGENTS, THE UNIVERSITY OF TEXAS SYSTEM, Austin, TX (US)

(72) Inventors: Raghu KALLURI, Houston, TX (US); Krishnan MAHADEVAN, Houston, TX (US)

(73) Assignee: BOARD OF REGENTS, THE UNIVERSITY OF TEXAS SYSTEM, Austin, TX (US)

(21) Appl. No.: 18/040,586

(22) PCT Filed: Aug. 9, 2021
(86) PCT No.: PCT/US2021/045277

§ 371 Date: Feb. 3, 2023

Related U.S. Application Data
(60) Provisional application No. 63/063,014, filed on Aug. 7, 2020.

(51) Int. Cl. A61K 35/15; A61K 39/00; A61K 45/06; A61P 35/00; C07K 16/28

(52) U.S. Cl. CPC A61K 35/15; A61K 39/4615; A61K 39/4631; A61K 39/464411; A61K 45/06; A61P 35/00; C07K 16/2818; C07K 16/2827; A61K 2239/13; A61K 2239/54

(57) ABSTRACT

Aspects of the present disclosure are directed to methods for treating a subject having pancreatic cancer. Certain aspects relate to treatment with cancer immunotherapy, including immune checkpoint blockade therapy. In some cases, a subject has been determined to have or to have had an inflammatory condition, such as pancreatitis. Further aspects relate to methods for identifying a subject as a candidate for an immune checkpoint blockade therapy.

This application claims benefit of priority of U.S. Provisional Application No. 63/063,014, filed Aug. 7, 2020 which is hereby incorporated by reference in its entirety.

### BACKGROUND

### I. Field of the Invention

Aspects of this invention relate to at least the fields of cancer biology, immunology, and medicine.

### II. Background

While immunotherapy (e.g., checkpoint blockade therapy) aids in control and treatment of certain cancer types, such a benefit in not realized in many others types of cancers. This includes, for example, pancreatic ductal adenocarcinoma (PDAC), breast cancer and others. There is a need in the art for methods and systems for sensitizing such cancers to immunotherapy. Also recognized is a need for methods for stratification and treatment of cancer patients having increased sensitivity to immunotherapy.

### SUMMARY

Aspects of the present disclosure address certain needs by providing methods for selecting and treating subjects with cancer (e.g., pancreatic cancer) having increased sensitivity to immunotherapy and for sensitizing a subject with cancer to immunotherapy treatment. Accordingly, provided herein, in some aspects, are methods for treating a subject with pancreatic cancer comprising providing an immunotherapy to the subject, where the subject has or previously had inflammation of the pancreas. In some embodiments, the disclosed methods comprise providing an immune checkpoint blockade therapy to a subject who has or previously had pancreatitis. Also disclosed are methods for identifying a subject with pancreatic cancer as being sensitive to immunotherapy (e.g., immune checkpoint blockade therapy), the method comprising identifying the subject as having or having had pancreatitis. Further disclosed are methods for treatment of a subject with pancreatic cancer comprising administering dendritic cell vaccine and immune checkpoint blockade therapy.

Embodiments of the disclosure include methods for treating a subject having cancer, methods for diagnosing a subject with cancer, methods for prognosing a subject with cancer, methods for identifying a subject with cancer as sensitive to immunotherapy, methods for sensitizing a subject with cancer to immunotherapy, methods for cancer treatment, methods for identifying subject with cancer as candidates for immunotherapy, and methods for treating a subject having pancreatic cancer. Methods of the disclosure can include 1, 2, 3, 4, 5, 6, or more of the following steps: providing an immunotherapy to a subject, providing an immune checkpoint blockade therapy to a subject, providing an alternative therapy to a subject, determining a subject to have pancreatic cancer, providing a dendritic cell vaccine to a subject, providing two or more types of cancer therapy to a subject, identifying a subject as having pancreatitis, identifying a subject as having had pancreatitis, testing a subject for a symptom of pancreatitis, measuring a level of one or more pancreatic enzymes in a subject, inducing pancreatitis in a subject, and identifying a subject as being a candidate for immunotherapy. Certain embodiments of the disclosure may exclude one or more of the preceding elements and/or steps.

Disclosed herein, in some aspects, is a method for treating a subject with pancreatic cancer, the method comprising providing an immunotherapy to the subject, wherein the subject has been determined to have or to have had pancreatitis. Also disclosed herein, in some aspects, is a method for treating a subject with pancreatic cancer, the method comprising: (a) identifying the subject as having pancreatitis or as having previously had pancreatitis; and (b) providing an immunotherapyto the subject. In some embodiments, (a) comprises testing the subject for one or more symptoms of pancreatitis. In some embodiments, (a) comprises detecting an increased level of one or more pancreatic enzymes in the subject relative to a control or healthy subject. In some embodiments, the one or more pancreatic enzymes comprise amylase or lipase.

Disclosed herein, in some aspects, is a method for treating a subject with pancreatic cancer, the method comprising determining whether the subject has or has previously had pancreatitis and (a) providing an immunotherapyto the subject if the subject is determined to have or to have previously had pancreatitis; or (b) providing an alternative cancer therapy to the subject if the subject is determined to have never had pancreatitis, wherein the alternative cancer therapy does not comprise an immunotherapy. In some embodiments, the alternative cancer therapy is chemotherapy, hormone therapy, radiation therapy, or surgery. In some embodiments, determining whether the subject has or has previously had pancreatitis comprises testing the subject for one or more symptoms of pancreatitis. In some embodiments, determining whether the subject has or has previously had pancreatitis comprises detecting an increased level of one or more pancreatic enzymes in the subject relative to a control or healthy subject. In some embodiments, the one or more pancreatic enzymes comprise amylase or lipase.

Disclosed herein, in some aspects, is a method for treating a subject with pancreatic cancer, the method comprising: (a) inducing pancreatitis in the subject; and (b) subsequent to (a), providing to the subject an immunotherapy. In some embodiments, (a) comprises providing an infectious agent to the subject. In some embodiments, (a) comprises pancreatic surgery.

In some embodiments, the pancreatitis is chronic pancreatitis. In some embodiments, the pancreatitis is acute pancreatitis. In some embodiments, the pancreatic cancer is pancreatic ductal adenocarcinoma (PDAC). The immunotherapy may be any immunotherapy including, for example, an immune checkpoint blockade therapy, CAR-T cell therapy, adoptive T cell therapy, dendritic cell vaccine, etc. In some embodiments, the immunotherapy is an immune checkpoint blockade therapy. In some embodiments, the immune checkpoint blockade therapy comprises providing to the subject an antibody or antibody-like molecule capable of binding to an immune checkpoint protein. In some embodiments, the immune checkpoint blockade therapy comprises providing to the subject a cell comprising a chimeric antigen receptor (CAR) capable of binding to an immune checkpoint protein. In some embodiments, the immune checkpoint protein is CTLA-4, PD-1, PDL1, PD-2, IDO, LAG3, or TIM-3. In some embodiments, the immune checkpoint protein is PD-1. In some embodiments, the immune checkpoint blockade therapy comprises at least, at most, or exactly 1, 2, 3, 4, or 5 immune checkpoint inhibitors. In some embodiments, the immune checkpoint blockade therapy comprises at least two immune checkpoint inhibitors. In some embodiments, the two or more immune checkpoint inhibitors comprise two or more of an anti-PD-1 antibody, an anti PDL1 antibody, and an anti-CTLA4 antibody.

In some embodiments, pancreatic cancer tissue from the subject was determined to comprise CD11c+ dendritic cells. In some embodiments, pancreatic tissue from the subject was determined to comprise tertiary lymphoid structures. In some embodiments, the method further comprises providing to the subject a dendritic cell vaccine.

Further disclosed herein, in some aspects, is a method for treating a subject with pancreatic cancer, the method comprising administering an effective amount of a dendritic cell vaccine and an immunotherapy (e.g., immune checkpoint blockade therapy) to the subject. In some embodiments, the dendritic cell vaccine and the immunotherapy are administered substantially simultaneously. In some embodiments, the dendritic cell vaccine and the immunotherapy are administered sequentially. In some embodiments, the dendritic cell vaccine is administered prior to the immunotherapy. In some embodiments, the immunotherapy is administered prior to the dendritic cell vaccine.

In some embodiments, the dendritic cell vaccine is an autologous dendritic cell vaccine. In some embodiments, the dendritic cell vaccine comprises conventional dendritic cells (cDCs). In some embodiments, the cDCs are conventional type 1 dendritic cells (cDC1s). In some embodiments, the subject was previously treated for pancreatic cancer. In some embodiments, the subject was previously treated with an immunotherapy. In some embodiments, the subject was determined to be resistant to the previous treatment. In some embodiments, the method further comprises providing to the subject an additional cancer therapy. In some embodiments, the additional cancer therapy is chemotherapy, radiation therapy, hormone therapy, surgery, or immunotherapy.

Disclosed herein, in some aspects, is a method for identifying subjects with pancreatic cancer as candidates for immune checkpoint blockade therapy, the method comprising: (a) determining whether each subject of a group of subjects with pancreatic cancer has or has previously had pancreatitis; and (b) identifying subjects from the group of subjects that have or have previously had pancreatitis as candidates for immunotherapy.

Also disclosed, in certain aspects, is a method for treating a subject for pancreatic cancer, the method comprising administering an immunotherapy to a subject determined to have tertiary lymphoid structures in pancreatic cancer tissue from the subject. In some embodiments, the immunotherapy is an immune checkpoint blockade therapy.

Further disclosed, in some aspects, is a method for treating a subject for pancreatic cancer, the method comprising: (a) detecting tertiary lymphoid structures in pancreatic tissue of the subject; and (b) administering an immunotherapy to the subject.

Also disclosed, in some embodiments, is a method for treating a subject for pancreatic cancer, the method comprising: (a) inducing formation of tertiary lymphoid structures in pancreatic tissue of the subject; and (b) administering an immune checkpoint blockade therapy to the subject.

Throughout this application, the term “about” is used to indicate that a value includes the inherent variation of error for the measurement or quantitation method.

The use of the word “a” or “an” when used in conjunction with the term “comprising” may mean “one.” but it is also consistent with the meaning of “one or more.” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise.” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

“Individual, “subject,” and “patient” are used interchangeably and can refer to a human or non-human.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1E: Pancreatitis increases tertiary lymphoid structures and accelerates tumorigenesis in KC mice. (FIG. 1A) Schematic representation of experimental time line for induction of acute and Chronic pancreatitis. (FIG. 1B) Representative H & E stained section of entire pancreatic tissue with the TLS in KC mice with AP and CP. (FIG. 1C) Quantification of number of TLS in the pancreas of KC mice with AP and CP. (FIGS. 1D-1E) Representative H & E and CK 19 immunostaining with quantification of pancreatic tissue in KC mice with and without AP. Scale bars indicate 100 um. In C and E, data represents mean±SD, two-way analysis of variance (for tumor histology) and unpaired T test (parametric or non-parametric) was used for statistical analysis. P values: ns—not significant. *<0.05, **<0.01, ***<0.001, ****<0.0001.

FIGS. 2A-2F: Analysis of myeloid and dendritic cell infiltration in KC mice with acute pancreatitis. (FIGS. 2A-2B) Representative CD11c immunostaining with quantification of PanIN lesions and TLS in KC mice with and without AP. (FIGS. 2C-2D) Representative CD11b immunostaining with quantification of PanIN lesions and TLS in KC mice with and without AP. (FIGS. 2E-2F) Representative CD11c—MHC-II immunostaining with quantification of PanIN lesions in KC mice with and without AP. Scale bars indicate 100 um. In B, D and F, data represents mean±SD, unpaired T test (parametric or non-parametric) was used for statistical analysis. P values: ns—not significant. *<0.05, **<0.01, ***<0.001, ****<0.0001.

FIGS. 3A-3B: Analysis of T cell infiltration in KC mice with and without AP. (FIG. 3A) Representative CD4 and CD8 immunostaining with quantification of PanIN lesions and TLS in KC mice with and without AP. (FIG. 3B) Representative CD4-Foxp3. CD4-GATA3. CD4-RORgt and CD-T-bet co-staining with quantification of PanIN lesions and TLS (only CD4-Foxp3) in KC mice with and without AP. Scale bars indicate 100 um. In A and B, data represents mean±SD, unpaired T test (parametric or non-parametric) was used for statistical analysis. P values: ns—not significant.

FIGS. 4A-4D: Immunolabeling of tumors and lymphoid tissue of CD4^{-/-} and CD8^{-/-} mice for T cells. (FIGS. 4A-4B) CD4 and CD8 immunostaining (FIG. 4A) and corresponding quantification (FIG. 4B) of % of CD4+ or CD8+ area of thymus and spleen of KPC, KPC CD4^{-/-} and KPC CD8/mice. n=2-4 mice in each group. (FIG. 4C) CD4 and CD8 immunostaining and corresponding quantification (FIG. 4D) of KPC, KPC CD4^{-/-} and KPC CD8/mice tumors and tertiary lymphoid structures (TLS). n=3-7 mice in each group. Data are presented as the mean±SD. Significance was determined by Unpaired T-Test (parametric or non-parametric). *P<0.05, ****P<0.0001, ns: not significant.

FIGS. 5A-5H: CD4⁺ or CD8⁺ T cells do not alter primary tumor growth, however CD4⁺ T cells promote metastasis in KPC mice. (FIG. 5A) Kaplan-Meier survival curve of baseline KPC (n=19), KPC CD4^{-/-} (n=13) and KPC CD8^{-/-} (n=11) mice. (FIGS. 5B-5D) Representative H&E images and Ki67 immunostaining with quantification of endpoint KPC, KPC CD4^{-/-} and KPC CD8^{-/-} tumors (n=4-8 mice in each group). (FIG. 5E) End point tumor weights of KPC (n=19), KPC CD4^{-/-} (n=11) and KPC CD8^{-/-} (n=11) mice. (FIG. 5F) Representative H&E images and CK 19 immunostaining of liver metastasis of KPC and KPC CD8−/− mice. (FIGS. 5G-5H) Quantification of liver metastasis (FIG. 5G), and lung metastasis (FIG. 5H), of KPC, KPC CD4^{-/-} and KPC CD8^{-/-} mice. In (FIGS. 5C, 5D, 5E, 5G, 5H), data represents mean±SD. Significance was determined by log-rank test (FIG. 5A), two-way ANOVA (FIG. 5B) and unpaired (parametric or non-parametric) T test (FIGS. 5D, 5E, 5G, 5H). *P<0.05, ns—not significant. Scale bars, 100 um.

FIGS. 6A-6E: T cells have no impact on tumorigenesis, whereas CD4⁺ T cells promote tumorigenesis in KC mice with pancreatitis. (FIGS. 6A-6B) Representative H&E and CK19 immunostaining images with quantification of PanIN lesions of (age matched sacrifice at 6m from birth); KC (n=5). KC CD4^{-/-} (n=6) and KC CD8^{-/-} (n=6) mice. (FIG. 6C) Schematic representation of AP and CP induction with experimental treatment time points. (FIGS. 6D-6E) Representative H&E and CK19 immunostaining of PanIN lesions of mice with AP and CP. AP—KC (n=5). AP—KC CD4^{-/-} (n=5) and AP—KC CD8^{-/-} (n=6). In (FIGS. 6B, 6E) data are presented as mean±SD. Significance was determined by unpaired T test (parametric or non-paramteric) and two way analysis of variance (ANOVA). **P<0.01, ***P<0.001, ****P<0.0001, ns: not significant. Scale bars indicate 100 μm.

FIGS. 7A-7I: CD4⁺ T cells promote tumorigenesis in KC mice with pancreatitis by restraining dendritic cells. (FIG. 7A) Schematic representation of AP induction with αCD11c or isotype antibody treatment time points in KC CD4^{-/-} mice. (FIGS. 7B-7C) Representative H&E and CK19 immunostaining with quantification of PanIN lesions of with αCD11c or isotype treated KC CD4^{-/-} mice. AP—KC CD4^{-/-} (n=5). AP—KC CD4^{-/-} Isotype (n=4) and AP—KC CD4^{-/-} αCD11c (n=4) mice. (FIGS. 7D-7I) Representative images of CD4. CD8 and CD11c co-staining with quantification of indicated groups and cell types in KC and KC CD4^{-/-} mice with and without CP (FIG. 7D). Quantification of CD11c⁺ cells in KC and KC—CP mice (FIG. 7E), CD8⁺ T cells in KC and KC CD4^{-/-} mice (FIG. 7F), number of CD11c⁺ and CD4⁺ cells in contact in KC and KC—CP mice (FIG. 7G), number of CD11c⁺ and CD8⁺ cells in contact in KC and KC—CP mice (FIG. 7H), CD8⁺ T cells in KC CD4^{-/-} and AP—KC CD4^{-/-} mice (FIG. 7H). In (FIGS. 7C, 7E, 7F, 7G, 7H, 7I), data represents mean±SD. Significance was determined by two-way ANOVA (FIG. 7C) and Unpaired (parametric or non-parametric) T-test (FIGS. 7C, 7E, 7F, 7G, 7H, 7I). *P<0.05, **P<0.01, ****P<0.0001, ns— not significant. Scale bars indicate 100 μm.

FIGS. 8A-8C: (FIG. 8A) Individual channel images from FIG. 7D. (FIG. 8B) Quantification of CD11c+ DCs in the TLS of KC and KC—CP mice

FIGS. 9A-9H: Characterization of pancreatic immune infiltrates of wildtype mice with pancreatitis. (FIG. 9A) Schematic representation of acute and chronic pancreatitis induction (caerulein injection 4 times a day indicated by arrow heads) with experimental time points for flowcytometry and CyTOF. (FIG. 9B) Representative viSNE plots on CD45⁺ cells of WT (n=16 mice, pancreas from 4 mice combined per sample) and WT-AP mice pancreas (n=8, pancreas from 2 mice combined per sample). (FIG. 9C) Heat map of WT pancreas infiltrating immune cell metaclusters displaying expression values of individual parameters normalized to the maximum mean value across metaclusters. (FIGS. 9D-9G) Relative frequencies of metaclusters for indicated cell types. B cell metaclusters (FIG. 9D). T metaclusters (FIG. 9E). DC metaclusters (FIG. 9F), and myeloid Metaclusters (FIG. 9G). (FIG. 9H) Immune cell populations from pancreas of WT (n=20 mice, pancreas from 4 mice combined per sample) and WT-CP mice (n=10, pancreas from 2 mice combined per sample) by flowcytometry. Analysis in (FIGS. 9B-9G), were performed on CyTOF data. In (D-G) data are presented as box and whisker plots. In (FIG. 9H) data are presented as mean±SD. Significance was determined by Unpaired T test (parametric or non-parametric) (FIGS. 9D, 9E, 9F, 9G, and 9H). *P<0.05, **P<0.01, ***P<0.001, ****P<0.0001, ns: not significant

FIGS. 10A-10F: (FIG. 10A) Serum amylase and lipase levels in WT (n=5) and WT-CP (n=10) mice 1 w following start of caerulein injections. (FIGS. 10B-10C) Immune cell populations identified by manual gating of CyTOF data in WT. WT-AP (FIG. 10B) and iKPC*, iKPC—CP (FIG. 10C) mice. CD3⁺, CD3⁺CD4⁺, CD3⁺CD8⁺ T cells. CD11c⁺DCs, CD11b⁺ myeloid cells and CD19⁺ B cells. (FIGS. 10D-10F) Immune cell populations from pancreas of iKPC (n=3 mice) and iKPC*—CP mice (n=4 mice) by flowcytometry. cDC2s (CD11c⁺ B220⁻ CD172a⁺ CD64⁻Ly6c⁻ CD11b⁺ cells) (FIG. 10D), pDCs (CD11c⁺ B220⁺ SIGLEC H⁺ cells) (FIG. 10E), mDCs (CD11c⁺ B220⁻ CD172a⁺ CD64⁻ Ly6c⁺ cells) were measured as a percentage of CD45⁺Lin (CD19, Ly6G, CD3, NK1.1)⁻ cells. In (FIGS. 10A-10F), data are presented as mean±SD. Significance was determined by Unpaired T test (parametric or non-parametric) (FIGS. 10A-10F). *P<0.05, **P<0.01, ***P<0.001, **P<0.0001, ns: not significant

FIGS. 11A-11J: Characterization of pancreatic immune infiltrates of iKPC* mice with pancreatitis. (FIG. 11A) Schematic representation of chronic pancreatitis induction (caerulein injection 4 times a day indicated by arrow heads) with experimental time points for flowcytometry and CyTOF. (FIG. 11B) Representative viSNE plots on CD45+ cells of iKPC* (n=4 mice) and iKPC*—CP mice pancreas (n=6 mice). (FIG. 11C) Heat map of iKPC* pancreas infiltrating immune cell metaclusters displaying expression values of individual parameters normalized to the maximum mean value across metaclusters. (FIGS. 11D-11F) Relative frequencies of metaclusters for indicated cell types. B and T cell metaclusters (FIG. 11D). DC metaclusters (FIG. 11E), and myeloid Metaclusters (FIG. 11F). (FIG. 11G) Immune cell populations from pancreas of iKPC (n=3 mice) and iKPC*—CP mice (n=4 mice) by flowcytometry. (FIG. 11H) CD11c⁺CD86⁺ DCs and CD11c⁺CD40⁺ DCs were measured as a percentage of CD45⁺ cells. CD11c⁺MHC-II⁺F4/80+ DCs (FIG. 11I), cDC1s (CD11c⁺B220⁻CD172a⁻CD64⁻Ly6c⁻CD11b⁻MHC-II⁺ XCR1⁺ cells) (FIG. 11J) were measured as a percentage of CD45⁺Lin (CD19, Ly6G, CD3, NK1.1)⁻ cells. Analysis in (FIGS. 11B-11F) were performed on CyTOF data. In (FIGS. 11D-11F) data are presented as box and whisker plots. In (FIGS. 11G-11J) data are presented as mean±SD. Significance was determined by Unpaired T test (parametric or non-parametric) (FIGS. 11D, 11E, 11F, 11G, 11H, 11I and 11J). *P<0.05, **P<0.01, ***P<0.001, ****P<0.0001, ns: not significant.

FIGS. 12A-12F: Analysis of T cell populations in WT, WT-AP and WT, CP mice. (FIGS. 12A-12G) Immunophenotyping analysis of flowcytometry data of indicated groups. CD8⁺Ki67⁺(FIG. 12A). CD8⁺GrnzB⁺(FIG. 12B), CD8⁺T-bet⁺(FIG. 12C), CD4⁺ T-bet⁺(FIG. 12D), CD8⁺PD1⁺(FIG. 12E), CD8⁺TIM3⁺(FIG. 12F) and CD4⁺Foxp3⁺ cells(FIG. 12G) measured as a percentage of CD45⁺ cells. In (FIGS. 12A-12G) data are presented as mean±SD. Significance was determined by Unpaired T test (parametric or non-parametric) (FIGS. 12A-12G). **P<0.01, ***P<0.001, ****P<0.0001, ns: not significant.

FIGS. 13A-13H: Pancreatitis recruits activated dendritic cells to the pancreas of KPC mice. (FIG. 13A) Schematic representation of pancreatitis induction and experimental time points in KPC mice at 10w (n=5) and KPC—CP mice at 10w (n=6). (FIG. 13B) Pancreas weights of KPC—10w and KPC—CP mice. (FIG. 13C) Representative viSNE plots on CD45+ cells of KPC—10w and KPC—CP mice pancreas. (FIG. 13D) Heat map of WT pancreas infiltrating immune cell metaclusters displaying expression values of individual parameters normalized to the maximum mean value across metaclusters. (FIGS. 13E-13G) Relative frequencies of metaclusters for indicated cell types. B and T cell metaclusters (FIG. 13H), DC metaclusters (FIG. 13I), and myeloid Metaclusters (FIG. 13J). Analysis in (FIGS. 13E-13H), were performed on CyTOF data. In (FIGS. 13E-13G) data are presented as box and whisker plots. In (FIGS. 13B, 13H) data are presented as mean±SD. Significance was determined by Unpaired T test (parametric or non-parametric) (FIGS. 13B, 13E, 13F, 13G, and 13H). *P<0.05, **P<0.01, ***P<0.001, ****P<0.0001, ns: not significant. Scale bars indicate 100 μm.

FIGS. 14A-14L: DC vaccine and CP sensitizes the iKPC* orthotopic mice to checkpoint immunotherapy. (FIG. 14A) Schematic representation of orthotopic injection of iKras cell line in B6 mice. CP induction and DC vaccine with αCTLA4/PD1 or isotype treatment time points. (FIG. 14B) Kaplan-Meier survival curve of iKras mice treated with isotype. αCTLA4+αPD1. CP: isotype. CP: αCTLA4+αPD1. DC vaccine+αCTLA4+αPD1 antibody, n=5-6 mice in each group. (FIGS. 14C-14G) Immunophenotyping data on flowcytometry data of indicated groups. (FIG. 14C) CD3⁺, CD3⁺CD4⁺, CD3⁺CD8⁺ T cells, CD8⁺Ki67⁺ and CD8⁺GrnzB⁺ cells were measured as a percentage of CD45⁺ cells. (FIG. 14D) CD4⁺T-bet⁺(Th1) and CD8⁺T-bet⁺ cells and (FIG. 14E) CD4⁺Foxp3⁺ cells were measured as a percentage of CD45⁺ cells. CD8/Treg ratio (FIG. 14F) and Th1/Treg ratio (FIG. 14G). (FIG. 14H) CD3⁺, CD3⁺CD4⁺, CD3⁺CD8⁺ T cells. CD8⁺Ki67⁺ and CD8⁺GrnzB⁺ cells of indicated groups were measured as a percentage of CD45⁺ cells. (FIG. 14I) CD8⁺T-bet⁺ cells were measured as a percentage of CD45⁺ cells. (FIG. 14I) CD4⁺Foxp3⁺ cells and (FIG. 14J) CD4⁺T-bet⁺(Th1) and CD8⁺T-bet⁺ cells were measured as a percentage of CD45⁺ cells. In (FIGS. 14C-14L), data represents mean±SD. Significance was determined by log rank test (FIG. 14B) and unpaired T test (parametric or non-parametric) (FIGS. 14C-14L). *P<0.05, P<0.01, ***P<0.001, ****P<0.0001, ns—not significant.

FIGS. 15A-15F: (FIGS. 15A-15F) Individual Kaplan Meier survival curves of indicated groups of orthotopic iKPC* tumor mice from FIG. 14B.

FIGS. 16A-16J: (FIG. 16A) Tumor weights of isotype and DC vaccine+αCTLA4+αPD1 treated mice sacrificed on day 21 following orthotopic iKras injection. (FIG. 16B) Immunophenotyping analysis for DC subsets (cDC1s (CD11c⁺ B220⁻ CD172a⁻ CD64⁻ Ly6c⁻ CD11b⁻ MHC-II⁺ XCR1⁺ cells), cDC2s (CD11c⁺ B220⁻CD172a⁺CD64⁻Ly6c⁻CD11b⁺ cells), mDCs (CD11c⁺B220⁻CD172a⁺ CD64⁻Ly6c⁺ cells) and pDCs (CD11c⁺B220⁺ SIGLEC H⁺ cells)) measured as a percentage of CD45⁺Lin (CD19, Ly6G, CD3, NK1.1)⁻ cells in iKPC* isotype and DC vaccine+αCTLA4+αPD1 treated mice. (FIGS. 16C, 16D) Immunophenotyping analysis of exhaustion markers on T cells of indicated groups. CD4⁺ PD1⁺, CD8⁺ PD1⁺ cells (C) and CD4⁺ TIM3⁺, CD8⁺ TIM3⁺ cells(D) measured as a percentage of CD45⁺ cells. (FIG. 16E) Immunophenotyping analysis of activation and memory marker CD69 on T cells as a percentage of CD45⁺ cells. (FIG. 16F) Tumor weights of isotype, CP. αCTLA4+αPD1 and CP: αCTLA4+αPD1 treated mice sacrificed on day 14 following orthotopic iKras injection. (FIGS. 16H-16I) Immunophenotyping analysis of exhaustion or activation markers on T cells of indicated groups. CD8⁺ PD1⁺ and CD4⁺ PD1⁺ cells (H) and CD4⁺ TIM3⁺, CD8⁺ TIM3⁺ cells (FIG. 16I) measured as a percentage of CD45⁺ cells. (FIG. 16J) Immunophenotyping analysis of activation and memory marker CD69 on T cells as a percentage of CD45⁺ cells. In (FIGS. 16A-16J), data represents mean±SD and significance was determined by unpaired T-test (parametric or non-parametric). *P<0.05, **P<0.01, P<0.001, ns—not significant.

FIGS. 17A-17F: Pancreatitis renders the KC and KPC689 orthotopic mice sensitive to checkpoint blockade. (FIG. 17A) Schematic representation of AP induction and αCTLA4/PD1 or isotype treatment time points in KC mice. (FIGS. 17B-17C) Representative H&E and CK19 immunostaining with quantification of PanIN lesions of with CTLA4/PD1, αPD1 or isotype treated mice; AP—KC (n=5), AP—KC Isotype (n=5), AP—KC αPD1 (n=4) and AP—KC αCTLA4+αPD1 (n=4). (FIG. 17D) Schematic representation of orthotopic injection of KPC 689 cells in B6 mice, CP induction and DC vaccine with αCTLA4/PD1 or isotype treatment time points. (FIG. 17E) Quantification of IVIS imaging of baseline on d7, follow up on d18, imaging on d85 before re-challenge and on d95 in indicated experiments groups. (FIG. 17F) Kaplan-Meier survival curve of indicated groups, n=6-9 mice in each group. In (FIGS. 17C, 17E), data represents mean±SD. Significance was determined by two-way ANOVA and Unpaired (parametric or non-paramteric) T tests (FIGS. 17C, 17E), and log rank test (FIG. 17F). *P<0.05, **P<0.01, ***P<0.001, ns—not significant. Scale bars indicate 100 μm.

FIGS. 18A-18H: (FIGs. A-H) Individual Kaplan Meier survival curves of indicated groups of orthotopic KPC689 tumor mice. Significance was determined by log rank test. *P<0.05, ****P<0.0001, ns—not significant.

FIGS. 19A-19D: (FIG. 19A) IVIS imaging of baseline on d7, follow up on d18, imaging on d85 before re-challenge and on d95 in indicated experiments groups. (FIGS. 19B-19E) KPC689 GFP-Luc containing and parental tumor cell lysates demonstrated similar levels of CD8⁺ T cell activation. Bone marrow-derived CD103⁺ cDC1s stimulated with Poly I:C and/or tumor cell lysates of KPC GFP-expressing cells or parental KPC tumors. Cells were co-cultured with CFSE-labeled wild type CD8⁺ T cells with DCs. OT-I splenic CD8⁺ T cells were cocultured with CD103⁺ cDC1s stimulated with ovalbumin and/or Poly I:C as a positive control. (FIG. 19B) % CFSER CD8⁺ Tcells. (FIG. 19C) % CD25⁺ CD8⁺Tcells, (FIG. 19D) CD25 and (FIG. 19E) PD1 (MFI) on T cells was quantified via flow cytometry. (n=5 in each group). Immunophenotyping analysis of CD3⁺ (T cells), CD3⁺ CD4⁺ T cells, CD3⁺ CD8⁺ T cells, CD11c⁺ DCs, CD11b⁺ myeloid cells (E), CD4⁺ Foxp3⁺ (T regs), CD8⁺GrnzB⁺, CD11c⁺CD40⁺, CD11c⁺CD86⁺ cells measured as a percentage of CD45⁺ cells in orthotopic KPC 689 tumor mice 45 days after orthotopic injection. In (FIGS. 19B-19E), data represents mean±SD. Significance was determined by unpaired T test (parametric or non-parametric). *P<0.05, **P<0.01, ns—not significant.

FIGS. 20A-20B: (FIGS. 20A-20B) Tumor histology (H & E) analysis and quantification in mice at end-point or at sacrifice. In (FIG. 20B), data represents mean±SD. Significance was determined by two-way ANOVA. ****P<0.0001, ns—not significant.

FIGS. 21A-12F: Tumor infiltrating dendritic cells correlate with CD8⁺T cell infiltration and better prognosis in human PDAC. (FIG. 21A) Representative immunostaining for CD4, CD8, Foxp3 and CD11c⁺ cells in human PDAC TMA samples. (FIG. 21B) Linear regression analysis of CD11c⁺ DCs with each T cell group viz. CD4⁺ T. CD8⁺ T and CD4+ Foxp3⁺ T cells in each TMA; (n=119) PDAC patients. (FIGS. 21C-21D) Kaplan Meier survival curves for DSS of CD11c high (n=56) Vs. CD11c low (n=63) tumors (FIG. 21C), CD8 high (n=59) Vs. CD8 low (n=61) tumors (FIG. 21D), CD4 high (n=58) Vs. CD4 low (n=62) tumors (FIG. 21E) and Treg high (n=55) Vs. Treg low (n=65) tumors (FIG. 21F). Significance was determined by log rank test (FIGS. 21C, 21D, 21E and 21F). Scale bars indicate 100 μm. ***P<0.001, ns—not significant.

FIG. 22: PDAC patients were stratified as ‘high’ and ‘low’ for each cell type based on median value among all patients in the TMA cohort.

FIGS. 23A-23B: Analysis of TCGA dataset. (FIGS. 23A-23B) Kaplan meier survival curves for DSS of BATF3 low and high expression based on median expression BATF3 high (n=65), BATF3 low (n=44) (FIG. 23A), and CD11c: ITGAX high (n=54) and low (n=55) (FIG. 23B) expression, ns—not significant.

### DETAILED DESCRIPTION

The present disclosure is based at least in part, on the surprising discovery that pancreatic cancer preceded by or associated with inflammation independent of cancer is responsive to cancer immunotherapy, including immune checkpoint blockade therapy. As disclosed herein, acute and/or chronic pancreatitis, when associated with pancreatic cancer, relieves immunosuppression and enables efficacy of immune checkpoint blockade therapy. Accordingly, in some embodiments, disclosed are methods for treating pancreatic cancer comprising providing an immune checkpoint blockade therapy to a subject having or suspected or having cancer, wherein the subject has or has previously had pancreatic inflammation (e.g., organ damage, pancreatic fibrosis, and/or pancreatitis). Further aspects disclose methods for stratifying pancreatic cancer patients based on a history of inflammation, including pancreatitis. For example, embodiments are directed to methods for identifying a subject as being a candidate for immune checkpoint blockade therapy by identifying the patient as having or having previously had pancreatic inflammation such as pancreatitis. Also disclosed are methods for treatment of pancreatic cancer comprising administration of a dendritic cell vaccine and immune checkpoint blockade therapy.

### I. Therapeutic Methods

Aspects of the disclosure are directed to compositions and methods for therapeutic use. The compositions of the disclosure may be used for in vivo, in vitro, or ex vivo administration. The route of administration of the composition may be, for example, intracutaneous, subcutaneous, intravenous, local, topical, and intraperitoneal administrations.

### A. Cancer Therapy

In some embodiments, the disclosed methods comprise administering a cancer therapy to a subject or patient. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be an immune checkpoint therapy. Any of these cancer therapies may also be excluded. Combinations of these therapies may also be administered.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. Any of the disclosed methods or compositions may be employed for treatment of any of any cancer type. For example, aspects of the present disclosure include treatment of any cancer with (a) a dendritic cell vaccine, and (b) an additional immunotherapy such as an immune checkpoint blockade therapy. Additional aspects include induction of inflammation at a tumor tissue, followed by treatment with immunotherapy (e.g., immune checkpoint blockade therapy). Further aspects include induction of tertiary lymphoid structure formation at a tumor tissue, followed by treatment with immunotherapy (e.g., immune checkpoint blockade therapy).

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's discase, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pincaloma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; cosinophilic leukemia; monocytic leukemia; mast cell leukemia; megakaryoblastic leukemia; myeloid sarcoma; and hairy cell leukemia.

In some embodiments, disclosed are methods for treating cancer originating from the pancreas. In some embodiments, the cancer is pancreatic cancer. In some embodiments, the cancer is pancreatic ductal adenocarcinoma (PDAC).

In some embodiments, disclosed are methods for treating cancer originating from the breast. In some embodiments, the cancer is breast cancer.

### B. Cancer Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Various immunotherapies are known in the art, and examples are described below.

### 1. Checkpoint Inhibitors and Combination Treatment

Embodiments of the disclosure may include administration of immune checkpoint inhibitors, examples of which are further described below. As disclosed herein, “immune checkpoint blockade therapy” (also “immune checkpoint therapy”, “checkpoint blockade immunotherapy,” or “CBI”), refers to cancer therapy comprising providing one or more immune checkpoint inhibitors to a subject having or suspected of having cancer. In some aspects, an immune checkpoint blockade therapy of the disclosure comprises at least, at most, or exactly 1, 2, 3, 4, or 5 immune checkpoint inhibitors, or more. In some aspects, an immune checkpoint blockade therapy comprises two or more immune checkpoint inhibitors (e.g., PD-1 inhibitor and CTLA4 inhibitor).

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO 01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or VH and/or VL domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the claimed disclosure can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1, CDR2, and CDR3 domains of the VH region of an anti-LAG3 antibody, and the CDR1, CDR2 and CDR3 domains of the VL region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+ Th1 and CD8+ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or VH and/or VL domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed disclosure can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

In some embodiments, the inhibitor comprises the heavy and light chain CDRs or VRs of an anti-TIM-3 antibody. Accordingly, in one embodiment, the inhibitor comprises the CDR1, CDR2, and CDR3 domains of the VH region of an anti-TIM-3 antibody, and the CDR1, CDR2 and CDR3 domains of the VL region of an anti-TIM-3 antibody. In another embodiment, the antibody has at least about 70, 75, 80, 85, 90, 95, 97, or 99% (or any derivable range therein) variable region amino acid sequence identity with the above-mentioned antibodies.

### C. Activation of Co-Stimulatory Molecules

In some embodiments, the immunotherapy comprises an agonist of a co-stimulatory molecule. In some embodiments, the agonist comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Agonists include agonistic antibodies, polypeptides, compounds, and nucleic acids.

### D. Dendritic Cell Therapy

In some aspects, a cancer therapy of the present disclosure comprises a dendritic cell therapy (also “dendritic cell vaccine”). Without wishing to be bound by theory, dendritic cell therapy is understood to provoke anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of dendritic cell therapy is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7. TLR8 or CD40 have been used as antibody targets.

A dendritic cell therapy may comprise a population of dendritic cells, which may include one or more types of dendritic cells. Types of dendritic cells that may be used in a dendritic cell therapy of the disclosure include, for example, conventional DCs (e.g., conventional type 1 dendritic cells (cDC1s), conventional type 1 dendritic cells (cDC2s)), plasmacytoid DCs, and monocytic DCs. A dendritic cell therapy may be autologous or allogeneic.

Various types of dendritic cell therapy are recognized in the art, including for example those described in Santos P M, Butterfield L H. Dendritic Cell-Based Cancer Vaccines. J Immunol. 2018; 200(2):443-449, incorporated herein by reference in its entirety.

### E. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell, NK cell, or other immune cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Example CAR-T therapies include Tisagenlecleucel (Kymriah) and Axicabtagene ciloleucel (Yescarta).

### F. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an example interleukin cytokine therapy.

### G. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein. For example, the patient may be one that has been determined to be sensitive to an immune checkpoint inhibitor therapy based on a determination that the patient has or previously had pancreatitis.

### II. Pancreatic Cancer Treatment

Aspects of the present disclosure are directed to methods comprising treatment of a subject having, or suspected of having, pancreatic cancer. In some embodiments, the pancreatic cancer is pancreatic ductal adenocarcinoma (PDAC). In certain embodiments, the disclosed methods comprise treating a subject who currently has or has previously had inflammation of the pancreas. Inflammation of the pancreas may include, but is not limited to, acute pancreatitis, chronic pancreatitis, organ damage (e.g., due to a bacterial infection), and fibrosis. A subject may be determined to have or have had inflammation of the pancreas by, for example, detecting the presence of CD11c⁺ cells in pancreatic tissue from the subject. In some embodiments, a subject is treated who concurrently has pancreatitis. For example, in some embodiments, a method comprises treating a subject having (e.g., experiencing symptoms of) PDAC where the subject currently has chronic pancreatitis. A subject may be diagnosed with pancreatitis using tests and diagnostic methods known in the art. For example, a subject may be determined to have pancreatitis by testing the subject for one or more symptoms of pancreatitis. In another example, a subject is determined to have pancreatitis by detecting an increased level of one or more pancreatic enzymes (e.g., amylase, lipase) in the subject relative to a control or healthy subject. In some embodiments, a subject is treated who previously had pancreatitis. For example, in some embodiments, a method of the disclosure comprises treating a subject having PDAC where the subject previously suffered and recovered from acute pancreatitis.

In some embodiments, the disclosed methods comprise treating a subject suffering from pancreatic cancer with a cancer immunotherapy. As disclosed herein, pancreatic cancer preceded by or associated with inflammation of the pancreas is surprisingly and unexpectedly sensitive to cancer immunotherapy. Accordingly, in some embodiments, disclosed is a method for treating a subject suffering from pancreatic cancer with a cancer immunotherapy, where the subject previously had or currently has inflammation of the pancreas, including pancreatitis. In some embodiments, the cancer immunotherapy is a dendritic cell therapy. In some embodiments, the cancer immunotherapy is an immune checkpoint blockade therapy (e.g., anti-PD-1 therapy, anti-CTLA4 therapy, etc.). In some embodiments, the cancer immunotherapy comprises a dendritic cell therapy and an immune checkpoint blockade therapy.

In some embodiments, the disclosed methods comprise identifying one or more subjects as being candidates for cancer immunotherapy treatment based on current or former pancreatitis. For example, in some embodiments, disclosed is a method comprising identifying a subject having pancreatic cancer as being a candidate for cancer immunotherapy by determining that the subject currently has or previously had pancreatitis. In some embodiments, the disclosed methods comprise determining an optimal cancer treatment for a subject with pancreatic cancer. For example, a subject may be given a cancer immunotherapy (e.g., dendritic cell therapy, immune checkpoint blockade therapy, adoptive cell therapy) if the subject has or previously had pancreatitis but given an alternative therapy (e.g., chemotherapy, radiation, hormone therapy, surgery) if the subject does not have or has not had pancreatitis. In some embodiments, a subject is given multiple types of cancer therapy, for example a cancer immunotherapy and a chemotherapy. In some embodiments, the disclosed methods comprise identifying one or more subjects as being candidates for cancer immunotherapy treatment based on the presence of CD11c⁺ cells in pancreatic tissue from the subject. In some embodiments, the disclosed methods comprise identifying one or more subjects as being candidates for cancer immunotherapy treatment based on the presence of tertiary lymphoid structures cells in pancreatic tissue from the subject.

Further aspects of the disclosure include methods for treatment of pancreatic cancer comprising administering immunotherapy (e.g., immune checkpoint blockade therapy) to a subject determined to have tertiary lymphoid structures in pancreatic cancer tissue. Various means of identifying tertiary lymphoid structures are known in the art and contemplated herein. For example, a subject may be administered an immunotherapy following identification of tertiary lymphoid structures by pathological and/or morphological analysis of tumor tissue from the subject. Subjects may be identified as being candidates for immunotherapy by the identification of tertiary lymphoid structures in pancreatic cancer tissue. Also contemplated are treatment methods comprising stimulating the formation of tertiary lymphoid structures in pancreatic tissue of a subject with pancreatic cancer, followed by treatment with immunotherapy (e.g., immune checkpoint blockade therapy). Any method for induction of tertiary lymphoid structure formation may be used in the disclosed methods.

### III. Administration of Therapeutic Compositions

The therapy provided herein may comprise administration of a combination of therapeutic agents, such as a first cancer therapy (e.g., dendritic cell therapy) and a second cancer therapy (e.g., immune checkpoint blockade therapy). The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IV. Kits

Certain aspects of the present invention also concern kits containing compositions of the invention or compositions to implement methods of the invention. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating biomarker activity in a cell.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments.

### EXAMPLES

The following examples are included to demonstrate embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1—Pancreatitis Activates Dendritic Cells and Sensitizes Pancreatic Ductal Adenocarcinoma to Immunotherapy

Inflammation associated with pancreatitis increases the risk for development and progression of pancreatic ductal adenocarcinoma (PDAC). Existing literature presents a contradicting picture on the role of T cells in the progression of PDAC (15, 18, 19). One study that depleted CD4⁺ and CD8⁺ T cells in PDAC mice concluded that T cells had no role in tumor progression(15), whereas another study that analyzed tumor initiation in a pancreatitis induced model concluded that CD4⁺ T cells (18, 19) and in particular the Th17 subset (19) promote tumorigenesis in PDAC. Further, contradictory conclusions exist on the role of the regulatory T cells (Tregs) in PDAC(20, 21). One study that used a pancreatitis induced model concluded that Tregs restrain tumor initiation(21), whereas another study that employed an orthotopic system concluded that Tregs promote PDAC progression(20).

In the present disclosure, the inventors show that acute pancreatitis (AP) and chronic pancreatitis (CP) significantly activated antigen presenting dendritic cells (DCs) when compared to spontaneous pancreatic cancer mice. T cells had no impact on tumorigenesis and survival in spontaneous pancreatic cancer mice, whereas CD4⁺ T cells promote tumorigenesis in mice with pancreatitis. Depletion of CD4⁺ T cells in concurrence with pancreatitis led to attenuation of pancreatic cancer, which was reversed by blocking the function of CD11c⁺ DCs. The CD4⁺ T cells promote tumorigenesis in mice with pancreatitis by restraining activated DCs. Recruitment of activated dendritic cells via. chronic pancreatitis or administration of conventional dendritic cell 1 (cDC1) vaccine rendered immunotherapy resistant PDAC sensitive to checkpoint immunotherapy resulting in activation of cytotoxic CD8⁺ T cells and dramatic increase in overall survival with cures. The DC infiltration in human PDAC correlates with infiltration of CD8⁺ T cells and predicts longer disease specific survival (DSS). The inventors' findings reveal fundamental differences in the immune regulation of PDAC with and without underlying pancreatitis and offers rationale for combining cDC1 vaccine and checkpoint immunotherapy. The inventors' studies suggest that PDAC patients with higher DC infiltration or a history of chronic pancreatitis could benefit from checkpoint immunotherapy.

### Results

### Pancreatitis Accelerates Tumor Initiation and Recruits Activated Dendritic Cells

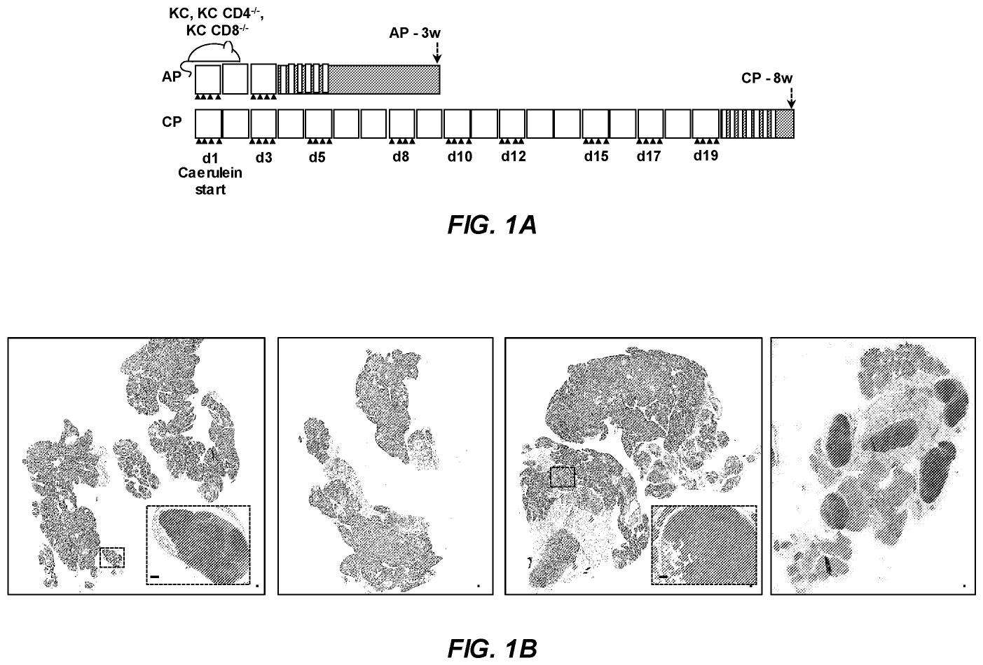

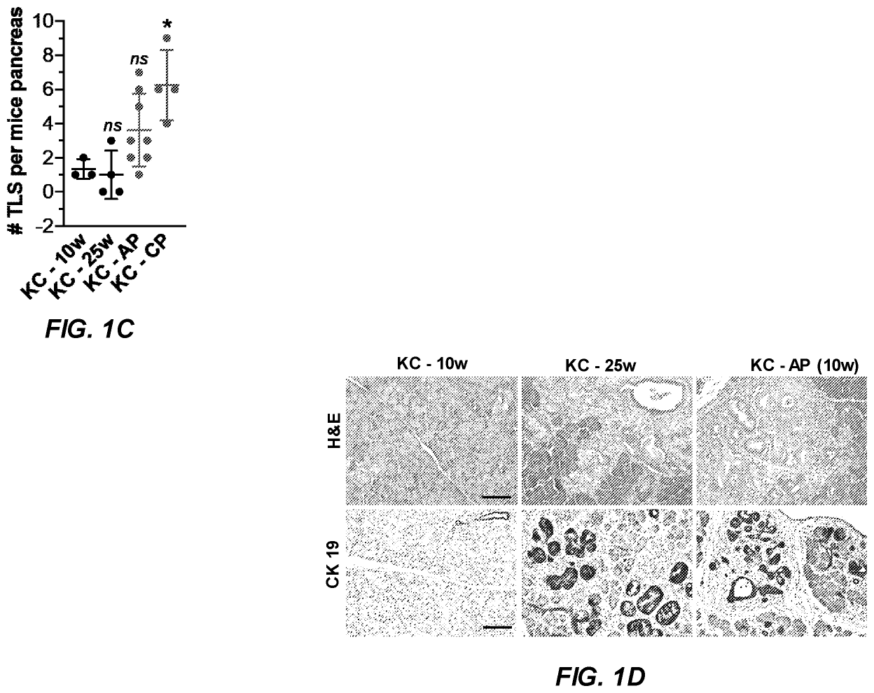

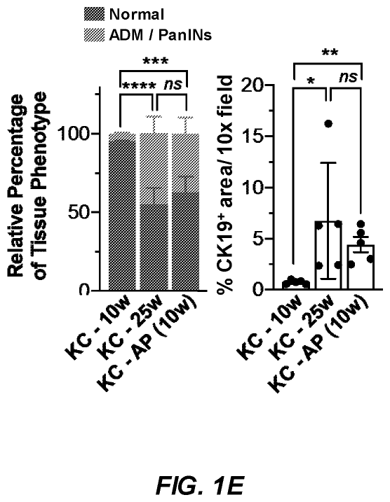

To analyze the impact of pancreatitis on tumor initiation, the inventors induced AP in 7w old KC (Pdx1-Cre; LSL-Kras^{G12D/+}) mice and sacrificed these mice 21 days after AP induction for histological analysis (FIG. 1A). Age matched KC mice (˜10w) were used as controls for analysis of tumor initiating pancreatic intraepithelial neoplasia (PanIN) lesions. CP resulted in a significant increase in tertiary lymphoid structures (TLS) although an increasing trend was observed with AP in the pancreatic tissue (FIGS. 1B, C).

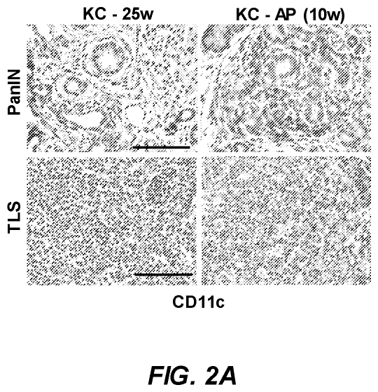

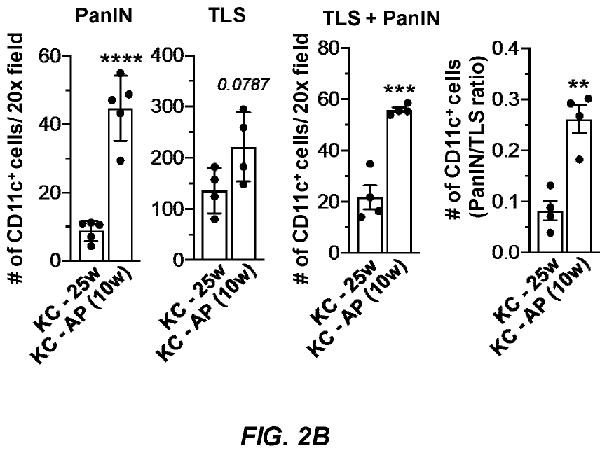

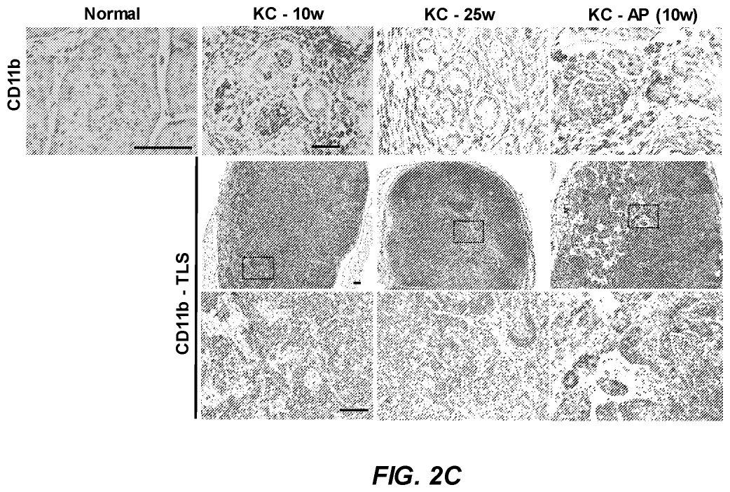

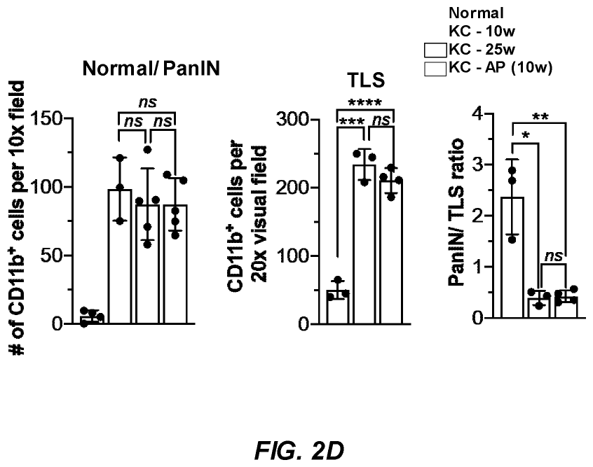

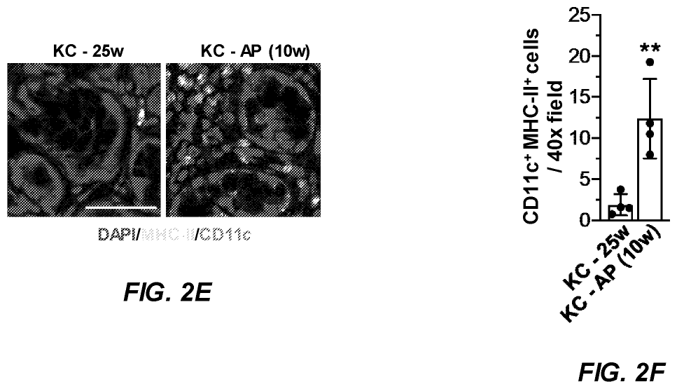

AP resulted in acceleration of tumor initiation in KC mice as seen by histological phenotypes and cytokeratin 19 (CK19) staining (FIGS. 1D, E). As the age matched KC mice at 10w did not have robust PanIN lesions, the inventors then analyzed KC mice at a later time point (at 25w of age). The older KC mice at 6m had similar percentage of PanIN lesions as the AP induced KC mice (FIGS. 1D, E), further supporting the inventors' finding that AP results in acceleration of tumor initiation. The inventors use the 25w old KC mice for analysis of differences in the immune microenvironment induced by pancreatitis as they had similar stage of tumor initiation with the AP induced mice. Similar to findings in earlier studies in wildtype mice (22), AP in KC mice increased the number of CD11c⁺DCs in both the PanIN lesions and the associated TLS taken together (FIGS. 2A, B). TLS harbored the majority of the baseline level of CD11c⁺ cells, the PanIN lesions barely showed any positivity for CD11c staining. AP resulted in a significant increase in the CD11c⁺ DCs in PanIN lesions, whereas only a modest increase was observed in the TLS (FIGS. 2A, B). Taken as a ratio of CD11c⁺ cells between the PanIN lesions and TLS, the PanIN lesions of the KC mice with AP demonstrated a significantly higher number of DCs (FIGS. 2A, B). Further analysis of myeloid marker CD11b immunostainings revealed that the CD11b⁺ myeloid cells increased significantly in the PanIN lesions compared to the uninvolved normal pancreas (FIGS. 2C, D). In the TLS, the myeloid infiltration increased with AP when comparing age matched mice (FIGS. 2C, D), whereas older stage matched KC mice showed similar levels of CD11b infiltration indicating that myeloid infiltration increased with both pancreatitis and tumor progression (FIGS. 2C, D). Both AP and older stage matched KC mice had a higher number of CD11b⁺ myeloid cells compared to the normal pancreas and presence of pancreatitis by itself did not increase the myeloid cells in the PanIN lesions (FIGS. 2C, D). Further analysis of the CD11c⁺ DC compartment with co-staining for activation marker MHC-II and CD11c revealed that AP recruited MHC-II⁺ CD11c⁺ DCs in the PanINs of KC mice (FIGS. 2E, F).

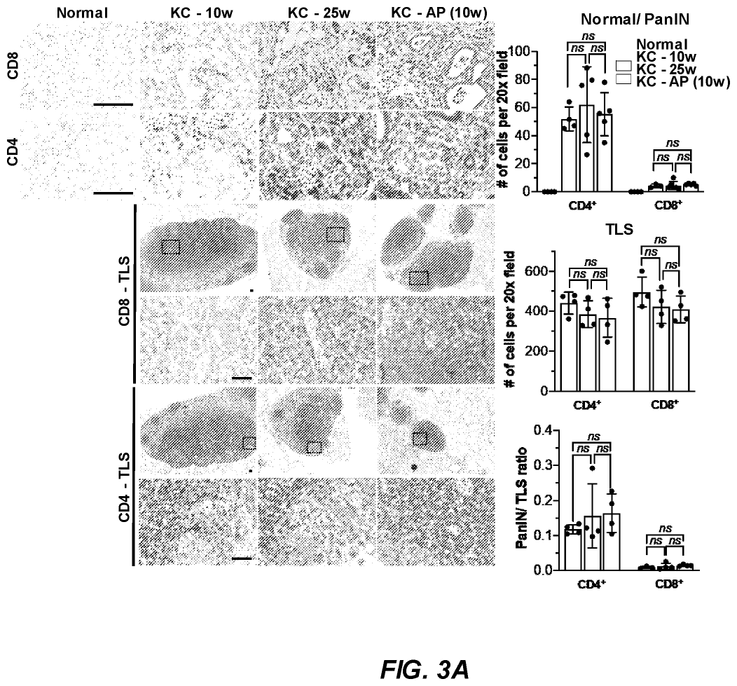

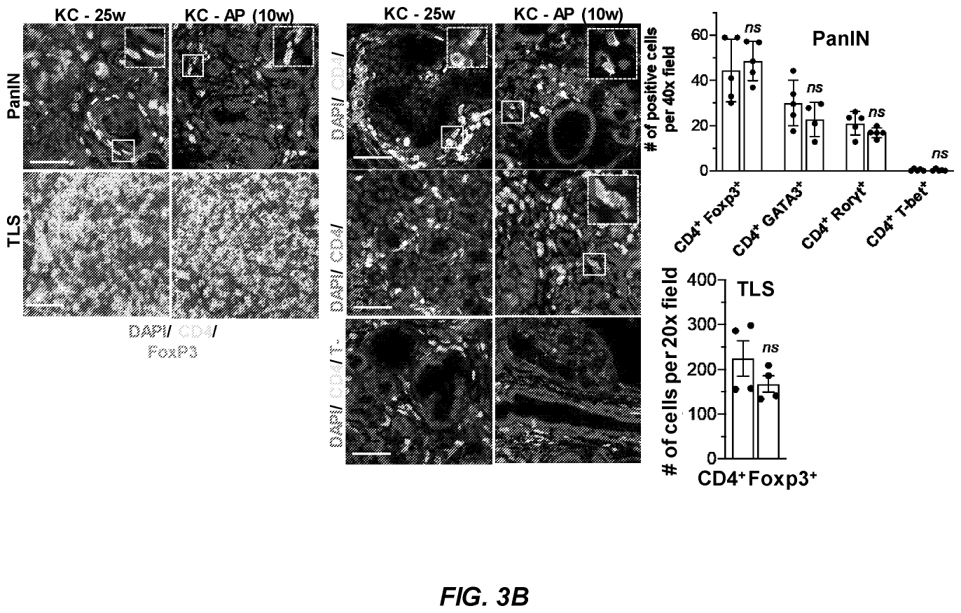

Further, analysis of T cell populations by immunostaining demonstrated no significant differences in CD4⁺ and CD8⁺ T cells in the PanINs and TLS of KC mice with and without pancreatitis (FIG. 3A). The inventors' analysis indicated the PanIN lesions had an abundance of CD4⁺ T cells which led the inventors to further characterize its different subsets. Analysis of the different CD4⁺ T cell subsets by expression of nuclear transcription factors (CD4⁺ Foxp3⁺: Treg, CD4⁺ GATA3⁺: Th2 cells, CD4⁺Rorγt⁺: Th17 cells and CD4⁺T-bet⁺: Th1 cells) revealed no differences between KC mice with and without AP (FIG. 3B). Analysis of different T cell populations in the TLS revealed no differences in CD4⁺, CD8⁺ and CD4⁺ Foxp3⁺ cells between the KC mice with and without pancreatitis (FIG. 3B).

T Cells have No Impact on Tumorigenesis and Survival in PDAC, Whereas CD4⁺ T Cells Promote Tumorigenesis in Mice with Pancreatitis

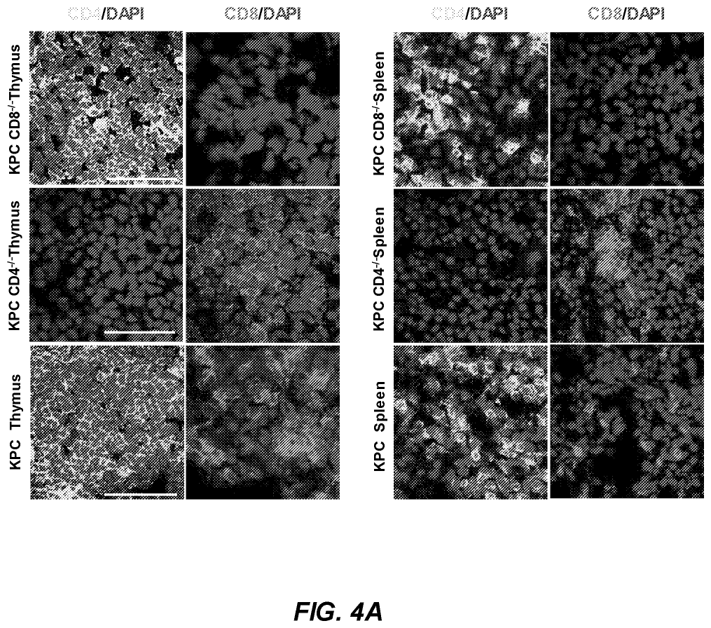

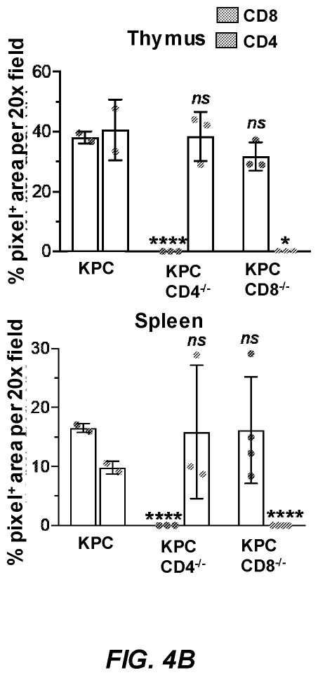

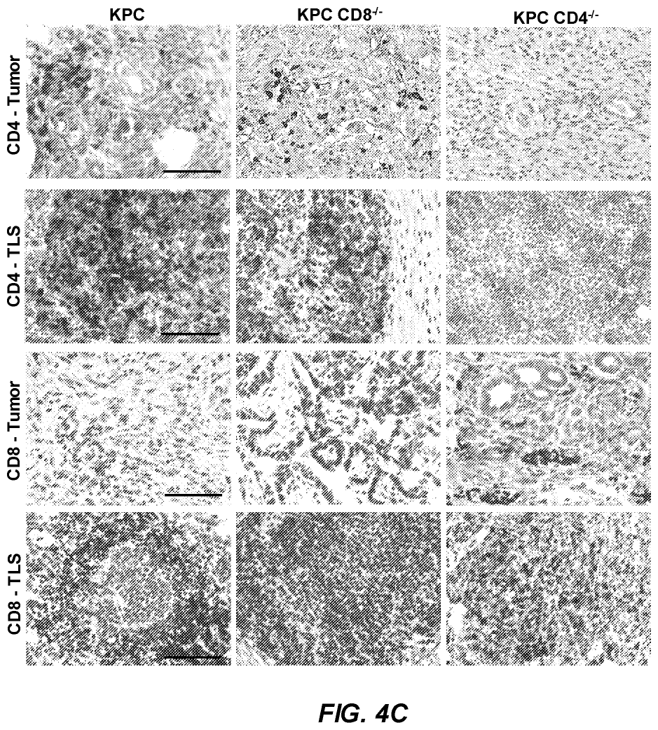

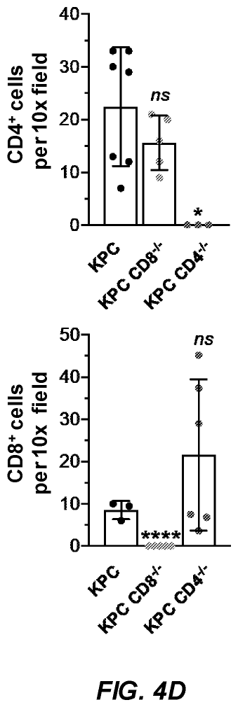

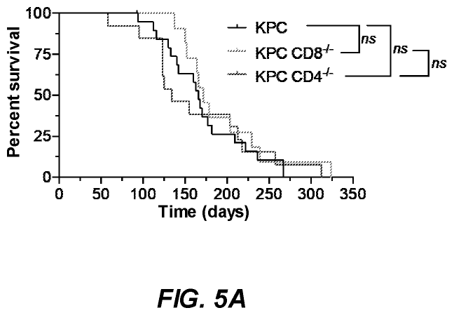

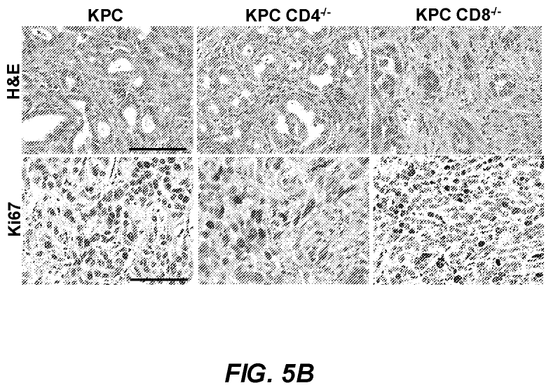

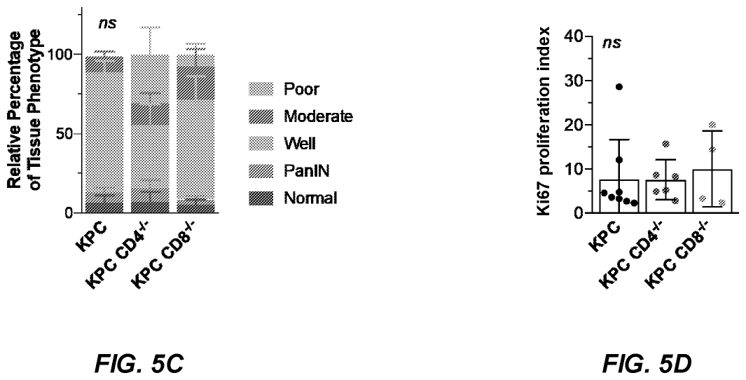

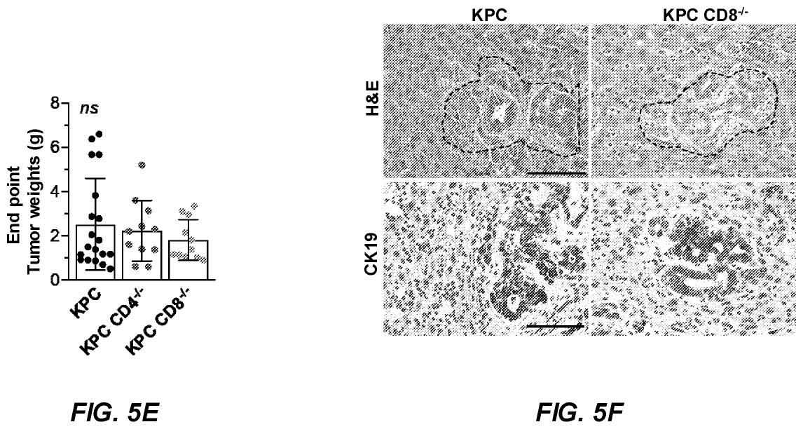

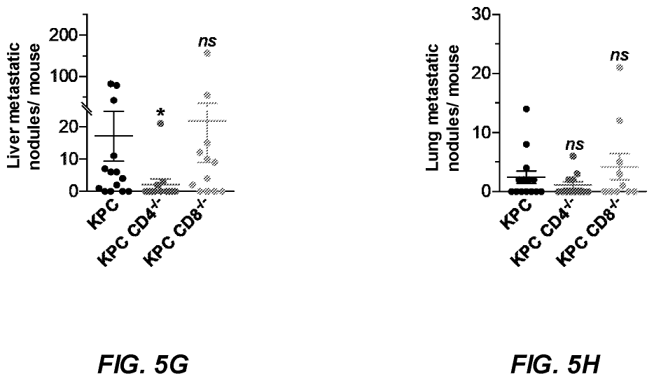

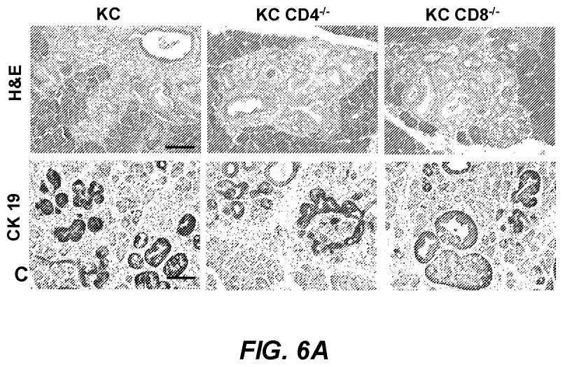

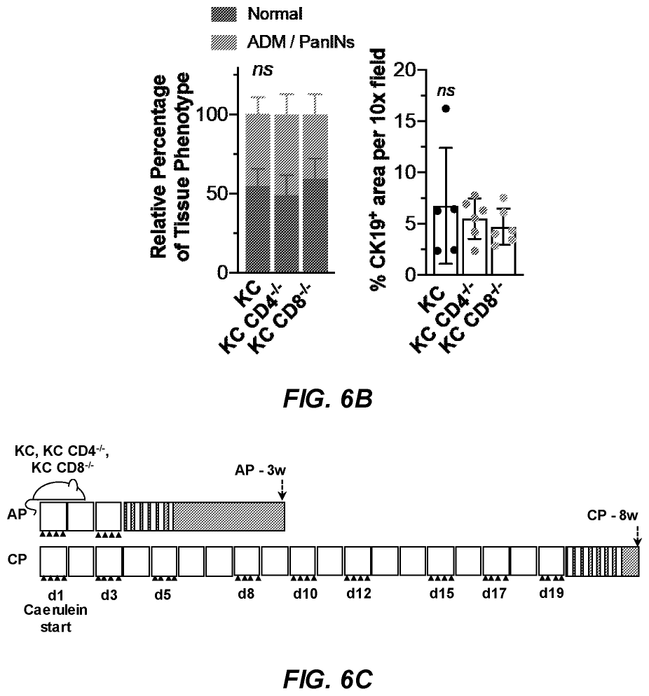

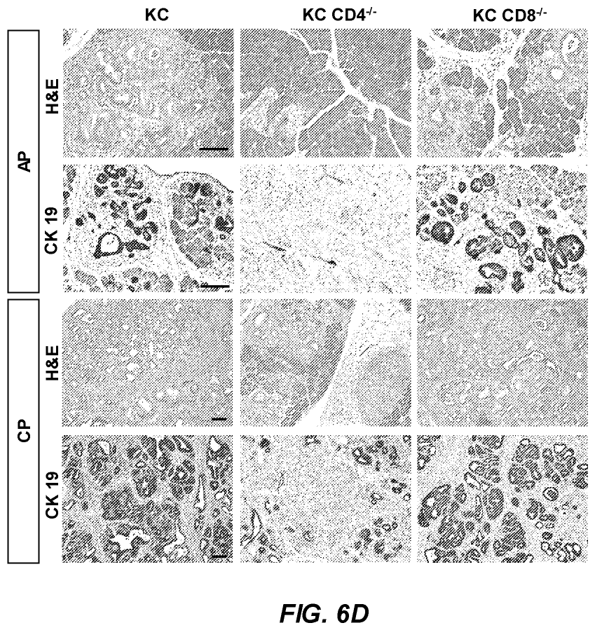

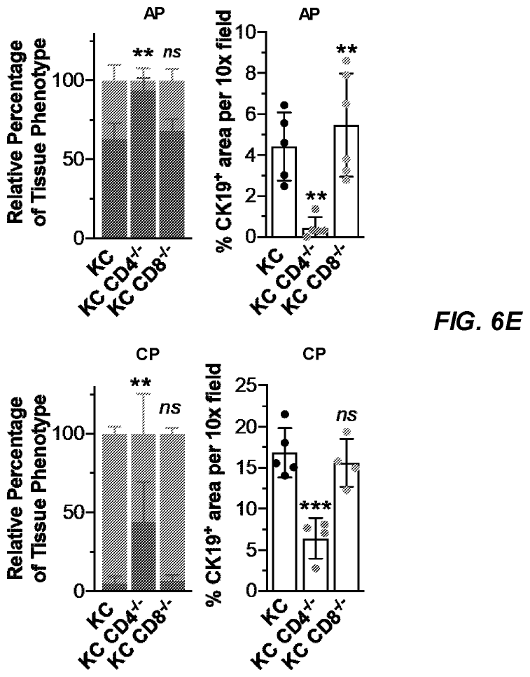

The inventors next examined the impact of CD11c⁺ DCs recruited by pancreatitis on T cell function and probe whether the cross talk between DCs and distinct T cell populations is of therapeutic relevance in PDAC. To understand the distinct functions of T cells in tumors with and without underlying pancreatitis, the inventors analyzed tumor initiation and progression in mice with genetic depletion of CD4⁺ or CD8⁺ T cell populations. The inventors crossed CD4^{-/-} or CD8^{-/-} mice with Pdx1-Cre; LSL-Kras^{G12D/+}; P53^{R172H/+}(KPC) and generated CD4^{-/-}; Pdx1-Cre; LSL-Kras^{G12D/+}; P53^{R172H/+}(KPC CD4^{-/-}) and CD8^{-/-}; Pdx1-Cre; LSL-Kras^{G12D/+}; P53^{R172H/+}(KPC CD8^{-/-}) mice. Depletion of CD4⁺ and CD8⁺ T cells in the thymus, spleen, and tumors of KPC CD4^{-/-} and KPC CD8^{-/-} mice was confirmed by immunolabeling (FIGS. 4A-D). The KPC CD4^{-/-} and KPC CD8^{-/-} mice developed PDAC tumors and showed a similar median survival as the control KPC mice (FIG. 5A). Analysis of tumor histology, tumor weights and Ki67 proliferation index did not reveal any differences between the CD4, CD8 knockout and control KPC mice (FIGS. 5B-E). Although depletion of CD4⁺ or CD8⁺ T cells did not affect primary tumor growth, KPC CD4^{-/-} mice had attenuated liver metastasis compared to KPC mice, whereas the depletion of CD8⁺ T cells did not affect metastasis (FIGS. 5F, H). Depletion of CD4⁺ or CD8⁺ T cells did not alter lung metastasis in KPC mice (FIG. 5G). This finding is consistent with earlier studies with breast cancer metastasis models that deplete CD4⁺ T cells(23). The study demonstrated that CD4⁺ T cells indirectly promote metastasis by regulation of tumor associated macrophages(23). Next, the inventors performed age matched analysis (at 25w) of tumor initiation in the KC, KC CD4^{-/-} and KC CD8^{-/-} mice. Analysis of PanIN lesions in these three cohorts of mice showed no significant differences in tumor initiation as seen by histological phenotypes or CK19 immunostaining (FIGS. 6A, B). None of the KC mice in the inventors' cohort demonstrated evidence of invasive cancer in the inventors' cohort. Therefore, CD4⁺ or CD8⁺ T cells did not impact tumorigenesis, primary tumor growth and survival in PDAC mice without pancreatitis.

To understand the influence of underlying pancreatitis on pancreatic cancer initiation, AP and CP were induced in 7w old KC, KC CD4^{-/-} and KC CD8^{-/-} mice with caerulein injections and these mice were sacrificed 3w (for AP) and 8w (for CP) later to assess tumor initiation (FIG. 6C) (19). In contrast to tumor initiation in mice without pancreatitis, the inventors observed that KC CD4^{-/-} mice with pancreatitis had relatively fewer PanIN lesions compared to KC and KC CD8^{-/-} mice (FIGS. 6D, E). To assess the robustness of the findings, the inventors probed the role of CD4⁺ T cells in tumor initiation in a setting of underlying chronic pancreatitis (CP). These mice were sacrificed 8 weeks following induction of pancreatitis to assess tumor histology. Consistent with the inventors' findings in KC mice with AP, CD4⁺ T cells promoted tumorigenesis in KC mice with CP. KC CD4^{-/-} mice had fewer PanIN lesions compared to KC and KC CD8^{-/-} mice as seen by tumor histology and CK 19 immunostaining (FIGS. 6D, E). CD8⁺ T cells did not have any impact on tumor initiation in KC mice with or without pancreatitis.

CD4⁺ T Cells Promote Tumorigenesis in KC Mice with Pancreatitis by Restraining Dendritic Cells

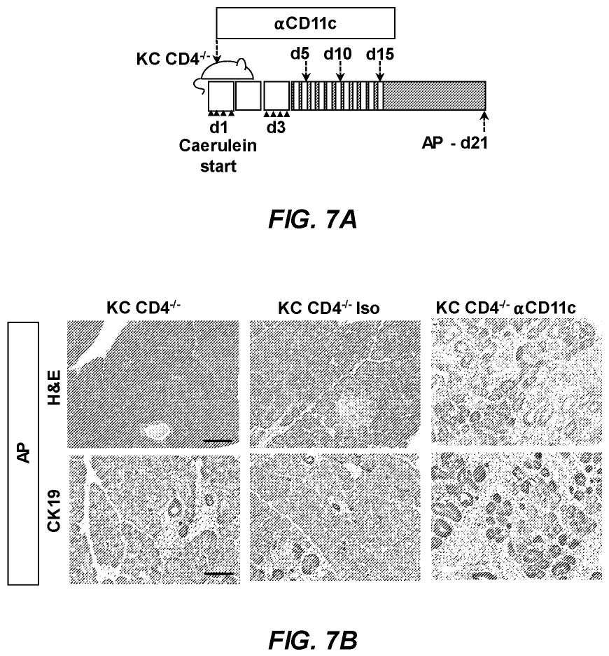

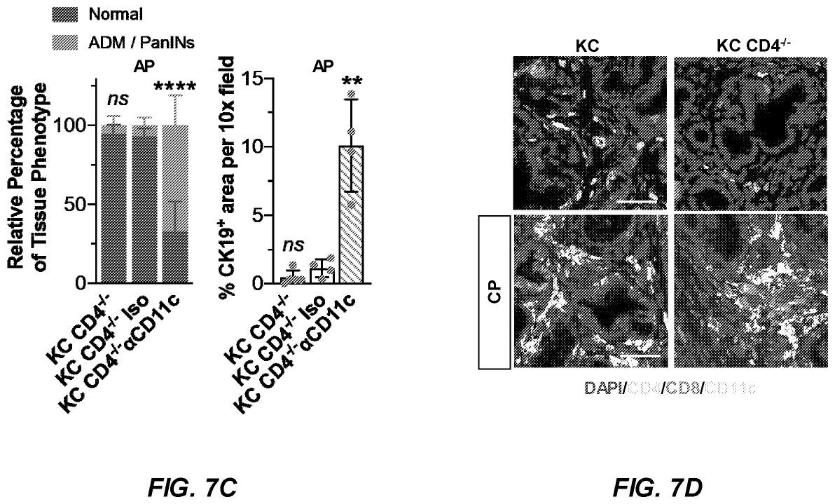

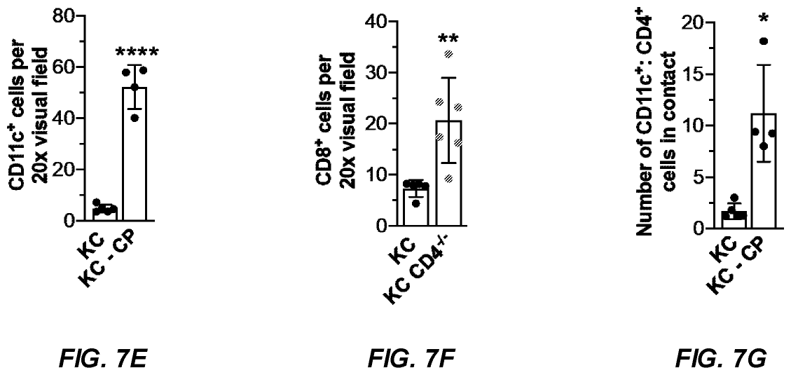

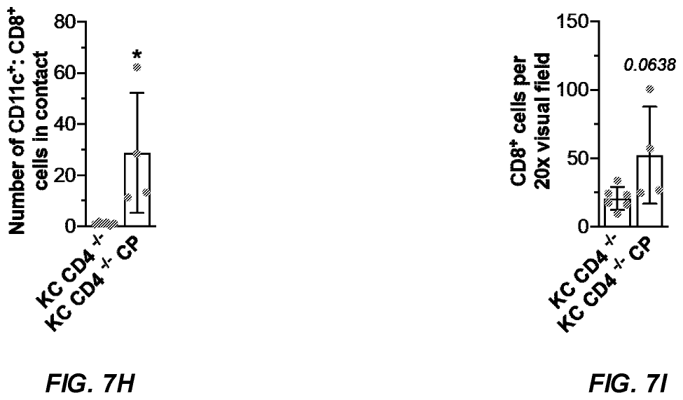

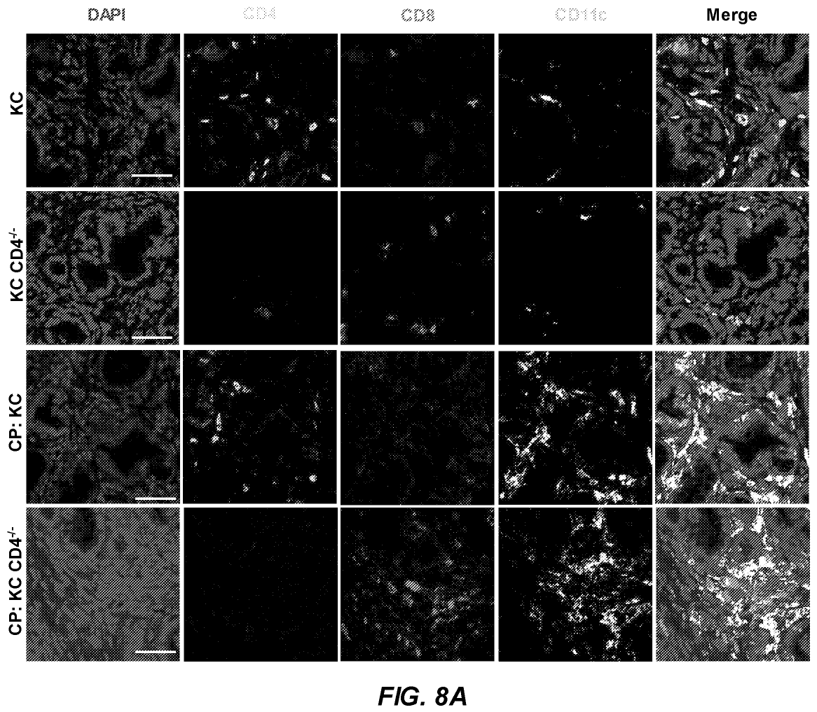

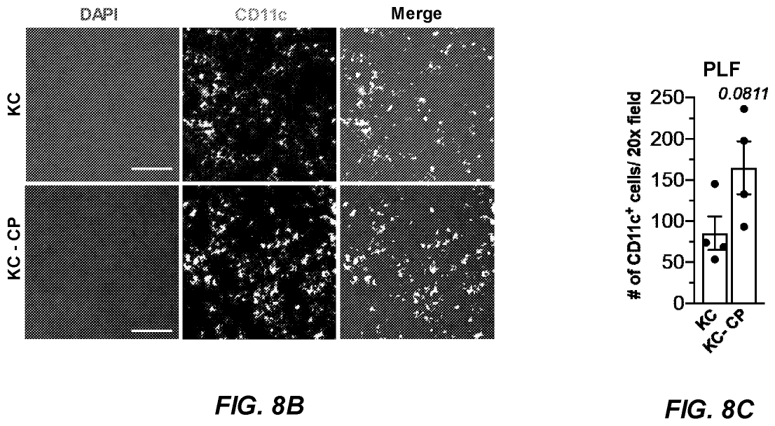

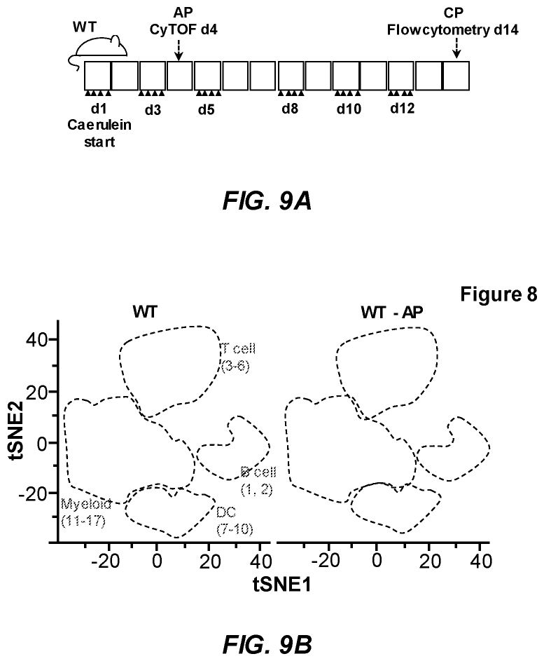

Since the KC CD4^{-/-} mice demonstrated inhibition of tumor initiation in pancreatitis induced mice, the inventors hypothesized that CD4⁺ T cells inhibited activated DCs, and consequently the depletion of CD4⁺ T cells led to inhibition of tumor initiation. In the absence of DCs, neither CD4⁺ nor CD8⁺T cells played any tumor restricting or promoting role in PDAC progression. To understand the crosstalk between CD4⁺ T cells and CD11c⁺ DCs in pancreatitis induced mice, the inventors treated KC CD4^{-/-} mice with αCD11c antibody to determine if depletion of dendritic cells would rescue tumor inhibition (FIG. 7A). Depletion of CD11c⁺ DCs in AP induced KC CD4^{-/-} mice rescued tumor initiation (FIGS. 7B, C), indicating that CD4⁺ T cells inhibit DCs to promote tumor initiation. Further, the inventors analyzed the crosstalk between CD4⁺ T cells and DCs in the chronic pancreatitis induced KC and KC CD4 mice. Since the AP induced KC CD4^{-/-} mice had very few PanIN lesions, the inventors utilized the CP model for further analysis of T cell—DC interactions. Similar to the inventors' phenotype in AP induced KC mice, CP increased the number of CD11c⁺ DCs (FIGS. 7D, E, 8A). Although, no difference in tumor initiation was observed between the KC and KC CD4^{-/-} mice, there was an increase in the number of CD8⁺ T cells in the KC CD4^{-/-} mice (FIG. 7F). However, due to lack of antigen presenting DCs in the PanINs developing without pancreatitis, increase in CD8⁺ T cells did not result in inhibition of tumor initiation in the KC CD4^{-/-} mice as seen earlier (FIGS. 6A, B). Although pancreatitis by itself did not change the T cell composition in PanIN lesions, KC mice with CP showed an increased contact between CD11c⁺ DCs and CD4⁺ T cells exert an inhibitory influence on the CD11c⁺ DCs (FIG. 7G). Depletion of CD4⁺ T cells removed its inhibitory effect on DCs, thereby increasing CD11c: CD8⁺ T cell interaction resulting in tumor growth inhibition in the presence of pancreatitis (FIGS. 7H, I). In the KC CD4^{-/-} chronic pancreatitis mice, the inventors observed increased interaction between the CD11c⁺ DCs and CD8⁺ T cells, whereas the KC counterparts with CP exhibited interaction between the CD11c⁺ DCs and the inhibitory CD4⁺ T cells (FIGS. 7D, G, H). Overall, the results indicated that both acute and chronic pancreatitis recruit dendritic cells to the pancreas of KC mice. Despite the increase in antigen presentation, a preponderance of inhibitory CD4⁺ T cells override the effect of DCs in KC mice resulting in tumor progression in the presence of pancreatitis. Depletion of CD4⁺ T cells in pancreatitis induced mice enables antigen presentation by DCs to the CD8⁺ T cells resulting in inhibition of tumor initiation in the KC CD4^{-/-} mice.

Characterization of Pancreatic Immune Infiltrates of Wildtype and PDAC Mice with Pancreatitis

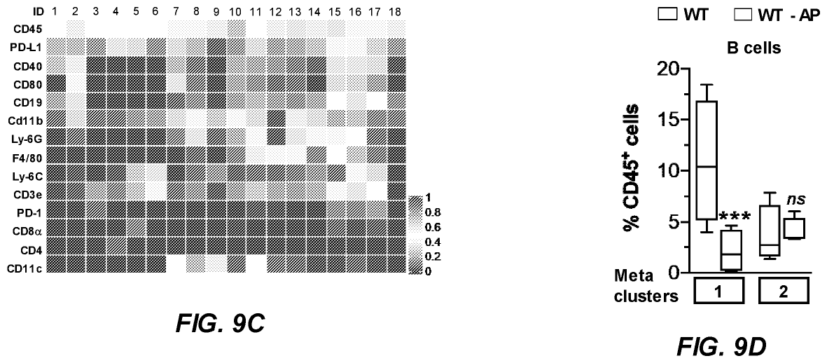

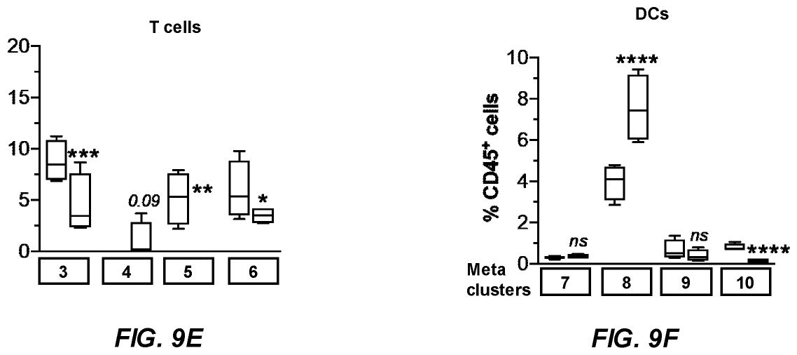

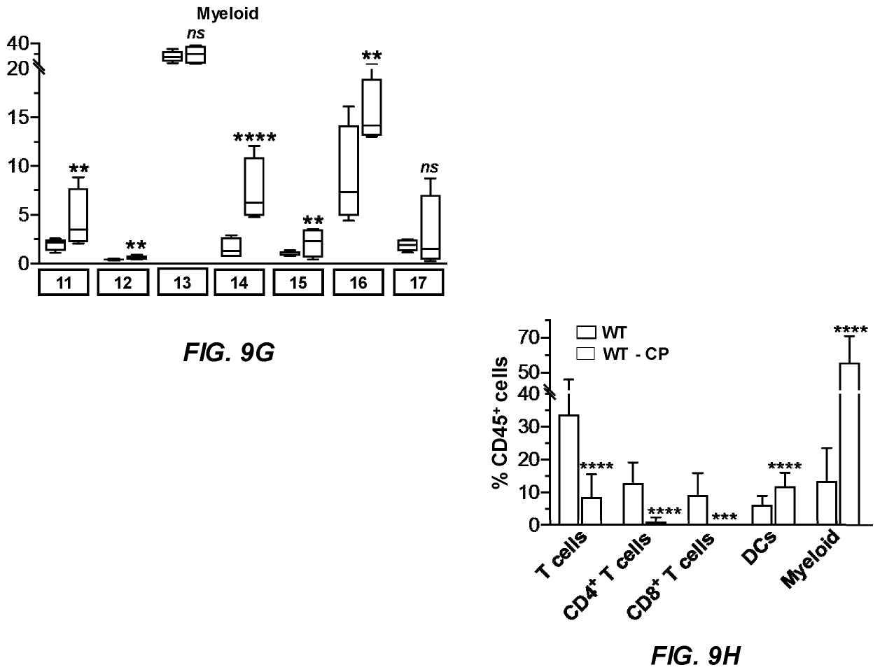

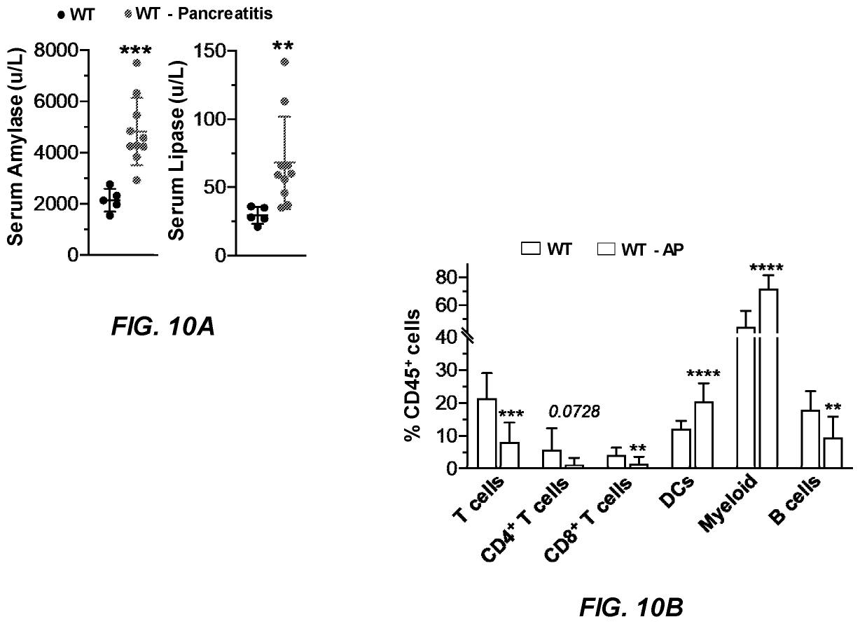

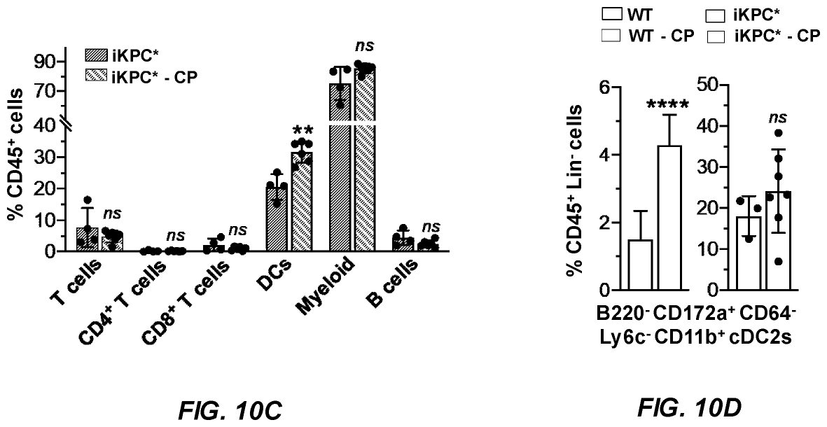

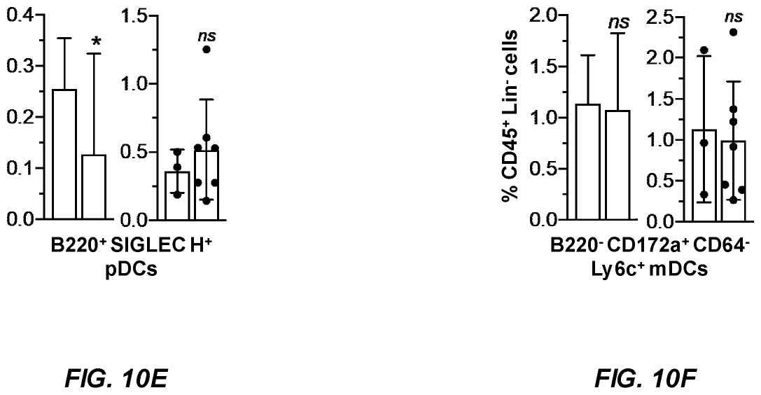

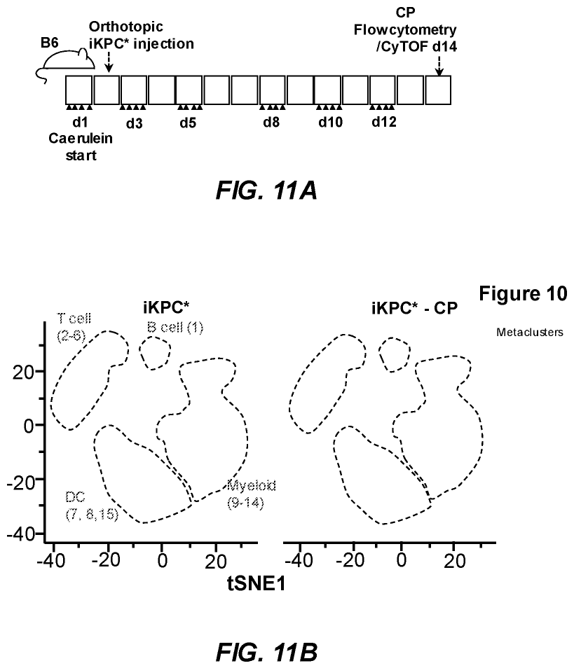

To analyze the impact of pancreatitis on the immune infiltrates of normal pancreas, the inventors induced acute and chronic pancreatitis with injections of caerulein (an analogue of cholecystokinin implicated in the pathogenesis of pancreatitis) as described previously (18, 19) in wildtype (WT) mice (FIG. 9A). Consistent with induction of pancreatitis, the inventors observed an increase in serum amylase and lipase in caerulein injected WT mice compared to controls seven days following pancreatitis induction (FIG. 10A). The inventors utilized flow cytometry or mass cytometry (CyTOF) based systems approach to identify immune populations on day 4 (for acute pancreatitis—AP) and on day 14 (for chronic pancreatitis—CP) after start of caerulein injections. In WT mice with AP. FlowSOM analysis on CyTOF data with unsupervised hierarchical cluster on CD45+ cells (manually gated) using R software identified 18 metaclusters (MCs) which were grouped into B cell (MC 1-2), T cell (MC 3-6), DC (MC 7-10) and myeloid (MC 11-17) populations (FIGS. 9B-G). For classification of CD11c⁺ cells into DC and myeloid MCs, F4/80⁻ Ly-6G⁻ CD11c⁺ cells were classified as DC MCs and F4/80⁺ or Ly-6G⁺ CD11c⁺ cells were grouped into myeloid MCs, viSNE plots on these 18 MCs in WT mice and WT-AP mice are shown in FIG. 9B. Among the T and B cell populations, AP resulted in a decrease in the proportion of CD40⁺ CD19⁺ B cells (MC1) (FIGS. 9B-D), and in CD4⁻ and CD8⁻ negative (CD3⁺ CD4⁻ CD8⁻T-MC3, CD3⁺ CD4⁻ CD8⁻ Ly-6C⁺-MC6) T cells, and CD45⁺ CD3⁺ CD8⁺ T (MC5) (FIGS. 9B-E). However, AP did not result in any significant differences in the CD3⁺CD4⁺ T (MC4) cells although a decreasing trend was observed with AP. Among the DC populations, AP resulted in an increase in the proportion of CD11b^{int} CD11c⁺ (MC8) DCs (FIGS. 9B-F). Further, AP resulted in an increase in the proportion of CD11b⁺ Ly-6G⁺ F4/80⁺ CD11c⁺ (MC11), CD11b⁺ Ly-6G⁺ CD11c⁺ (MC12), CD11b⁺ Ly-6G^{int} Ly-6C⁺ (MC14) and CD11b⁺Ly6-G⁺ F4/80⁺ (MC16) myeloid populations (FIGS. 9B-G). AP did not result in differences in B cells MCs (MC2), DC MCs (MC 7, 9) and myeloid MCs (MC 13, 15, 17) (FIGS. 9D-G). However, a decrease in proportion of one of the minor DC populations PD-L1⁺ CD40⁺CD80⁺CD11b^{int} CD11c⁺(MC10) was seen in AP mice (FIG. 9F). Immunophenotyping analysis by Boolean gating of the CyTOF data also confirmed that AP resulted in a decrease in the frequency of T cells (CD3⁺), CD8⁺ T cells (CD3⁺CD8⁺), B cells (CD19⁺CD3⁻), accompanied by an increase in myeloid (CD11b⁺) and DCs (CD11c⁺) (FIG. 10B). Although there was a trend towards a decrease in CD4⁺ T cells (CD3⁺ CD4⁺) with AP, this was not statistically significant (FIG. 10B). To confirm the robustness of the findings in AP, the inventors also performed immunophenotyping analysis of the pancreatic immune infiltrates in WT mice with chronic pancreatitis following caerulein injection by flowcytometry. Similar to the phenotype observed in AP mice, CP also resulted in a decrease in the proportion of T cells, CD4⁺ and CD8⁺ T cells, accompanied by an increase in myeloid and DC populations (FIG. 9H).

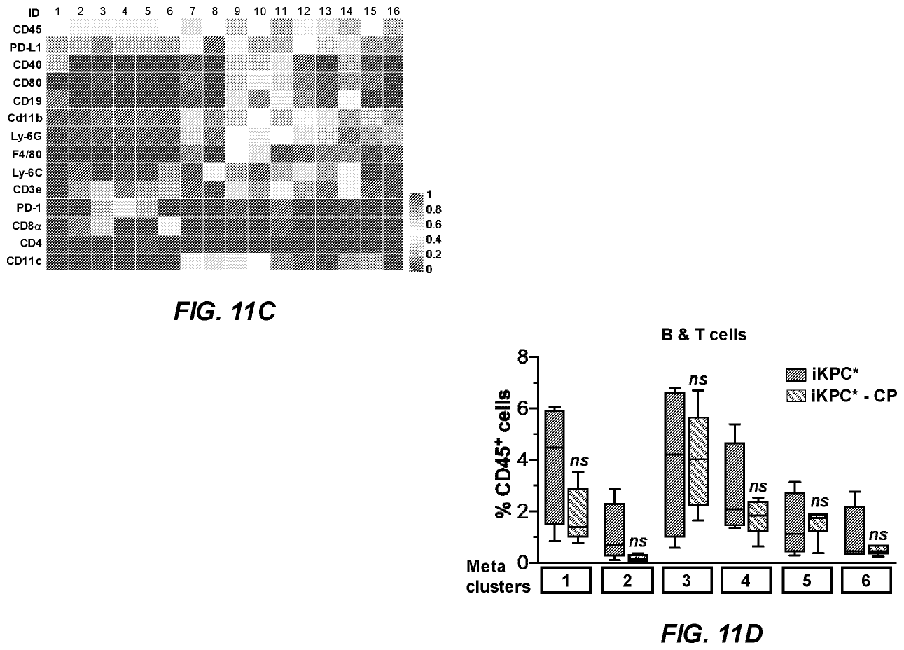

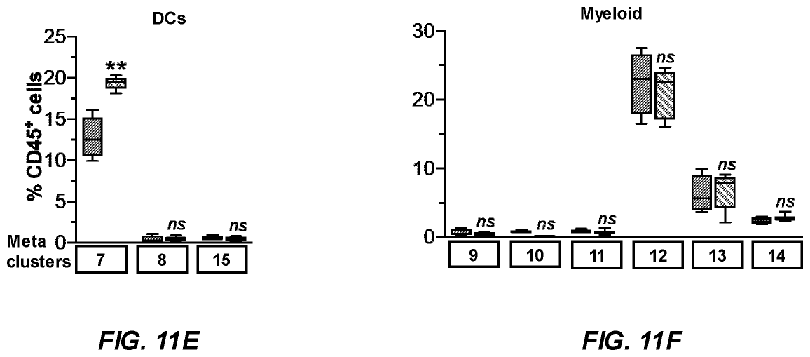

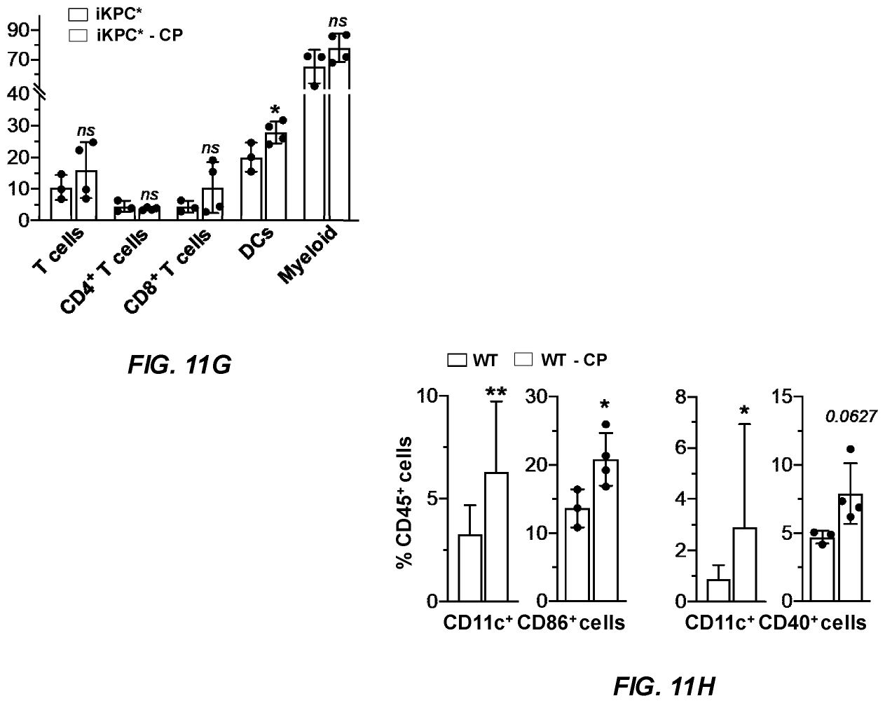

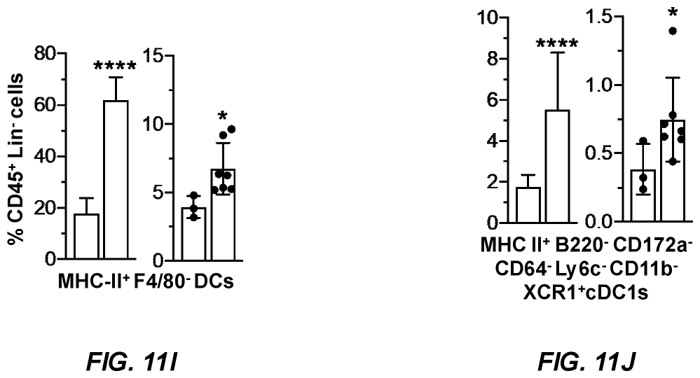

Next, the inventors analyzed the pancreatic immune infiltrates of orthotopic iKPC* (P48-Cre; tetO-LSL-Kras^{G12D/+}; P53^{L/L}) tumor bearing mice with CP (FIG. 11A). The pancreas specific expression of Kras^{G12D/+} in the tetracycline inducible iKPC* mice was maintained by feeding doxycycline in drinking water throughout the course of the inventors' experiments. For CP induction, the inventors consistently induce pancreatitis for 3 weeks as described previously (19) and subsequently analyze tumor initiation and survival for all the inventors' models. However, for analysis of immune infiltrates, the inventors chose the 2 week time point as a portion of iKPC* mice reached end point earlier than 3 weeks. FlowSOM analysis on CyTOF data with unsupervised hierarchical cluster identified 16 metaclusters (MCs) which were grouped into B cell (MC 1), T cell (MC 2-6), DC (MC 7, 8, 15) and myeloid (MC 9-14) populations (FIGS. 11B-G). viSNE plots on these 16 MCs are shown in FIG. 11B. CP in iKPC* mice resulted in an increase in proportion of CD45⁺ CD11c⁺ MC (MC7), whereas no significant differences in B cell, T cell and myeloid MCs were seen (FIGS. 11B-G). Immunophenotyping analysis of CyTOF and flowcytometry data in iKPC* mice with CP also revealed an increase in proportion of CD11c⁺ DCs, whereas no changes in T cells, CD4⁺ T cells, CD8⁺ T cells. B cells and myeloid cells were seen (FIGS. 11G, 10C).

Now that the inventors established that pancreatitis recruited CD11c⁺ DCs in WT and iKPC* orthotopic mice, the inventors further characterized the CD11c⁺ population accompanying CP in the WT and iKPC* mice. The inventors examined if these CD11c⁺ DCs expressed activation markers such as CD86 and CD40. Immunophenotyping analysis of flowcytometry data revealed that CP increased the proportion of CD86⁺ CD11c⁺ DCs both WT and iKPC* mice and showed an increasing trend in CD40⁺ CD11c⁺ DCs in iKPC* mice (FIG. 11H). Although pancreatitis induces an increase in the percentage CD11c⁺ cells, frequently, murine macrophages also express dendritic cell markers such as CD11c and MHC-II (24). Therefore, the inventors confirmed that pancreatitis resulted in an increase in MHC-II⁺ F4/80⁻ CD11c⁺ cells (gated as % CD45+ Lin (CD3, NK1.1, Ly-6G, CD19)⁻ cells) in both WT and iKPC* orthotopic mice (FIG. 11I). Recent characterization of DCs revealed distinct function, expression profiles and properties for different DC subsets and classify them as conventional DCs (cDCs), plasmacytoid DCs (pDCs) and monocytic DCs (mDCs)(24-26). Among the conventional DCs, the cDC1s are important in inducing antigen specific T cell responses to restrain tumors, whereas cDC2s are involved in tolerogenic immune responses that facilitate tumor growth(25). The inventors characterized cDC1s as CD45⁺ Lin⁻ CD11c⁺ B220⁻ CD172a⁻ CD64⁻Ly-6C⁻ CD11b⁻ MHC II⁺ XCR1⁺ cells and cDC2s as CD45⁺ Lin⁻CD11c⁺ B220⁻ CD172a⁺ CD64⁻ Ly-6C⁻ CD11b⁺ cells. pDCs are critical in type I interferon response and can develop into antigen presenting cells to activate T cells, whereas mDCs are generated in inflammation and autoimmune pathologies (24). The inventors characterized pDCs as CD45⁺ Lin⁻ CD11c⁺ B220⁺ SIGLEC H⁺ and mDCs as CD45⁺ Lin⁻ CD11c⁺ B220⁻ CD172a⁺CD64⁻ Ly-6C⁺ cells (24). Analysis of cDCs in WT and iKPC* mice showed that CP resulted in an increase in proportion of cDC1s in both WT and iKPC* mice (FIG. 11J). Although CP resulted in an increase in proportion of cDC2s and decrease in pDCs in WT mice, iKPC* mice did not demonstrate any significant differences in cDC2s (FIGS. 10D, E). No differences in mDCs were seen in both WT and iKPC* mice with CP (FIG. 11G).

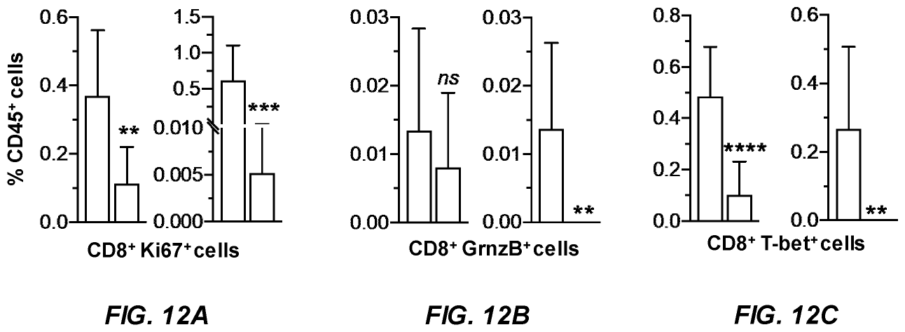

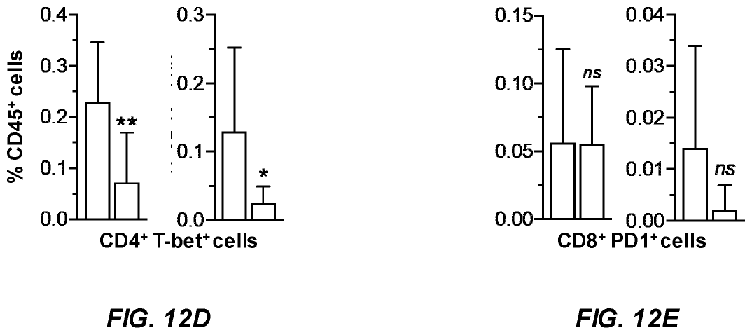

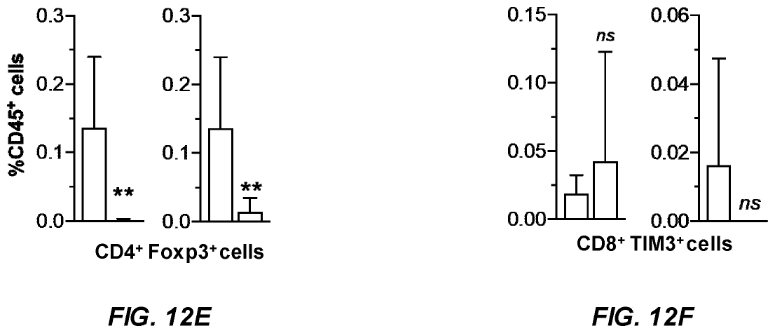

The characterization of the pancreatic immune infiltrates indicated that pancreatitis recruits CD11c⁺ DCs in both WT and iKPC* orthotopic mice. Although myeloproliferation and T cell suppression were seen in WT mice with pancreatitis, tumor bearing mice did not demonstrate any differences in these populations. Further analysis of the T cells in WT mice revealed a decrease in proportion of proliferating CD8⁺ T cells in mice with AP and CP (FIG. 12A). The proportion of CD8⁺ GrnzB⁺ cells did not change in mice with AP but showed a significant decrease in mice with CP (FIG. 12B). Further, both AP and CP led to a decrease in proportion of T-bet expressing CD8⁺ (cytotoxic effector T cells) and CD4⁺ cells (Th1 cells) (FIGS. 12C, D). However, no differences in exhaustion markers, PD1 and TIM3 were observed on CD8⁺ T cells in AP and CP mice compared to controls (FIGS. 12E, F). Surprisingly, the inventors also observed a decrease in regulatory T cells (CD4⁺Foxp3⁺ cells) in the in WT mice with both AP and CP (FIG. 12G).

Taken together, these results indicate that pancreatitis increased the proportion of CD11c⁺ DCs in the pancreas of both iKPC* orthotopic and WT mice. Further, the inventors demonstrated that there is an increase in activated DCs and antigen presenting cDC1s. In addition, AP and CP in WT type mice generated a myeloproliferative and T cell suppression response, decreasing proliferating and granzyme B producing CD8⁺T cells. Further, AP and CP induced a decrease in frequency of T-bet expressing effector CTLs and Th1 cells indicating that the T cell suppression represents a host response to prevent autoimmune cytotoxic damage to the pancreas in mice with pancreatitis. In an environment of suppressed cell mediated cytotoxicity, further decrease in T regs with AP and CP suggests a peripheral tolerance mechanism and clonal depletion of global T cell populations in this context.

### Pancreatitis Recruits Activated Dendritic Cells to the Immune Microenvironment of KPC Mice

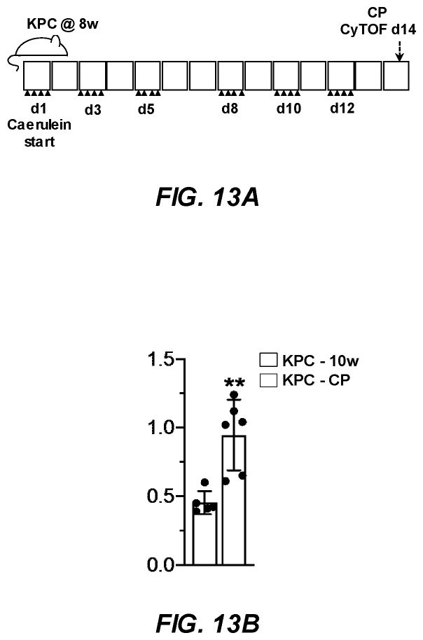

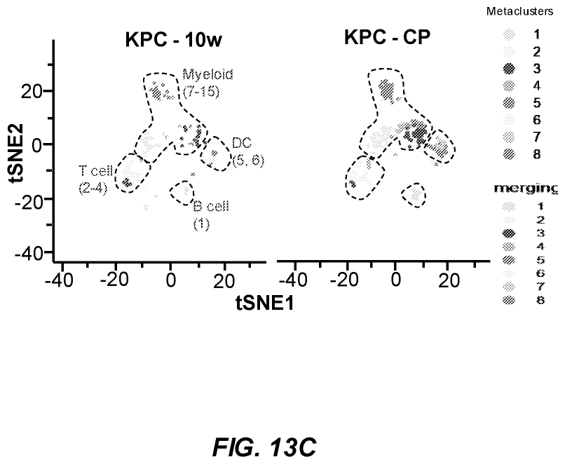

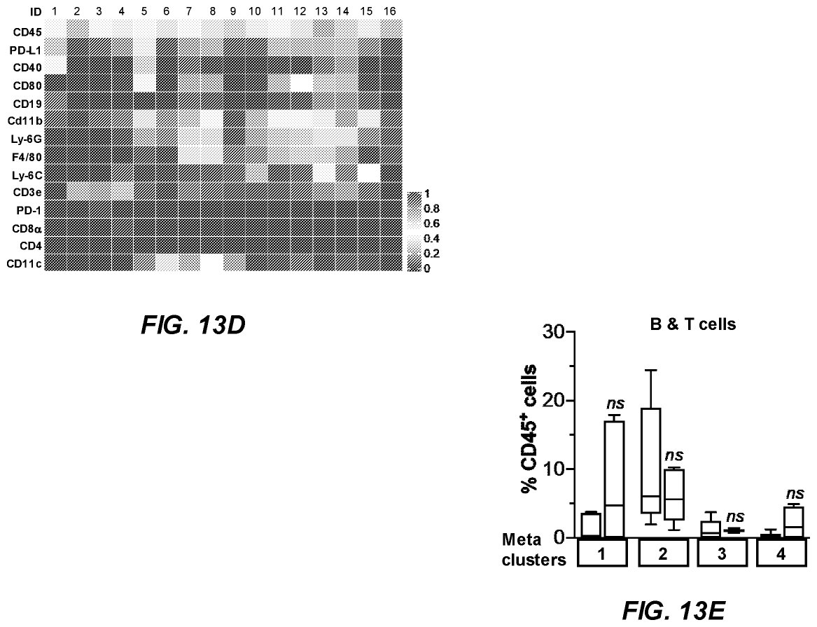

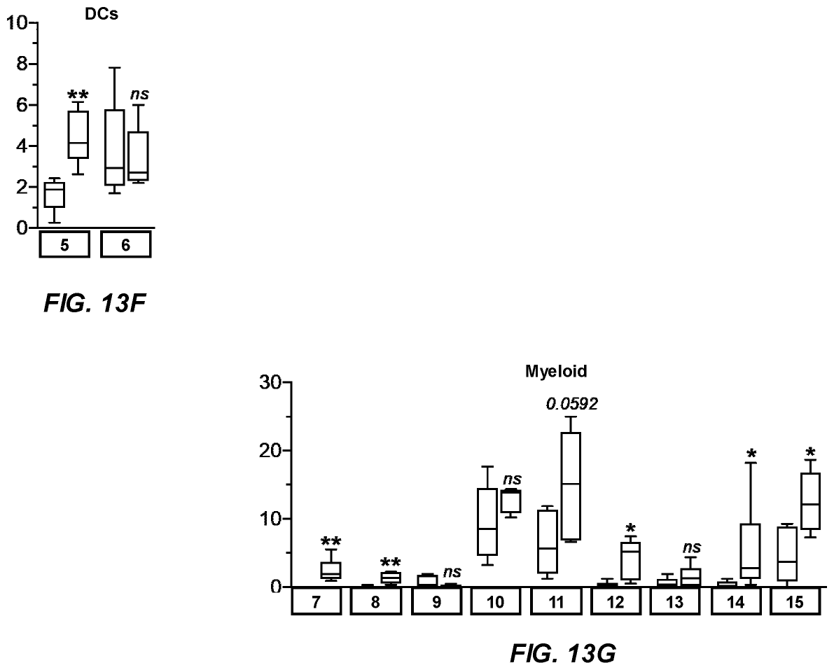

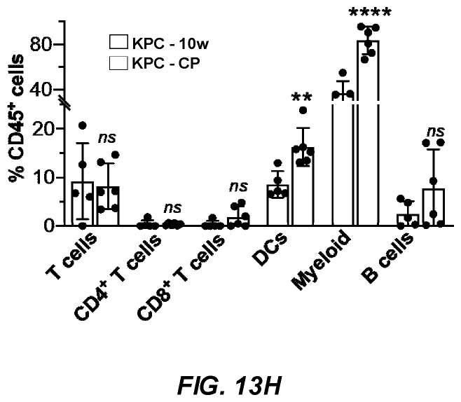

The inventors next probed whether the differences in pancreatic immune infiltrates between the WT and iKPC* orthotopic mice with pancreatitis were attributable to a baseline inflammation arising due to pancreatic injection of iKPC* cancer cells. To rule out the effect of baseline pancreatitis due to orthotopic tumor injection, the inventors analyze the pancreas infiltrating KPC (Pdx1-Cre; LSL-Kras^{G12D/+}; P53^{R172H/+}) with CP. The inventors induced CP in 8w old KPC mice and perform CyTOF analysis (similar to the iKPC* mice) to identify the immune infiltrates recruited by pancreatitis (FIG. 13A). The inventors used age matched KPC mice (˜10w) as controls for analysis of immune infiltrates. CP accelerated tumor growth in the KPC mice (FIG. 13B). FlowSOM analysis on CyTOF data with unsupervised hierarchical cluster identified 15 metaclusters (MCs) which were grouped into B cell (MC 1), T cell (MC 2-4), DC (MC 5, 6) and myeloid (MC 7-15) populations (FIGS. 13C-G). viSNE plots on these 15 MCs are shown in FIG. 13C. The KPC controls at 10 w had fewer CD45+ lymphocytes than their CP treated counterparts. Therefore, equal numbers of representative lymphocyte populations from the KPC—10w and KPC—CP mice pancreata are shown in the viSNE plot (FIG. 13C). Similar to the phenotype observed in the iKPC* mice, CP did not result in any changes in the B and T cell percentages (MC1-4) (FIGS. 13C-E, H). Among the DC MCs, a significant increase in the percentages of CD11c⁺ PDL1⁺ DCs (MC 5) was observed (FIGS. 13C, D, F). Among the myeloid MCs, a significant increase in the percentages of CD11c⁺ CD11b⁺ F4/80⁺ CD40⁺ CD80⁺ (MC7) and CD11c⁺ CD11b⁺ F4/80⁺ (MC8), CD11b⁺ PDL1⁺ CD80⁺ (MC12), CD11b⁺ PDL1⁺ CD80⁺ F4/80⁺ (MC14) and CD11b⁺ Ly-6G^{hi/+} Ly-6C^{low/-} (MC15) (FIGS. 13C-G). Comparison of the myeloid populations in the KPC Vs. iKPC* mice indicates that F4/80⁺ CD11c⁺ MCs (MC9, 10) (FIGS. 11C, E) in the iKPC* mice were a result of the inflammation associated with the orthotopic pancreatic injection. These F4/80⁺ CD11c⁺ populations did not increase further in the presence of CP due to caerulein injections (FIGS. 11C, E). Whereas in the autochthonous KPC mice, the F4/80⁺ CD11c⁺MCs (MC7, 8) (FIG. 13G) were nearly non-existent in the control mice and significantly increase in the presence of CP. In both the iKPC* and KPC mice with CP, CD11c⁺CD11b⁻F4/80⁻DC MCs viz. MC-7 in iKPC* (FIGS. 11C, E) and MC-5 in KPC (FIG. 13G) likely represent DCs involved in antigen presentation. In addition, CP also recruited CD11b⁺ F4/80⁺ macrophages (MC-11, 14) in the KPC mice (FIG. 13G). Therefore, caerulein induced CP and orthotopic injection resulted in the recruitment of inflammatory F4/80⁺ macrophages that also expressed CD11c. Further, caerulein induced CP recruited antigen presenting DCs that are negative for F4/80 in both the iKPC* and KPC mouse models.

Pancreatitis and DC Vaccine Sensitizes Orthotopic iKPC* Tumor Bearing Mice to Checkpoint Immunotherapy

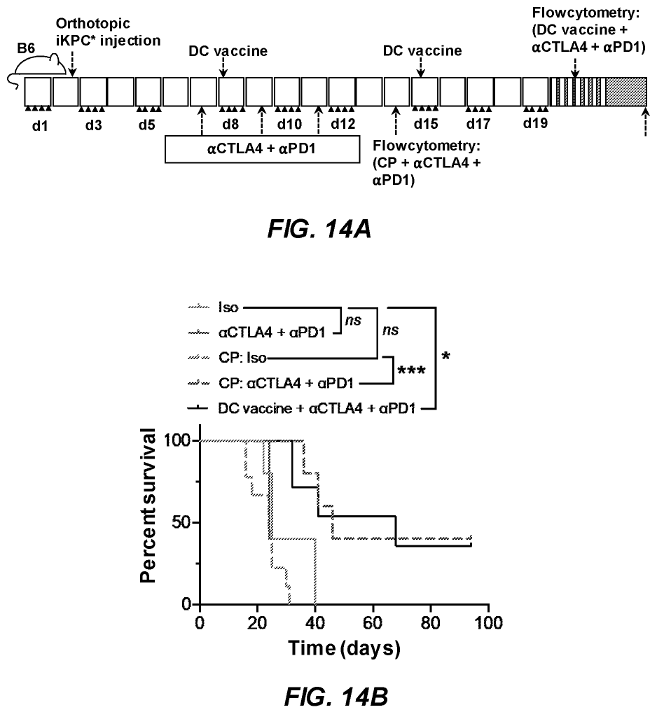

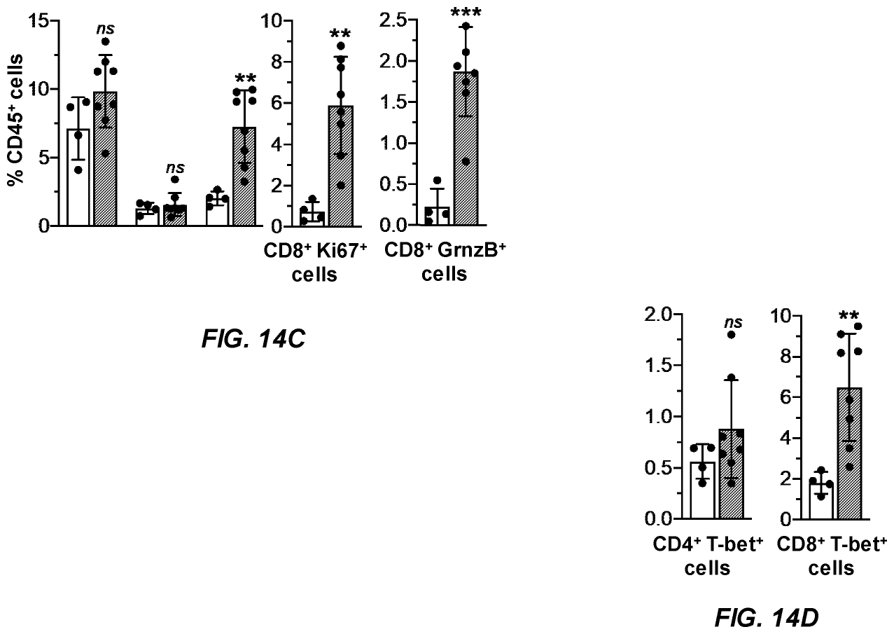

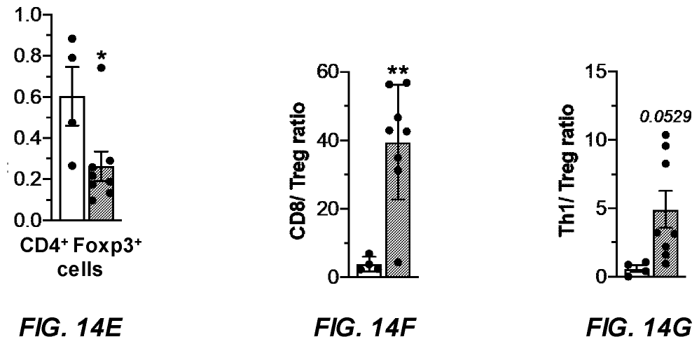

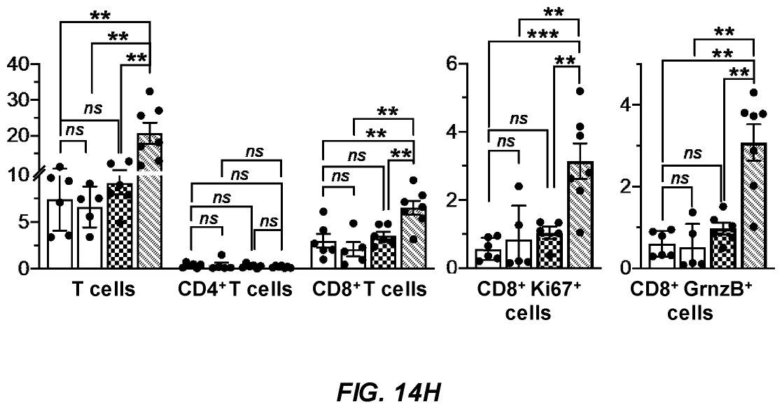

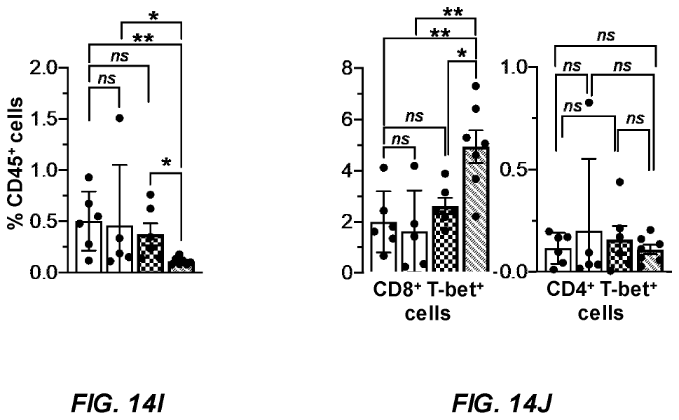

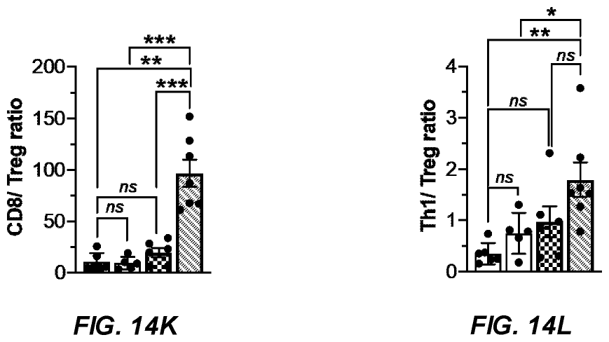

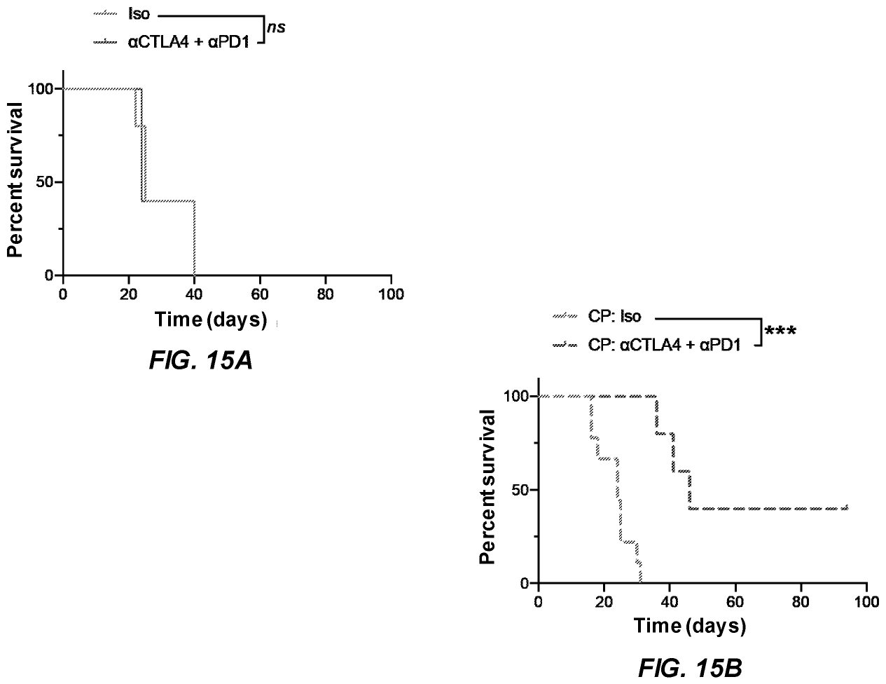

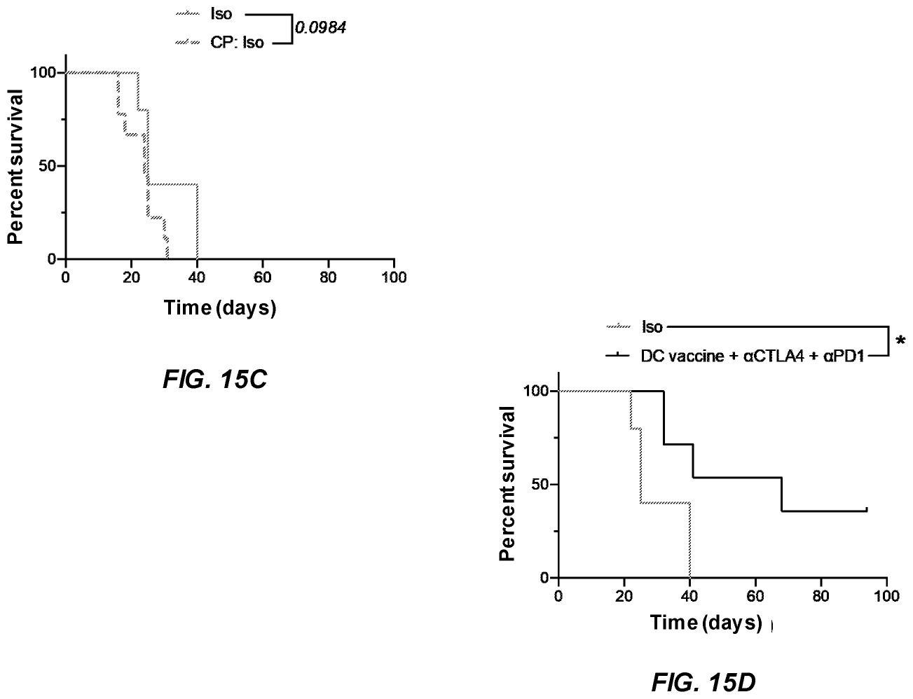

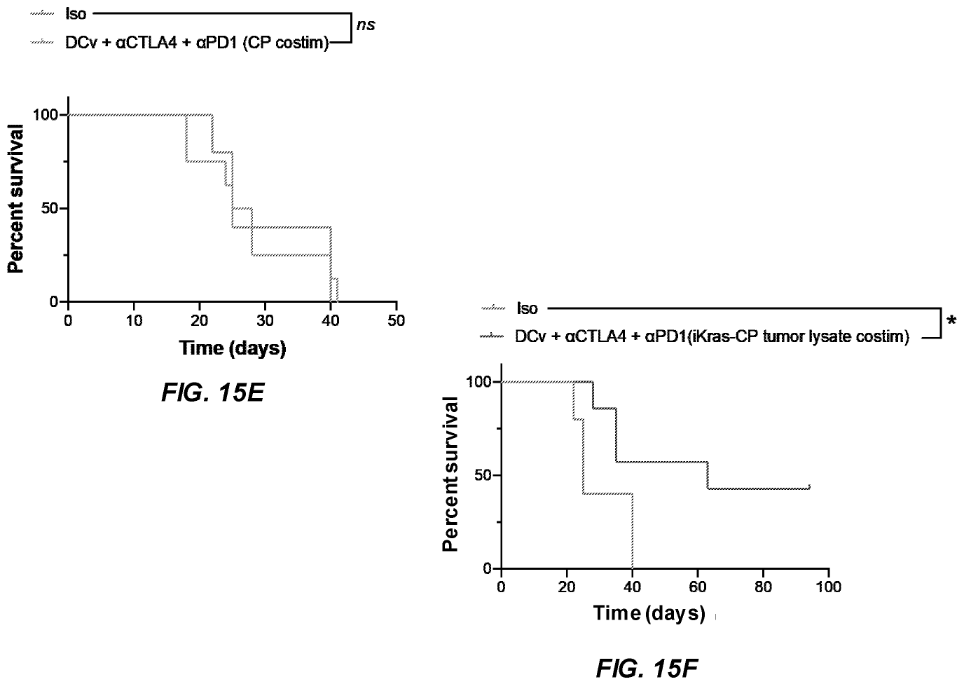

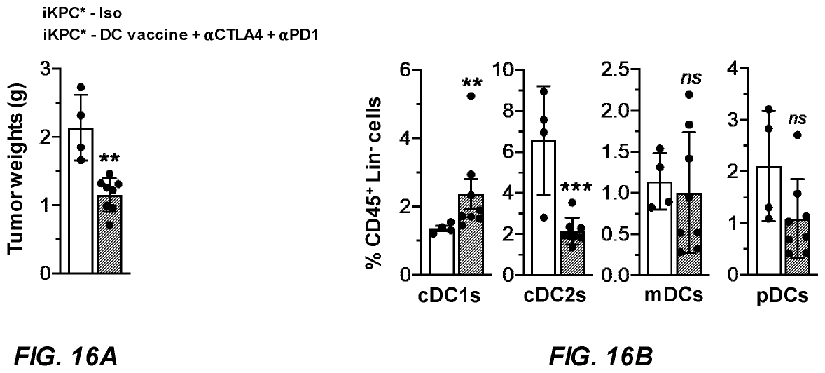

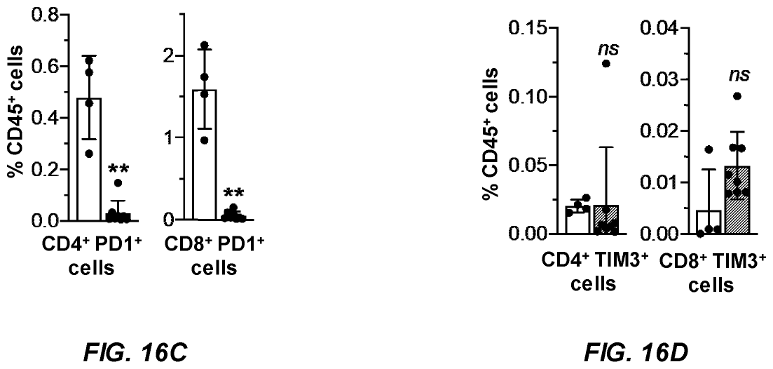

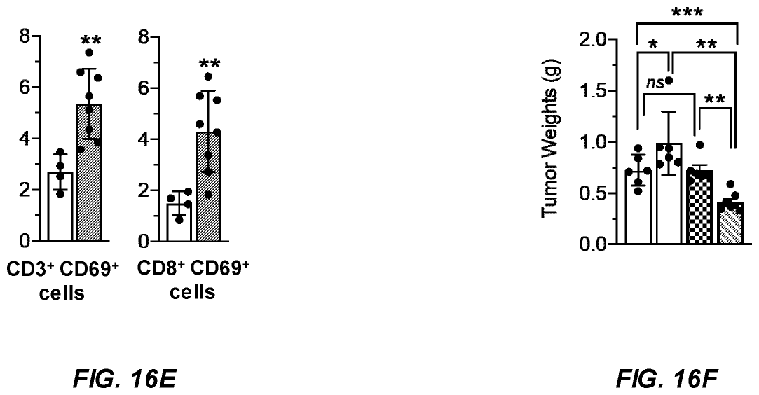

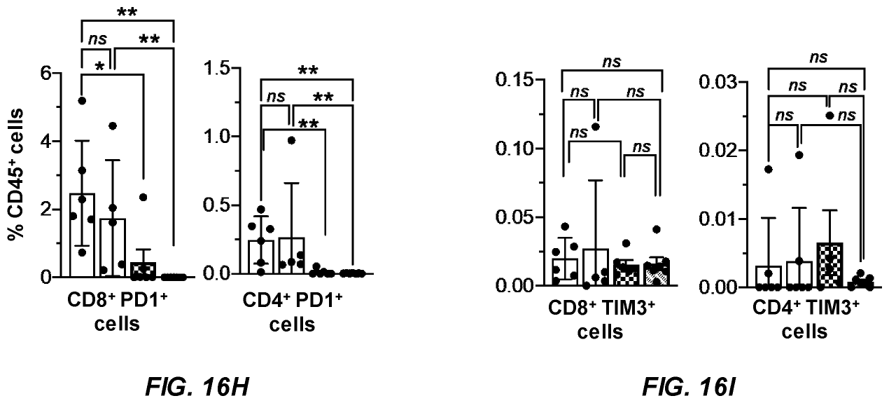

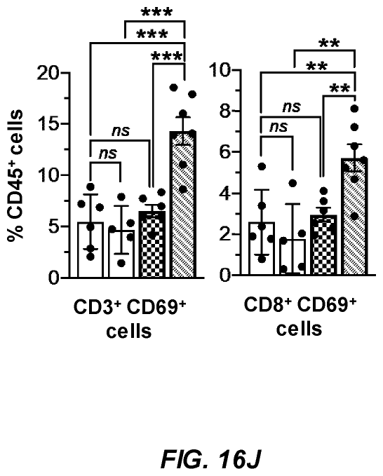

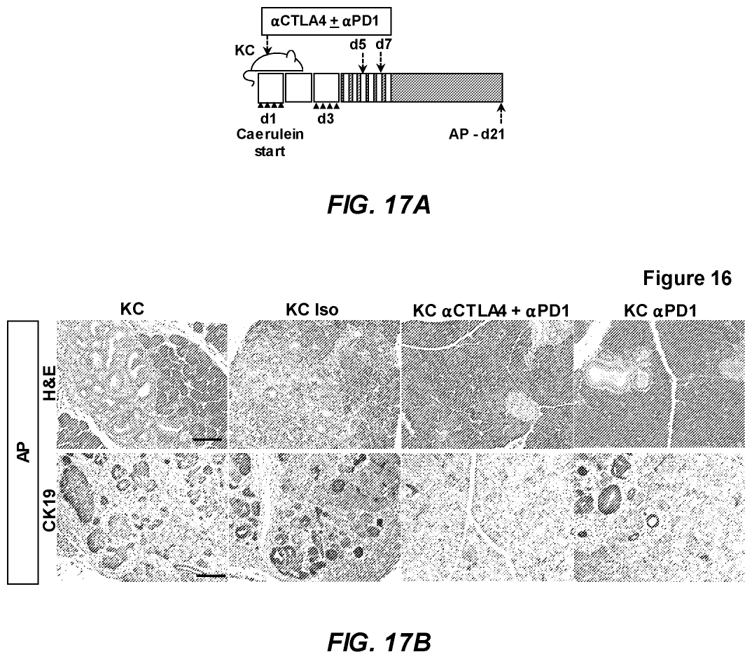

The inventors next determined if DC vaccines and CP in iKPC* tumors sensitize PDAC to checkpoint immunotherapy. Six to eight weeks old B6 mice were orthotopically injected with iKPC* cells and two cohorts of mice were treated with caerulein to induce chronic pancreatitis. iKPC* orthotopic tumor bearing mice with and without CP were treated with combination checkpoint immunotherapy (FIG. 14A). Another cohort of mice were treated with DC vaccine and combination checkpoint immunotherapy as indicated (FIG. 14A). The iKPC* orthotopic mice without CP did not respond to combination checkpoint immunotherapy, whereas CP sensitized these mice to immunotherapy (FIGS. 14B, 15A, B). CP did not significantly shorten the survival of the orthotopic tumor bearing iKPC* mice (FIGS. 14B, 15C). Further, treatment with DC vaccine and combination checkpoint immunotherapy resulted in tumor growth inhibition and enhanced survival in these mice (FIGS. 14B, 15D). A substantial portion (2 out of 5 mice) of the combination immunotherapy treated mice with DC vaccine and CP demonstrated cleared orthotopic iKPC* tumors. The inventors next analyzed immune infiltrates from DC vaccine and combination checkpoint immunotherapy treated mice after 2 doses of DC vaccine therapy on day21 following orthotopic injection (FIG. 14A). The DC vaccine+αCTLA4+αPD1 treated mice had lower tumor weights compared to the isotype treated iKPC* mice (FIG. 16A) with an increase in frequency of cDC1s and a concomitant decrease in cDC2s gated as a percentage of CD45⁺ Lin⁻ cells compared to the isotype mice (FIG. 16B). The DC vaccine+αCTLA4+αPD1 treated mice demonstrated a higher proportion of CD8⁺ T cells (FIG. 14C). Further analysis of T cells in these mice revealed an increase in proliferating, granzyme B and T-bet expressing CD8⁺ T cells, accompanied by a decrease in CD4⁺Foxp3⁺(Tregs) cells in the DC vaccine+αCTLA4+αPD1 treated mice (FIGS. 14C-E). Although no difference in the Th1 population was observed, the DC vaccine+αCTLA4+αPD1 treated mice showed an increase in the CD8/Treg ratio and increasing trend in the Th1/Treg ratio although this was not as robust as the CD8/Treg ratio (FIGS. 14F, G). Analysis of exhaustion markers revealed a decrease in PD1 expression in the CD4⁺ and CD8⁺ T cells, whereas no differences in TIM3 expression was observed in the DC vaccine+αCTLA4+αPD1 treated mice (FIGS. 16C, D). Analysis of CD69 marker which is associated with T cell memory and activation on T cells revealed an increase in frequencies of CD3⁺ CD69⁺ and CD8⁺ CD69⁺ T cells that are protective against tumor relapse (FIG. 16E).

Next, the inventors analyzed the immune infiltrates in the iKPC* tumors (at 2 weeks after iKPC* orthotopic injection) with CP and combination checkpoint immunotherapy. The iKPC* mice with CP had higher tumor weights compared to isotype treated iKPC* mice (FIG. 16F). Also, the iKPC* mice with CP treated with combination checkpoint immunotherapy had smaller tumors compared to isotype and checkpoint treated iKPC* mice (FIG. 16F). Analysis of immune infiltrates revealed an increase in the proportion of CD3⁺ and CD8⁺ T cells in the tumor microenvironment of iKPC* mice with CP treated with combination checkpoint immunotherapy (FIG. 14H). Further analysis of T cells in these mice revealed an increase in proliferating, granzyme B and T-bet expressing CD8⁺ T cells, accompanied by a decrease in CD4⁺ Foxp3⁺ (Tregs) cells in CP mice treated with αCTLA4+αPD1 (FIGS. 14H-J). Also, the CD8⁺ T cells demonstrated a decrease in PD1 activity in both the checkpoint immunotherapy groups with and without CP, whereas no difference in TIM3 expressing CD4⁺ and CD8⁺ T cells were observed between the groups (FIGS. 16G, H). No differences in Th1 cell population were observed between any of the groups (FIG. 14J). However, an increase in CD8/Treg ratio and an increase in the Th1/Treg ratio were observed in CP mice treated with αCTLA4+αPD1 (FIGS. 14K, L). The CP mice treated with combination checkpoint immunotherapy also demonstrated an increase in CD69 expressing CD3+ and CD8+ T cells (FIG. 16I). Collectively, the inventors' results indicate that PDAC tumors are resistant to checkpoint immunotherapy due to the lack of antigen presenting DCs in the TME. DC vaccine therapy and chronic pancreatitis recruited antigen presenting DCs that render PDACs amenable to combination checkpoint immunotherapy.

### Pancreatitis Sensitizes PanIN Lesions to Checkpoint Blockade

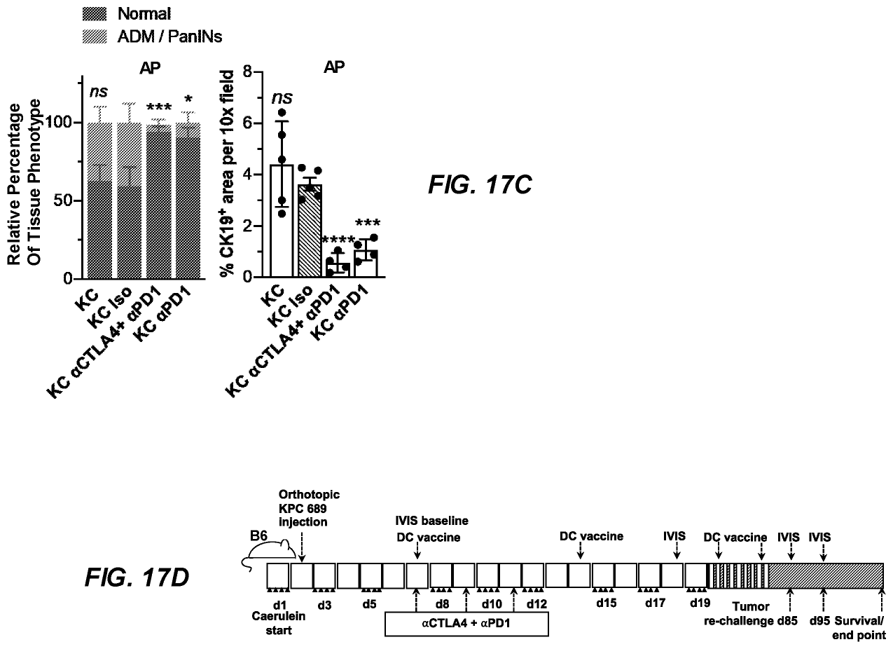

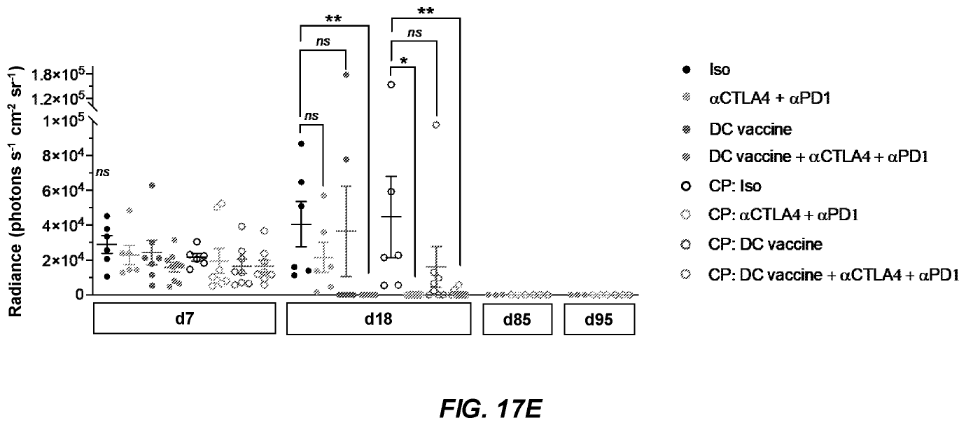

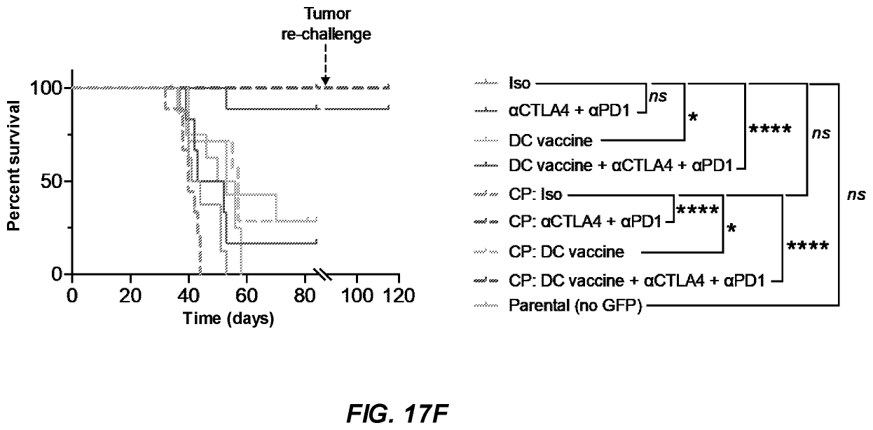

Next, the inventors probe whether presence of underlying pancreatitis renders the PanIN lesions sensitive to checkpoint blockade. Multiple studies in patients and in pre-clinical models have established that checkpoint immunotherapy has failed to produce durable survival responses in PDAC(11-13). The inventors investigate whether combination of αCTLA4+αPD1 or αPD1 monotherapy would inhibit tumor initiation in the presence of antigen presenting dendritic cells recruited during AP (FIG. 17A). When the KC mice were sacrificed 3 weeks later for analysis of checkpoint blockade response, the inventors observed that there was a decrease in PanINs in the αCTLA4+αPD1 and αPD1 treated KC mice with pancreatitis compared to the Isotype and untreated KC mice with AP (FIGS. 17B, C). Therefore, the inventors' results indicated that the presence of underlying pancreatitis sensitized the KC mice to checkpoint blockade.

### Pancreatitis and DC Vaccine Therapy Sensitizes Orthotopic KPC Tumors to Checkpoint Blockade

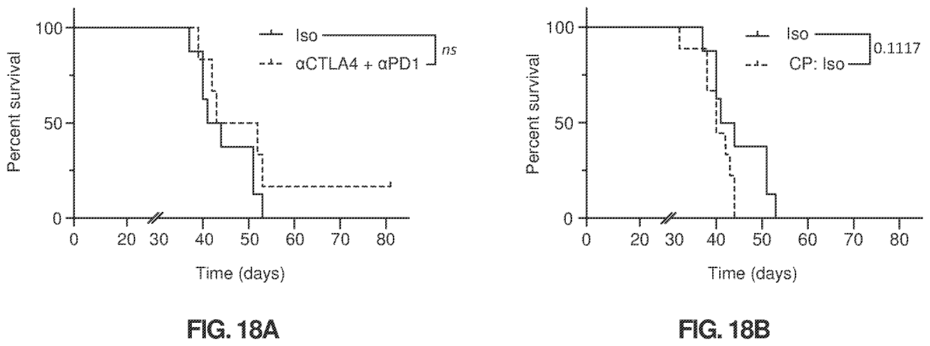

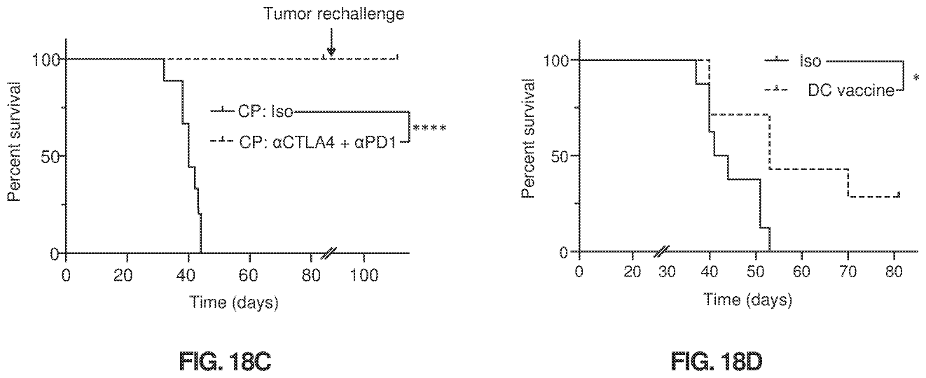

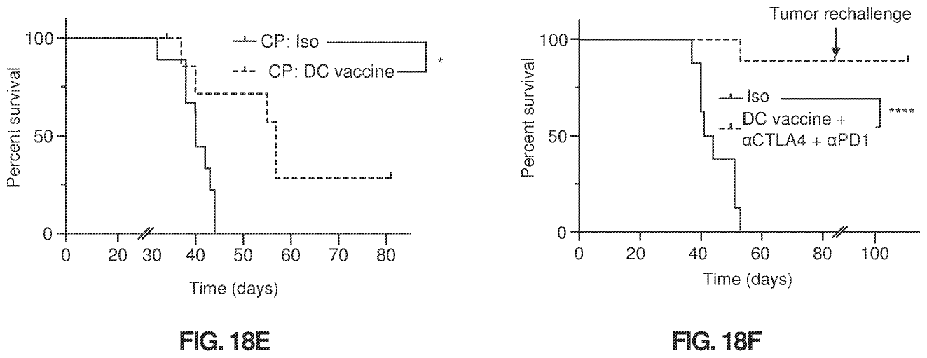

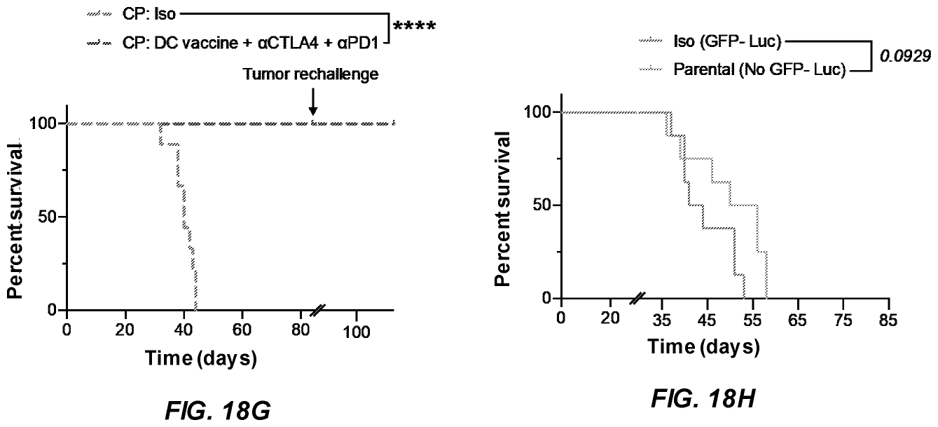

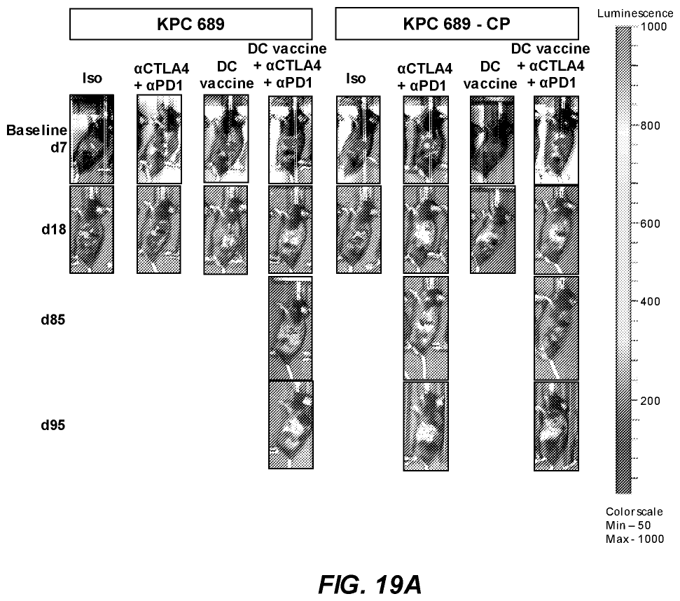

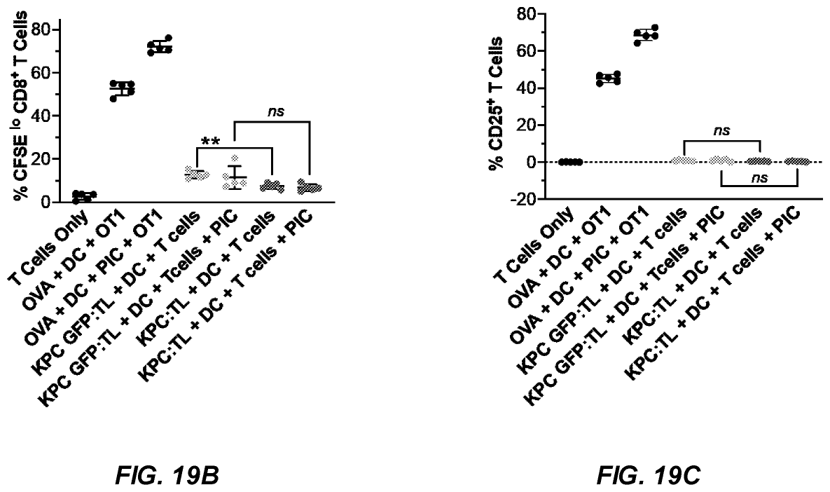

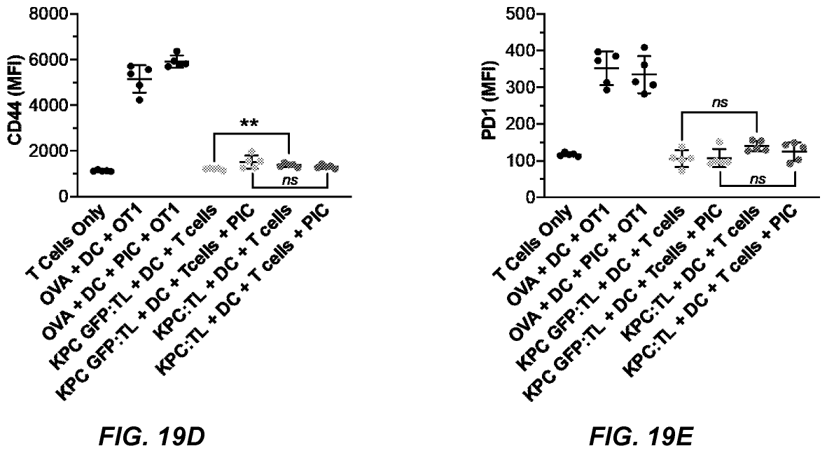

Next, the inventors validate the inventors' findings from the iKPC* model in a second orthotopic tumor model. The inventors utilize an orthotopic KPC 689 tumor model to determine the impact of checkpoint immunotherapy in combination with DC vaccines and its role in restricting established PDAC with underlying pancreatitis (FIG. 17D). Six to eight weeks old B6 mice were orthotopically injected with 5×10⁵ bioluminescent KPC 689 GFP-Luc cells (Pdx1-Cre; LSL-Kras^{G12D/+}; P53^{R172H/+} cells transfected with GFP-Luc). One cohort of mice were simultaneously treated with caerulein to induce CP for 3 weeks as described earlier, whereas the other cohort of mice were allowed to develop tumors in the absence of CP. Following baseline IVIS imaging after one week, tumor bearing mice were treated with DC vaccine and checkpoint immunotherapy (FIG. 17D). In the absence of CP, combination checkpoint immunotherapy (αCTLA4+αPD1) did not result in tumor growth inhibition or significant improvement in survival compared to isotype treated mice (FIGS. 17D-F, 18A). Although presence of CP accelerated tumor initiation in the KC mice, chronic pancreatitis did not result in statistically significant shortening of survival in the KPC 689 tumor bearing mice (FIGS. 17G, 18B). In the presence of chronic pancreatitis, combination checkpoint immunotherapy led to clearance of KPC689 tumors (FIGS. 17E, F, 18C). Administration of DC vaccine also improved the survival in mice with and without CP (FIGS. 17E, F, 18D, E). Administration of combination checkpoint immunotherapy in DC vaccine treated mice enhanced clearance of KPC 689 tumors in mice with and without CP (FIGS. 17E,F, 18 F, G). Collectively, these results indicated that activated dendritic cells recruited to the pancreatic TME by either DC vaccine therapy or chronic pancreatitis sensitize the tumors to checkpoint immunotherapy. Further, to determine T cell memory in the 3 mice cohorts that cleared tumors, viz. DC vaccine+αCTLA4+αPD1, CP+DC vaccine+αCTLA4+αPD1, CP+αCTLA4+αPD1, the inventors re-challenged these mice with orthotopic injection of KPC 689 cells on day 85 (FIG. 17E). IVIS imaging 10 days after re-challenge did not reveal the presence of tumors in any of the groups (FIG. 17E). Representative IVIS images of all treatment cohorts are shown in FIG. 19A.

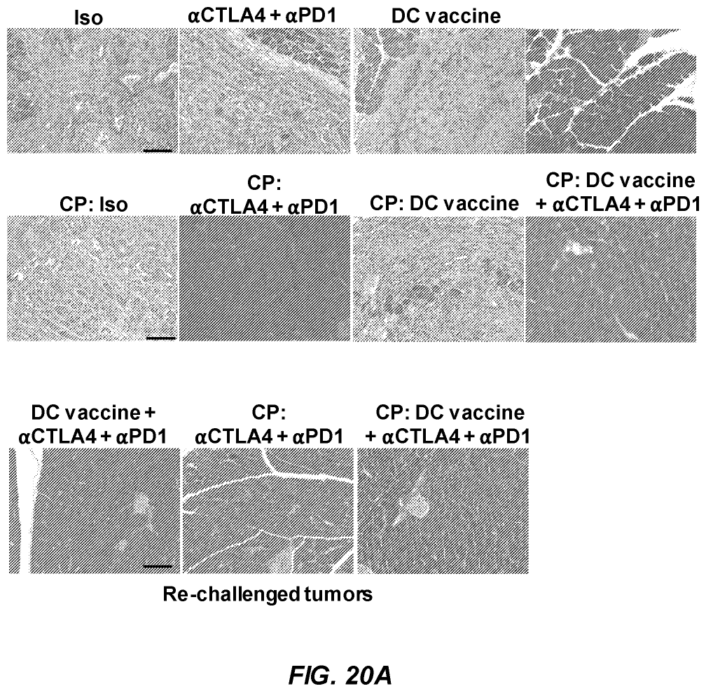

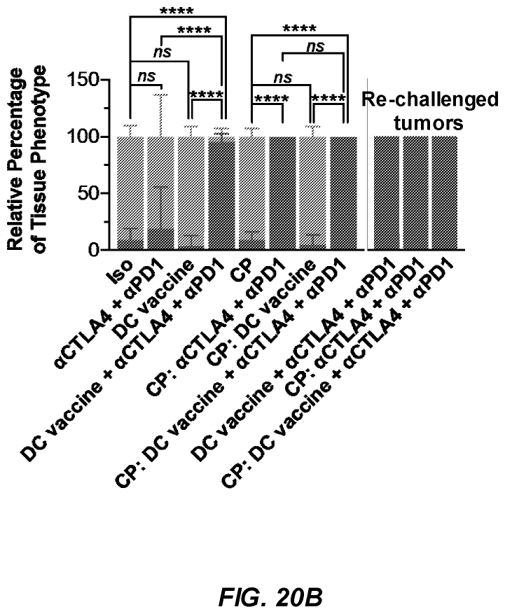

Next, the inventors probed if the GFP expressed by the KPC689 cells enhanced immunogenicity on the pancreatic cancer cells leading to tumor clearance in response to DC vaccine and checkpoint immunotherapy. Studies that utilize tumor cell lines expressing GFP in murine models have indicated enhanced anti-tumor immune response compared to their parental tumor lines (27, 28). To assess the impact of GFP in eliciting an anti-tumor immune response in the inventors' model, the inventors compared the baseline survival of parental vs. GFP-Luc expressing KPC 689 tumor bearing mice. No significant difference in survival was observed between the parental and GFP-Luc expressing tumor bearing mice (FIG. 17E) indicating that expression of GFP-Luc did not result in an immunoediting response. Further, the inventors evaluated CD8⁺ T cell activation in-vitro when co-cultured with stimulated CD103⁺ cDC1 cells in tumor lysates with and without GFP. The inventors used ovalbumin specific CD8⁺ T cells (OT-1 cells) as a positive control for CD8 activation. After four days of co-culture of cDC1s and CD8⁺ T cells with parental or GFP-Luc expressing KPC689 tumor cell lysate, the inventors analyzed CD8⁺ T cell activity and exhaustion including % CFSE^{lo}, CD25, CD44 and PD1 expression on CD8⁺ T cells. The inventors observed slight differences between groups with and without TLR agonist poly (I:C), however changes were marginal compared to OT-1 conditions (FIGS. 19B-D). Thus, both KPC689 tumor cell lysates with and without GFP Luc demonstrated similar levels of CD8⁺T cell activation, indicating that GFP does not likely contribute to enhanced tumor clearance. The inventors next analyzed tumor histology of DC vaccine, chronic pancreatitis and checkpoint immunotherapy treated mice at end point. The inventors' data indicated that CP mice with αCTLA4+αPD1, DC vaccine+αCTLA4+αPD1 and CP mice with DC vaccine+&CTLA4+αPD1 treatment cleared tumors and showed completely normal pancreas histology even following tumor re-challenge in a subset of these mice (FIGS. 20A, B).

Tumor Infiltrating Dendritic Cells Correlate with CD8⁺ T Cell Infiltration and Better Prognosis in Human PDAC

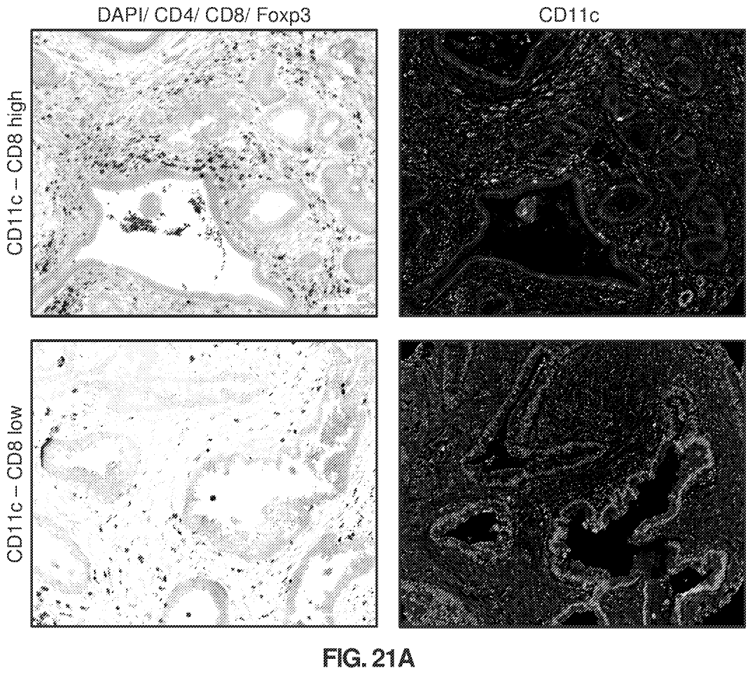

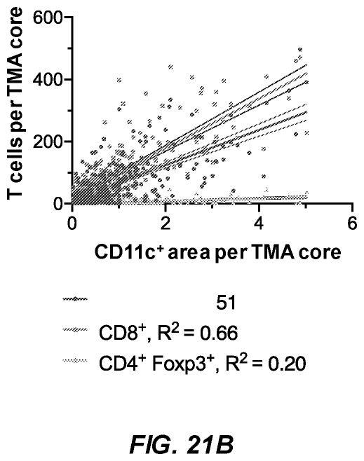

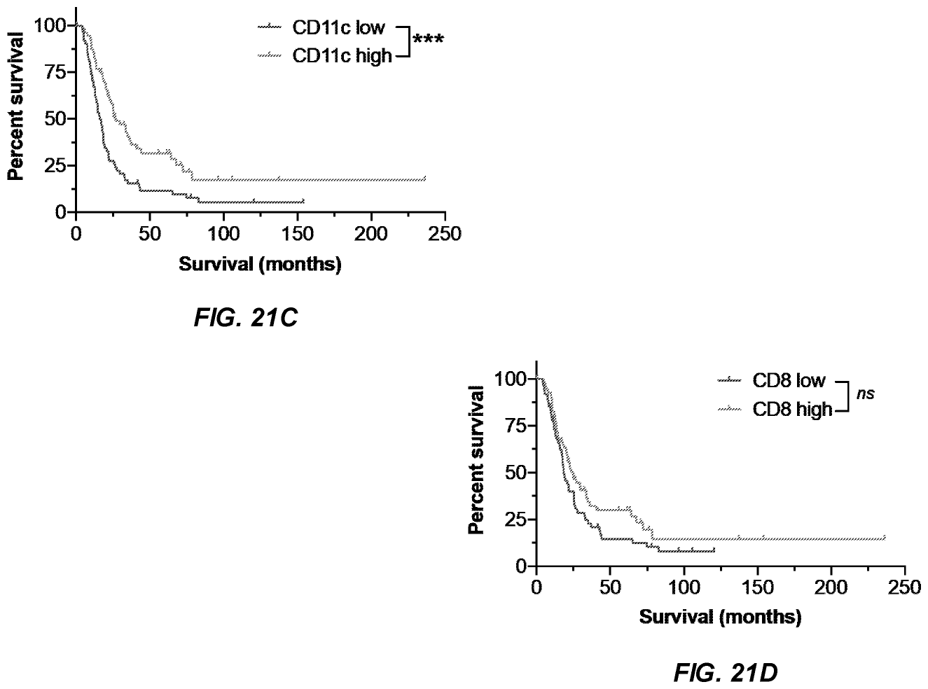

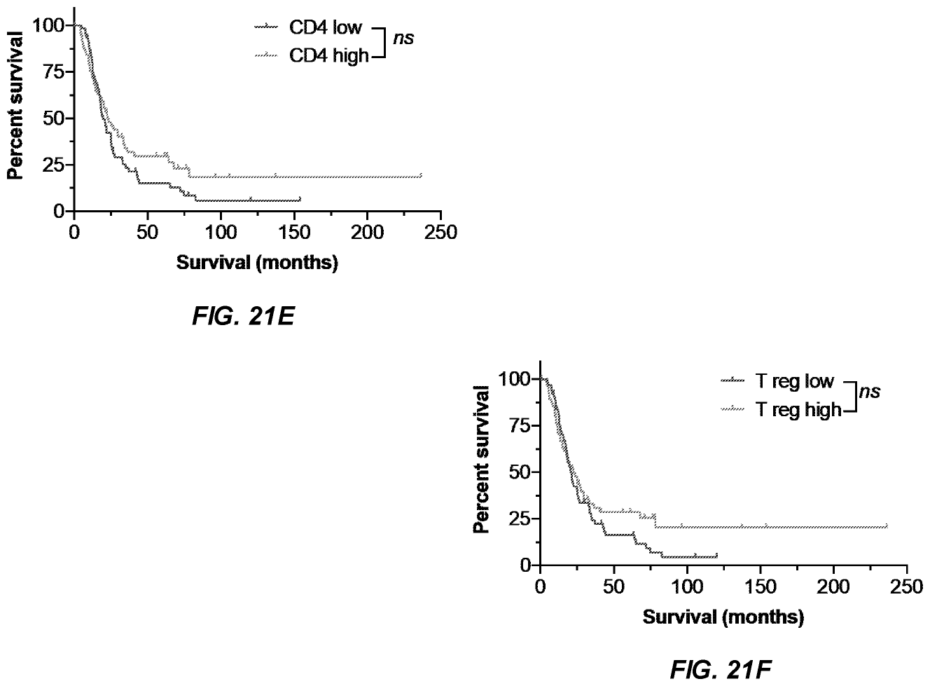

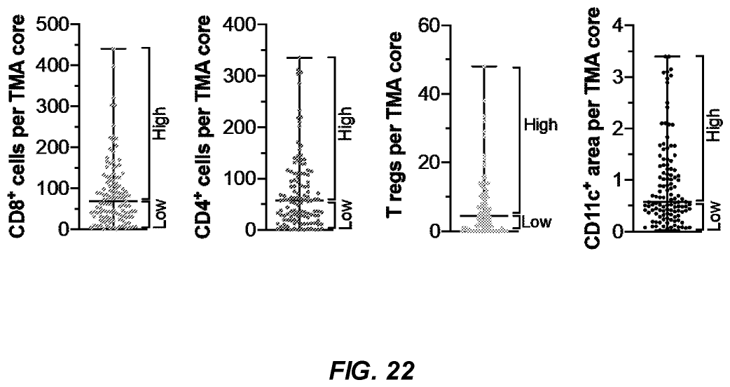

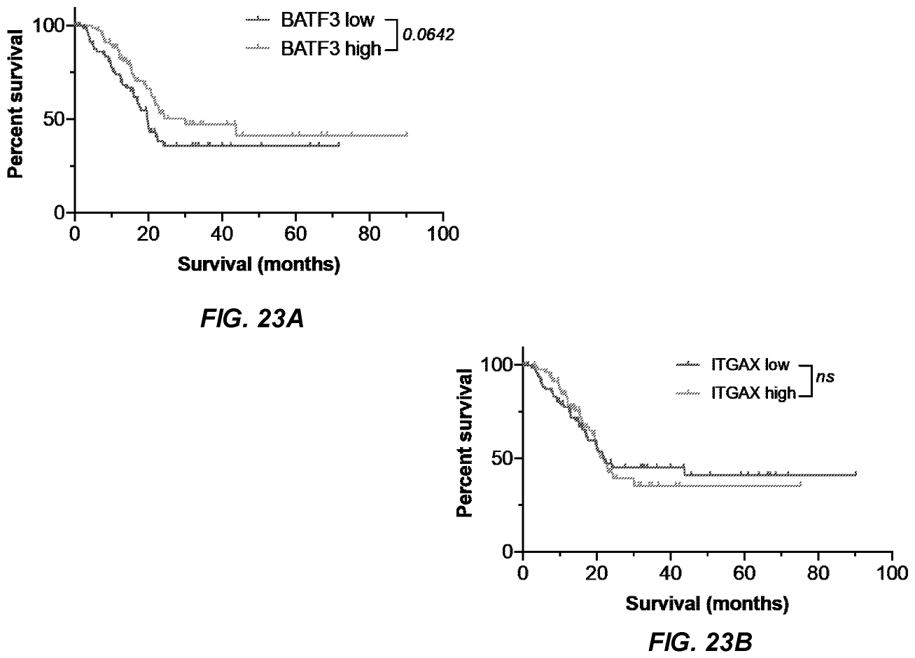

Based on the impact of DCs in modulating T cell response and survival in murine PDAC, the inventors next assessed the contribution of DCs in the setting of human PDAC. The inventors performed immune-staining for CD4, CD8, Foxp3 and CD11c markers in treatment naïve human PDAC tumor microarray (TMA) samples. Analysis of CD4⁺, CD8⁺, Tregs and CD11c⁺ cells in these PDAC TMAs demonstrated a good correlation (R²=0.66) between tumor infiltrating CD8⁺ T cells and CD11c⁺ dendritic cells (FIGS. 21A, B). Infiltration of DCs had a weaker correlation with CD4⁺ T cells (R²=0.51) and a poor correlation with Treg infiltration (R²=0.2) in human PDAC (FIGS. 21A, B). The inventors next divided tumors as ‘high’ and ‘low’ for each cell type based on the median number of tumor-infiltrating cells to analyze disease specific survival in these patients (FIG. 22). CD11c high patients had a significantly longer disease specific survival (DSS) compared to CD11c low PDAC patients (FIG. 21C). Although there was a good correlation between tumor infiltrating CD11c⁺ DCs and CD8⁺ T cells, CD8⁺ T cells did not predict survival of PDAC patients (FIG. 21D). Further, CD4⁺ T cells and Tregs infiltration did not predict survival in human PDAC tumors (FIG. 21E, F). Next, the inventors analyzed the DC infiltration in 173 treatment naïve patients from the TCGA-Pancan dataset that performed bulk RNA sequencing on human PDAC tumors. Although ITGAX RNA expression (marker for CD11c⁺ cells) in the TCGA dataset did not stratify survival in the TCGA dataset (FIGS. 23A-B), the inventors identified higher BATF3 expression, a marker specific for cDC1s in humans predicted better prognosis in PDAC patients (FIG. 22A). The human PDAC analysis revealed that DCs play an important role in altering the course of PDAC and the lack of tumor-infiltrating DCs could contribute to refractoriness of PDAC to T cell targeting therapies.

In conclusion, these studies provide a thorough, context dependent analysis of T cell function and its interaction with DCs in the PDAC TME. The presence of antigen presenting DCs recruited either by pancreatitis or by exogeneous administration of CD103⁺ cDC1 vaccine, sensitizes the PDAC TME to checkpoint immunotherapy. These studies indicate that patients with a history of chronic pancreatitis could benefit from immunotherapy and provides rationale for combining DC vaccines with checkpoint immunotherapy or CD4⁺ T cell targeted therapies in PDAC.

### Material and Methods

Animal studies: The genotyping and tumor progression of the Pdx1-cre; LSL-Kras^{G12D/+}; P53^{R172h/+} (KPC) and Pdx1-cre; LSL-Kras^{G12D/+} (KC) mice have been described previously(31). The inventors crossed KPC to CD4^{-/-} (Cd4^{tm1Mak})(32) or CD8^{-/-} (Cd8^{tm1Mak})(33) mice (both kindly provided by Dr. Tak Mak, University Health Network—University of Toronto) to obtain KC, KC CD4^{-/-}, KC CD8^{-/-}, KPC, KPC CD4^{-/-} and KPC CD8^{-/-} mice. For orthotopic experiments, 6-8w old B6 mice were injected with 5×10⁵ primary PDAC cell lines viz. KPC689 GFP Luc cell line (34) and iKPC* cell line (35) (kindly provided by Dr. Haoqiang Ying) in the pancreas. The iKPC* cell line has a tetracycline inducible tetO-LSL Kras^{G12D/+} allele and was maintained on doxycycline (Dox) water (Dox 2 g/L, sucrose 20 g/L) starting simultaneously with orthotopic injection throughout the experiment. For the tumor re-challenge study, 5×10⁵ KPC689 GFP Luc cells were injected into the pancreas at day 85 following the initial injection. Tumor radiance (photons s⁻¹ cm⁻² sr⁻¹) was monitored for the KPC 689 GFP Luc cell line injection using IVIS imaging (Xenogen spectrum) under uniform conditions across all experimental groups. Mice were injected with luciferin (100 mg/kg, at 10 mg/ml concentration) intraperitoneally and imaged under isoflurane anesthesia 10 minutes following injection. For pancreatitis induction, cerulein was injected at a final volume of 100 μL (dose −50 μg/kg per mouse), four times a day (six hourly injections), on alternate days for acute pancreatitis and three times a week for 3 weeks to induce chronic pancreatitis as described earlier(19). For experiments to neutralize CD11c⁺ DCs, 500 μg of anti-mouse CD11c (ThermoFisher scientific, N418) (1 mg/mL) was injected intraperitoneally on days 0, 5, 10 and 15 for each mouse. For checkpoint immunotherapy, anti-mouse CTLA4 (BioXcell, 9H10, BE0131) and/or anti-mousePD-1 (BioXcell, 29F.1A12, BE0273) were injected three times intra-peritoneally as indicated, starting dose 200 μg followed by 2 doses of 100 μg each in final volumes of 200 μL and 100 μL PBS respectively. Control mice were treated with respective isotype antibodies in the same route, time and dosing as the neutralizing antibodies as recommended by the manufacturer. For DC vaccine, 1.5-2×10⁶ cDC1s were injected intraperitoneally every week for 4 weeks in the KPC 689 GFP Luc and for 2 weeks in the iKPC* orthotopic tumor bearing mice.

DC vaccine: Preparation of the CD103⁺cDC1 vaccine has been described (30, 36). Briefly, B6 mouse (6-10 w old) bone marrow culture was established following RBC lysis at a concentration of 1.5×10⁵ cells/mL in cRPM1 (10% heat-inactivated fetal bovine serum (FBS) (Atlanta Biologicals, Atlanta, Georgia, USA), 1% penicillin-streptomycin, 1 mM sodium pyruvate, and 50 μM B-mercaptocthanol) supplemented with 50 ng/ml hFIt3-L (PeproTech, 10773-618) and 2 ng/mL GM-CSF (PeproTech, 315-03). The culture is supplemented on day 5 with 5 mL of cRPM1 and subsequently, the non-adherent cells are re-plated at a concentration of 3×10⁵ cells/mL supplemented with the same amount of hFIt3-L and GM-CSF on day 9. The supernatant with non-adherent cells were collected on day 15-17 for co-stimulation with tumor lysate. The cell pellet is stained for the following markers: CD11c, B220, CD24, CD172a and CD103. The cDC1s (L/D⁻ CD11c⁺ CD24⁺ CD172⁻CD103⁺ B220⁻) are sorted on FACS aria fusion sorter and plated at 2-4×10⁶ cells/mL in cRPM1, co-stimulated with tumor lysate prepared from either KPC 689 GFP Luc or IKPC* cells (Lysate: cDC1 ratio=2:1). The culture is supplemented with 20 μg/mL Poly I:C (Sigma-Aldrich, P4929) and 2 ng/mL GM-CSF for 4 hours. Subsequently, 1.5-2×10⁶ cDC1s were resuspended in 100 μL PBS and injected intraperitoneally as indicated.

For in-vitro CD8⁺ T cell stimulation experiments to compare KPC689 GFP Luc tumor lysate vs. KPC parental tumor lysates, the inventors stimulate cDC1s with poly I:C, the respective tumor lysates (Ratio of cDC1s: tumor cell lysate=2:1) or with both. The inventors plate the T cells and cDC1s at a concentration of 1×10⁵ cells/mL, 100 μL of each in a 96 well plate. After incubation, the cells are spun down and washed in FACS buffer. Then, the cells are stained with a cocktail of antibodies for CD3, CD8, PD1, CD25 and CFSE for 30 minutes on ice. Cells are fixed in 1.6% formaldehyde and analyzed by flowcytometry. All antibodies were used at a concentration of 1:200 for cDC1 sorting and CD8+ T cell stimulation experiments.

Immunostaining: For single stained immunohistochemistry (IHC), 5 μm thick formalin fixed paraffin embedded (FFPE) slides were deparaffinized and antigen retrieval was performed in indicated buffers at 95° C. for 20 minutes. For CK19, CD11b, CD68 and CD11c staining, citrate buffer (pH=6) was used, whereas for CD4, CD8 and Ki67 staining. Tris-EDTA buffer (pH=9) was used for antigen retrieval. Subsequently, slides were blocked in 1.5% bovine serum albumin in PBST (0.1% Tween 20) for 30 minutes. Slides were then incubated in 3% H₂O₂ in PBS for 15 minutes. Primary antibodies CK19(Abcam, Ab52625, 1:250), CD11b (Abcam, Ab13357; 1:500), anti-mouse CD11c (Cell signaling technology, CST 97585S, 1:350), anti-human CD11c (Abcam, Ab52632, 1:100), CD4 (Abcam, Ab183685, 1:400), CD8 (Cell signaling technology, 98941s, 1:250) and Ki67 (ThermoScientific, RM-9106-S, 1:100) were diluted in 1.5% BSA in PBST and incubated overnight at 4° C. For all IHC, sections were incubated with biotinylated secondary antibody for 30 minutes followed by ABC kit (VECTASTATIN, ABC kit, Standard, PK-6100) for 30 minutes. Next, DAB and counterstaining with hematoxylin were performed and DAB positivity was quantified by examining multiple random visual fields. For CD68 (M0814, Dako, 1:200) immunostaining, Mouse-on-Mouse (MOM) kit (Vector Laboratories) following the manufacturer's instructions. For the thymus and spleens of KPC, KPC CD4^{-/-} and KPC CD8^{-/-} mice, 5 μm-thick cryostat OCT sections were fixed in acetone at 4ºC for 5 min, blocked in 1.5% BSA in PBS for 30 min, stained with primary antibodies—CD4 (Abcam, Ab183685, 1:400) or CD8 (Abdserotec, MCA1767T, 1:100) in 1.5% BSA in PBS (1 h at RT) and secondary antibodies (Goat anti-rabbit (H+L), Alexa Fluor Plus 488, ThermoFischer, A32731, 1:250 for CD4 primary or Goat anti-rat IgG (H+L), Alexa Fluor 400, 1:250 for CD8 primary) (30 min at RT).

Immunofluorescence staining performed using Tyramide signaling amplification (TSA) has been described elsewhere (37).

Flow cytometry: Tumors or pancreas were minced and digested in 5 mL of Collagenase P. 1.5 mg/mL (Sigma-Aldrich) in HBSS at 37ºC for 20 minutes. Subsequently, multiple washes were performed in cRPMI and filtered using 70 μm strainer (Corning 352350) and spun down. Cells were washed and resuspended in FACS buffer. Subsequently, cells were incubated in RBC lysis buffer (Thermofisher, 00-4300) for 5 minutes. Cells were stained with 100 μL surface antibody cocktail diluted in FACS buffer, 20% brilliant stain buffer (BD Bioscience, 566349), Live/dead stain (eBioscience, 65-0865-14) and 50 μg/mL anti-mouse CD16/32 (TONBO biosciences, 40-0161) for 30 minutes on ice, protected from light. For intracellular staining, cells were fixed and permcabilized in Foxp3/Transcription Factor Staining Buffer Set (cBioscience, 00-5523-00) and incubated with intracellular antibodies diluted in Fixation/Permeabilization diluent (eBioscience, 00-5223) for 30 minutes. Subsequently, cells were fixed with fixation buffer (BD Bioscience 554655) and data were acquired using Fortessa-X20 and analyzed with FlowJo v10.

Mass cytometry: Tumors or pancreas were minced and digested as described earlier in the flow cytometry section. Following RBC lysis, CD45⁺ lymphocytes were flow sorted and 1×10⁶ cells were used for staining with antibody cocktail (Table 4) with anti-mouse CD16/32 (TONBO biosciences, 40-0161) for 30 minutes at room temperature in a final volume of 100 μL in maxpar cell staining buffer (Fluidigm, 201068). Cisplatin (Fluidigm, 201064) viability staining was added at a 5 μM final concentration in maxpar PBS (Fluidigm, 201058). The cells were fixed in 1.6% formaldehyde solution diluted from the 16% formaldehyde stock ampule (Thermofisher, 28906) in maxpar PBS for 10 minutes in room temperature. Cells were then incubated in Cell-ID Intercalator Ir (Fluidigm, 201192A) prepared in Maxpar Fix and Perm Buffer to a final concentration of 125 nM overnight at 4º C. The cells were resuspended in maxpar water (Fluidigm, 201069) and analyzed in Fluidigm Helios Mass Cytometer. Mass cytometry data was initially processed and manually gated in Flowjo (version 10.7.1). Live CD45⁺ cells of each sample with the same percentage were exported and utilized for the downstream clustering analysis. The inventors conducted the downstream analysis using the approaches described in R (version 4.0.2) package CyTOF workflow (version 1.7.2). Specifically, R package FlowSOM (version 1.20.0) was employed to computationally define the initial cell clusters using the following parameters: CD45, PD-L1, CD40, CD80, CD19, CD11b, Ly-6G, F4/80, Ly-6C, CD3c, PD-1, CD8a, CD4 and CD11c, following by identification cell metaclusters based on the heat map. Dimensionality reduction analysis was conducted by t-stochastic neighbor embedding (t-SNE) with R package scatter (version 1.16.2).

Statistical analysis: Statistical tests were performed using GraphPad Prism 8 and R-studio. To assess the normality of distribution, Shapiro-Wilk test was used to assess normality of distribution. For comparison of groups with continuous variables, parametric unpaired T-test was used for normal distributions and Mann-Whitney test was used for non-normal distributions. Comparison of relative percentage of histological phenotypes were assessed by 2-way ANOVA. Log-rank test was used to compare Kaplan-Meier survival curves. P values throughout the manuscript: *P<0.05, **P<0.01, ***P<0.001, ****P<0.0001, ns: not significant.

PDAC-TMA and TCGA dataset analysis: For the PDAC-TMA dataset, 2-3 cores were selected from FFPE tumor blocks of archived PDAC specimens and TMAs with 1 mm² core area were generated. Serial sections were used for CD4-CD8-Foxp3 and CD11c staining. 129 treatment naïve samples were stained to analyze immune infiltration in these tumors. TCGA survival analyses were performed using Pancreatic adenocarcinoma (PAAD) gene expression data on treatment 172 naïve samples and clinical data downloaded from UCSC Xena (DOI: 10.1038/s41587-020-0546-8). The gene expression was normalized by logarithm 2 in UCSC Xena. The inventors divided the tumor samples into two groups based on the median gene expression and disease-specific survival (DSS) was plotted in prism to generate Kaplan-Meier survival plots.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. F. Balkwill, A. Mantovani, Inflammation and cancer: back to Virchow? Lancet 357, 539-545 (2001).
2. A. B. Lowenfels et al., Pancreatitis and the risk of pancreatic cancer. International Pancreatitis Study Group. N Engl J Med 328, 1433-1437 (1993).
3. D. P. Ryan, T. S. Hong, N. Bardeesy, Pancreatic adenocarcinoma. N Engl J Med 371, 2140-2141 (2014).
4. J. Kirkegard, D. Cronin-Fenton, U. Heide-Jorgensen, F. V. Mortensen, Acute Pancreatitis and Pancreatic Cancer Risk: A Nationwide Matched-Cohort Study in Denmark. Gastroenterology 154, 1729-1736 (2018).
5. V. Corbo, G. Tortora, A. Scarpa, Molecular pathology of pancreatic cancer: from bench-to-bedside translation. Curr Drug Targets 13, 744-752 (2012).
6. G. Kloppel, Chronic pancreatitis, pseudotumors and other tumor-like lesions. Mod Pathol 20 Suppl 1, S113-131 (2007).
7. Cancer Genome Atlas Research Network. Electronic address, N. Cancer Genome Atlas Research, Integrated Genomic Characterization of Pancreatic Ductal Adenocarcinoma. Cancer Cell 32, 185-203 e113 (2017).
8. D. Wei et al., KLF4 Is Essential for Induction of Cellular Identity Change and Acinar-to-Ductal Reprogramming during Early Pancreatic Carcinogenesis. Cancer Cell 29, 324-338 (2016).
9. M. A. Collins et al., Oncogenic Kras is required for both the initiation and maintenance of pancreatic cancer in mice. J Clin Invest 122, 639-653 (2012).
10. A. K. Swidnicka-Siergiejko et al., Chronic inflammation initiates multiple forms of K-Ras-independent mouse pancreatic cancer in the absence of TP53. Oncogene 36, 3149-3158 (2017).
11. R. E. Royal et al., Phase 2 trial of single agent Ipilimumab (anti-CTLA-4) for locally advanced or metastatic pancreatic adenocarcinoma. J Immunother 33, 828-833 (2010).
12. D. T. Le et al., Evaluation of ipilimumab in combination with allogeneic pancreatic tumor cells transfected with a GM-CSF gene in previously treated pancreatic cancer. J Immunother 36, 382-389 (2013).
13. R. Winograd et al., Induction of T-cell Immunity Overcomes Complete Resistance to PD-1 and CTLA-4 Blockade and Improves Survival in Pancreatic Carcinoma. Cancer Immunol Res 3, 399-411 (2015).
14. B. C. Ozdemir et al., Depletion of carcinoma-associated fibroblasts and fibrosis induces immunosuppression and accelerates pancreas cancer with reduced survival. Cancer Cell 25, 719-734 (2014).
15. R. A. Evans et al., Lack of immunoediting in murine pancreatic cancer reversed with neoantigen. JCI Insight 1, (2016).
16. V. P. Balachandran et al., Identification of unique neoantigen qualities in long-term survivors of pancreatic cancer. Nature 551, 512-516 (2017).
17. Y. Zhang et al., Myeloid cells are required for PD-1/PD-L1 checkpoint activation and the establishment of an immunosuppressive environment in pancreatic cancer. Gut 66, 124-136 (2017).
18. Y. Zhang et al., CD4+T lymphocyte ablation prevents pancreatic carcinogenesis in mice. Cancer Immunol Res 2, 423-435 (2014).
19. F. McAllister et al., Oncogenic Kras activates a hematopoietic-to-epithelial IL-17 signaling axis in preinvasive pancreatic neoplasia. Cancer Cell 25, 621-637 (2014).
20. J. E. Jang et al., Crosstalk between Regulatory T Cells and Tumor-Associated Dendritic Cells Negates Anti-tumor Immunity in Pancreatic Cancer. Cell Rep 20, 558-571 (2017).
21. Y. Zhang et al., Regulatory T-cell Depletion Alters the Tumor Microenvironment and Accelerates Pancreatic Carcinogenesis. Cancer Discov 10, 422-439 (2020).
22. A. S. Bedrosian et al., Dendritic cells promote pancreatic viability in mice with acute pancreatitis. Gastroenterology 141, 1915-1926 e1911-1914 (2011).
23. D. G. DeNardo et al., CD4(+) T cells regulate pulmonary metastasis of mammary carcinomas by enhancing protumor properties of macrophages. Cancer Cell 16, 91-102 (2009).
24. M. Guilliams et al., Unsupervised High-Dimensional Analysis Aligns Dendritic Cells across Tissues and Species. Immunity 45, 669-684 (2016).
25. T. T. Chrisikos et al., Molecular regulation of dendritic cell development and function in homeostasis, inflammation, and cancer. Mol Immunol 110, 24-39 (2019).
26. S. Hegde et al., Dendritic Cell Paucity Leads to Dysfunctional Immune Surveillance in Pancreatic Cancer. Cancer Cell 37, 289-307 e289 (2020).

27 K. A. Koelsch, Y. Wang, J. S. Maier-Moore, A. H. Sawalha, J. D. Wren, GFP affects human T cell activation and cytokine production following in vitro stimulation. PLOS One 8, e50068 (2013).
28. R. Stripecke et al., Immune response to green fluorescent protein: implications for gene therapy. Gene Ther 6, 1305-1312 (1999).
29. K. Steinbach, I. Vincenti, D. Merkler, Resident-Memory T Cells in Tissue-Restricted Immune Responses: For Better or Worse? Front Immunol 9, 2827 (2018).
30. Y. Zhou et al., Vaccine efficacy against primary and metastatic cancer with in vitro-generated CD103(+) conventional dendritic cells. J Immunother Cancer 8, (2020).
31. S. R. Hingorani et al., Trp53R172H and KrasG12D cooperate to promote chromosomal instability and widely metastatic pancreatic ductal adenocarcinoma in mice. Cancer Cell 7, 469-483 (2005).
32. A. Rahemtulla et al., Normal development and function of CD8+ cells but markedly decreased helper cell activity in mice lacking CD4. Nature 353, 180-184 (1991).
33. W. P. Fung-Leung et al., CD8 is needed for development of cytotoxic T cells but not helper T cells. Cell 65, 443-449 (1991).
34. S. Kamerkar et al., Exosomes facilitate therapeutic targeting of oncogenic KRAS in pancreatic cancer. Nature 546, 498-503 (2017).
35. H. Ying et al., Oncogenic Kras maintains pancreatic tumors through regulation of anabolic glucose metabolism. Cell 149, 656-670 (2012).
36. C. T. Mayer et al., Selective and efficient generation of functional Batf3-dependent CD103+ dendritic cells from mouse bone marrow. Blood 124, 3081-3091 (2014).
37. J. L. Carstens et al., Spatial computation of intratumoral T cells correlates with survival of patients with pancreatic cancer. Nat Commun 8, 15095 (2017).

What is claimed:

1. A method for treating a subject with pancreatic cancer, the method comprising providing an immunotherapy to the subject, wherein the subject has been determined to have or to have had pancreatitis.

2. The method of claim 1, wherein the pancreatitis is chronic pancreatitis.

3. The method of claim 1, wherein the pancreatitis is acute pancreatitis.

4. The method of any of claims 1-3, wherein the pancreatic cancer is pancreatic ductal adenocarcinoma (PDAC).

5. The method of any of claims 1-4, wherein the immunotherapy is an immune checkpoint blockade therapy.

6. The method of claim 5, wherein the immune checkpoint blockade therapy comprises providing to the subject an antibody or antibody-like molecule capable of binding to an immune checkpoint protein.

7. The method of claim 5, wherein the immune checkpoint blockade therapy comprises providing to the subject a cell comprising a chimeric antigen receptor (CAR) capable of binding to an immune checkpoint protein.

8. The method of claim 6 or 7, wherein the immune checkpoint protein is CTLA-4, PD-1, PDL1, IDO, LAG3, or TIM-3.

9. The method of claim 8, wherein the immune checkpoint protein is PD-1.

10. The method of claim 5, wherein the immune checkpoint blockade therapy comprises two or more immune checkpoint inhibitors.

11. The method of claim 10, wherein the two or more immune checkpoint inhibitors comprise two or more of an anti-PD-1 antibody, an anti PDL1 antibody, and an anti-CTLA4 antibody.

12. The method of any of claims 1-9, wherein pancreatic tissue from the subject was determined to comprise CD11c+ dendritic cells.

13. The method of any of claims 1-12, wherein pancreatic tissue from the subject was determined to comprise tertiary lymphoid structures.

14. The method of any of claims 1-13, further comprising providing to the subject a dendritic cell vaccine.

15. The method of claim 14, wherein the dendritic cell vaccine is an autologous dendritic cell vaccine.

16. The method of claim 14 or 15, wherein the dendritic cell vaccine comprises conventional dendritic cells (cDCs).

17. The method of claim 16, wherein the cDCs are conventional type 1 dendritic cells (cDC1s).

18. The method of any of claims 1-17, wherein the subject was previously treated for pancreatic cancer.

19. The method of claim 18, wherein the subject was previously treated with an immunotherapy.

20. The method of claim 18 or 19, wherein the subject was determined to be resistant to the previous treatment.

21. The method of any of claims 1-20, further comprising providing to the subject an additional cancer therapy.

22. The method of any of claim 21, wherein the additional cancer therapy is chemotherapy, hormone therapy, radiation therapy, surgery, or immunotherapy.

23. A method for treating a subject with pancreatic cancer, the method comprising:
(a) identifying the subject as having pancreatitis or as having previously had pancreatitis; and
(b) providing an immunotherapy to the subject.

24. The method of any of claim 23, wherein the pancreatitis is chronic pancreatitis.

25. The method of any of claim 23, wherein the pancreatitis is acute pancreatitis.

26. The method of any of claims 23-25, wherein (a) comprises testing the subject for one or more symptoms of pancreatitis.

27. The method of any of claims 23-25, wherein (a) comprises detecting an increased level of one or more pancreatic enzymes in the subject relative to a control or healthy subject.

28. The method of claim 27, wherein the one or more pancreatic enzymes comprise amylase or lipase.

29. The method of any of claims 23-28, wherein the pancreatic cancer is pancreatic ductal adenocarcinoma (PDAC).

30. The method of any of claims 23-29 wherein the immunotherapy is an immune checkpoint blockade therapy.

31. The method of claim 30, wherein the immune checkpoint blockade therapy comprises providing to the subject an antibody or antibody-like molecule capable of binding to an immune checkpoint protein.

32. The method of claim 30, wherein the immune checkpoint blockade therapy comprises providing to the subject a cell comprising a chimeric antigen receptor (CAR) capable of binding to an immune checkpoint protein.

33. The method of claim 31 or 32, wherein the immune checkpoint protein is CTLA-4, PD-1, PDL1, IDO, LAG3, or TIM-3.

34. The method of claim 33, wherein the immune checkpoint protein is PD-1.

35. The method of claim 30, wherein the immune checkpoint blockade therapy comprises two or more immune checkpoint inhibitors.

36. The method of claim 35, wherein the two or more immune checkpoint inhibitors comprise two or more of an anti-PD-1 antibody, an anti PDL1 antibody, and an anti-CTLA4 antibody.

37. The method of any of claims 23-36, further comprising detecting CD11c+ dendritic cells in pancreatic tissue from the subject.

38. The method of any of claims 23-37, further comprising detecting tertiary lymphoid structures in pancreatic tissue from the subject.

39. The method of any of claims 23-38, further comprising providing to the subject a dendritic cell vaccine.

40. The method of claim 39, wherein the dendritic cell vaccine is an autologous dendritic cell vaccine.

41. The method of claim 39 or 40, wherein the dendritic cell vaccine comprises conventional dendritic cells (cDCs).

42. The method of claim 41, wherein the cDCs are conventional type 1 dendritic cells (cDC1s).

43. The method of any of claims 23-40, wherein the subject was previously treated for pancreatic cancer.

44. The method of claim 43, wherein the subject was previously treated with an immunotherapy.

45. The method of claim 43 or 44, wherein the subject was determined to be resistant to the previous treatment.

46. The method of any of claims 23-45, further comprising providing to the subject an additional cancer therapy.

47. The method of claim 46. wherein the additional cancer therapy is chemotherapy, radiation therapy, hormone therapy, surgery, or immunotherapy.

48. A method for treating a subject with pancreatic cancer, the method comprising determining whether the subject has or has previously had pancreatitis and:
(a) providing an immunotherapy to the subject if the subject is determined to have or to have previously had pancreatitis; or
(b) providing an alternative cancer therapy to the subject if the subject is determined to have never had pancreatitis, wherein the alternative cancer therapy does not comprise an immunotherapy.

49. The method of claim 48, wherein the alternative cancer therapy is chemotherapy, hormone therapy, radiation therapy, or surgery.

50. The method of claim 48 or 49, wherein the pancreatitis is chronic pancreatitis.

51. The method of claim 48 or 49, wherein the pancreatitis is acute pancreatitis.

52. The method of any of claims 48-51, wherein determining whether the subject has or has previously had pancreatitis comprises testing the subject for one or more symptoms of pancreatitis.

53. The method of any of claims 48-51, wherein determining whether the subject has or has previously had pancreatitis comprises detecting an increased level of one or more pancreatic enzymes in the subject relative to a control or healthy subject.

54. The method of claim 53, wherein the one or more pancreatic enzymes comprise amylase or lipase.

55. The method of any of claims 48-54, wherein the pancreatic cancer is pancreatic ductal adenocarcinoma (PDAC).

56. The method of any of claims 48-55, wherein the immunotherapy is an immune checkpoint blockade therapy.

57. The method of claim 56, wherein the immune checkpoint blockade therapy comprises providing to the subject an antibody or antibody-like molecule capable of binding to an immune checkpoint protein.

58. The method of claim 56, wherein the immune checkpoint blockade therapy comprises providing to the subject a cell comprising a chimeric antigen receptor (CAR) capable of binding to an immune checkpoint protein.

59. The method of claim 57 or 58, wherein the immune checkpoint protein is CTLA-4, PD-1, PDL1, IDO, LAG3, or TIM-3.

60. The method of claim 59, wherein the immune checkpoint protein is PD-1.

61. The method of claim 56, wherein the immune checkpoint blockade therapy comprises two or more immune checkpoint inhibitors.

62. The method of claim 61, wherein the two or more immune checkpoint inhibitors comprise two or more of an anti-PD-1 antibody, an anti PDL1 antibody, and an anti-CTLA4 antibody.

63. The method of any of claims 48-62, further comprising detecting CD11c+ dendritic cells in pancreatic tissue from the subject.

64. The method of any of claims 48-63, further comprising detecting tertiary lymphoid structures in pancreatic tissue from the subject.

65. The method of any of claims 48-64, further comprising providing to the subject a dendritic cell vaccine.

66. The method of claim 65, wherein the dendritic cell vaccine is an autologous dendritic cell vaccine.

67. The method of claim 65 or 66, wherein the dendritic cell vaccine comprises conventional dendritic cells (cDCs).

68. The method of claim 67, wherein the cDCs are conventional type 1 dendritic cells (cDC1s).

69. The method of any of claims 48-66, wherein the subject was previously treated for pancreatic cancer.

70. The method of any of claims 48-69, wherein the subject was previously treated with an immunotherapy.

71. The method of any of claim 70, wherein the subject was determined to be resistant to the previous treatment.

72. A method for treating a subject with pancreatic cancer, the method comprising:
(a) inducing pancreatitis in the subject; and
(b) subsequent to (a), providing to the subject an immunotherapy.

73. The method of claim 72, wherein (a) comprises providing an infectious agent to the subject.

74. The method of claim 72 or 73, wherein (a) comprises pancreatic surgery.

75. The method of any of claims 72-74, wherein the pancreatitis is chronic pancreatitis.

76. The method of any of claims 72-74, wherein the pancreatitis is acute pancreatitis.

77. The method of any of claims 72-76, wherein the pancreatic cancer is pancreatic ductal adenocarcinoma (PDAC).

78. The method of any of claims 72-77, wherein the immunotherapy is an immune checkpoint blockade therapy.

79. The method of claim 78, wherein the immune checkpoint blockade therapy comprises providing to the subject an antibody or antibody-like molecule capable of binding to an immune checkpoint protein.

80. The method of claim 78, wherein the immune checkpoint blockade therapy comprises providing to the subject a cell comprising a chimeric antigen receptor (CAR) capable of binding to an immune checkpoint protein.

81. The method of claim 79 or 80, wherein the immune checkpoint protein is CTLA-4, PD-1, IDO, LAG3, or TIM-3.

82. The method of claim 81, wherein the immune checkpoint protein is PD-1.

83. The method of any of claims 72-82, further comprising providing to the subject a dendritic cell vaccine.

84. The method of claim 83, wherein the dendritic cell vaccine is an autologous dendritic cell vaccine.

85. The method of any of claims 72-84, wherein the subject was previously treated for pancreatic cancer.

86. The method of claim 85, wherein the subject was previously treated with an immunotherapy.

87. The method of claim 85 or 86, wherein the subject was determined to be resistant to the previous treatment.

88. The method of any of claims 72-87, further comprising providing to the subject an additional cancer therapy.

89. The method of claim 88, wherein the additional cancer therapy is chemotherapy, radiation therapy, hormone therapy, surgery, or immunotherapy.

90. A method for treating a subject with pancreatic cancer, the method comprising administering an effective amount of a dendritic cell vaccine and an immunotherapy to the subject.

91. The method of claim 90, wherein the dendritic cell vaccine and the immunotherapy are administered substantially simultaneously.

92. The method of claim 90, wherein the dendritic cell vaccine and the immunotherapy are administered sequentially.

93. The method of claim 92, wherein the dendritic cell vaccine is administered prior to the immunotherapy.

94. The method of claim 92, wherein the immunotherapy is administered prior to the dendritic cell vaccine.

95. The method of any of claims 90-94, wherein the pancreatic cancer is pancreatic ductal adenocarcinoma (PDAC).

96. The method of any of claims 90-95, wherein the immunotherapy is an immune checkpoint blockade therapy.

97. The method of claim 96, wherein the immune checkpoint blockade therapy comprises providing to the subject an antibody or antibody-like molecule capable of binding to an immune checkpoint protein.

98. The method of claim 96, wherein the immune checkpoint blockade therapy comprises providing to the subject a cell comprising a chimeric antigen receptor (CAR) capable of binding to an immune checkpoint protein.

99. The method of claim 97 or 98, wherein the immune checkpoint protein is CTLA-4, PD-1, PDL1, IDO, LAG3, or TIM-3.

100. The method of claim 99, wherein the immune checkpoint protein is PD-1.

101. The method of claim 96, wherein the immune checkpoint blockade therapy comprises two or more immune checkpoint inhibitors.

102. The method of claim 101, wherein the two or more immune checkpoint inhibitors comprise two or more of an anti-PD-1 antibody, an anti PDL1 antibody, and an anti-CTLA4 antibody.

103. The method of any of claims 90-102, wherein the dendritic cell vaccine is an autologous dendritic cell vaccine.

104. The method of any of claims 90-103, wherein the dendritic cell vaccine comprises conventional dendritic cells (cDCs).

105. The method of claim 104, wherein the cDCs are conventional type 1 dendritic cells (cDC1s).

106. The method of any of claims 90-105, wherein the subject was previously treated for pancreatic cancer.

107. The method of claim 106, wherein the subject was previously treated with an immunotherapy.

108. The method of claim 106 or 107, wherein the subject was determined to be resistant to the previous treatment.

109. The method of any of claims 90-108, further comprising providing to the subject an additional cancer therapy.

110. The method of any of claim 109, wherein the additional cancer therapy is chemotherapy, hormone therapy, radiation therapy, surgery, or immunotherapy.

111. A method for treating a subject for pancreatic cancer, the method comprising administering an immunotherapy to a subject determined to have tertiary lymphoid structures in pancreatic cancer tissue from the subject.

112. The method of claim 111, wherein the immunotherapy is an immune checkpoint blockade therapy.

113. A method for treating a subject for pancreatic cancer, the method comprising:
(a) detecting tertiary lymphoid structures in pancreatic tissue of the subject; and
(b) administering an immunotherapy to the subject.

114. A method for treating a subject for pancreatic cancer, the method comprising:
(a) inducing formation of tertiary lymphoid structures in pancreatic tissue of the subject; and
(b) administering an immunotherapy to the subject.

115. A method for identifying subjects with pancreatic cancer as candidates for immune checkpoint blockade therapy, the method comprising:
(a) determining whether each subject of a group of subjects with pancreatic cancer has or has previously had pancreatitis; and
(b) identifying subjects from the group of subjects that have or have previously had pancreatitis as candidates for immune checkpoint blockade therapy.
